(19) United States
(12) Patent Application Publication
(10) Pub. No.: US 2010/0098788 A1
(43) Pub. Date: Apr. 22, 2010

(54) Extracts of Curcuma and Methods of Use Thereof

(76) Inventors: Randall S. Alberte, Estero, FL (US); William P. Roschek, JR., Naples, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/580,778

(22) Filed: Oct. 16, 2009

Related U.S. Application Data
(60) Provisional application No. 61/105,995, filed on Oct. 16, 2008.

(51) Int. Cl. A61K 36/906; A61P 25/28; A61K 31/12

(52) U.S. Cl. 424/756; 514/683

(57) ABSTRACT

The present invention relates in part to turmeric extracts that are useful for treating or preventing neurodegenerative disorders. Another aspect of the invention relates in part to turmeric extracts that are useful for treating or preventing inflammatory disorders. In some embodiments, the extracts comprise at least one compound selected from the group consisting of 25 to 500 μg bamosamine, 25 to 750 μg echinaxanthol, 100 to 3,000 μg bisdemethoxycurcumin, 50 to 500 μg daphniyunnine E and 500 to 75,000 μg curcumin per 100 mg of extract. Another aspect of the invention relates to pharmaceutical compositions comprising the aforementioned extracts. Another aspect of the invention relates to methods of treating or preventing neurodegenerative disorders comprising administering to a subject in need thereof an effective amount of the aforementioned extracts or compositions. Another aspect of the invention relates to methods of making the aforementioned extracts.

### RELATED APPLICATIONS

This application claims the benefit of priority to U.S. Provisional Application Ser. No. 61/105,995, filed on Oct. 16, 2008, which is herein incorporated by reference in its entirety.

### BACKGROUND OF THE INVENTION

Alzheimer's disease (AD), the most common cause of dementia among the elderly, is characterized by cognitive deterioration, progressive memory loss, and behavioral problems. Pathologically, AD is characterized post mortem by the presence of senile plaques, neurofibrillary tangles, and neuronal cell loss. The accumulation of β-amyloid (Aβ), produced as a cleavage product of the amyloid precursor protein (APP), both as soluble aggregate oligomers and senile plaques is a neuropathological hallmark of AD (D. J. Selkoe, 1994. Alzheimer's disease: a central role for amyloid, J. Neuropathol. Exp. Neurol. 53:438-447). A fundamental aspect of the current Aβ cascade hypothesis is that Aβ accumulation in the brain initiates a series of pathological reactions that result in tau aggregation and neuronal dysfunction that are the primary causes of dementia (T. E. Golde, D. Dickson and M. Hutton, 2006. Filling the gaps in the Aβ hypothesis of Alzheimer's disease, Curr. Alzheimer Res. 3 :421-430)

Roles for neuroinflammation and oxidative damage have also been implicated in neurodegeneration, and may play an important role in the neuropathogenesis of AD (Y. Christen, 2000. Oxidative stress and Alzheimer disease, Am J Clin Nutr. 71:621S-629S; G. M. Cole, T. Morihara, G. P. Lim, F. Yang, A. Begum and S. A. Frautschy, 2004. NSAID and antioxidant prevention of Alzheimer's disease: lessons from in vitro and animal models, Ann NY Acad Sci. 1035:68-84). For example, Aβ can produce H₂O₂ (X. Huang, C. S. Atwood, M. A. Hartshorn, G. Multhaup, L. E. Goldstein, R. C. Scarpa, M. P. Cuajungco, D. N. Gray, J. Lim, R. D. Moir, R. E. Tanzi and A. I. Bush, 1999. The A beta peptide of Alzheimer's disease directly produces hydrogen peroxide through metal ion reduction, Biochemistry. 38:7609-7616) and reactive oxygen species (ROS) that may mediate plaque-induced neurotoxicity (J. El Khoury, S. E. Hickman, C. A. Thomas, J. D. Loike and S. C. Silverstein, 1998. Microglia, scavenger receptors, and the pathogenesis of Alzheimer's disease, Neurobiol Aging. 19:581-84; M. E. McLellan, S. T. Kajdasz, B. T. Hyman and B. J. Bacskai, 2003. In vivo imaging of reactive oxygen species specifically associated with thioflavine S-positive amyloid plaques by multiphoton microscopy, J Neurosci. 23:2212-2217; M. Garcia-Alloza, E. M. Robbins, S. X. Zhang-Nunes, S. M. Purcell, R. A. Betensky, S. Raju, C. Prada, S. M. Greenberg, B. J. Bacskai and M. P. Frosch, 2006. Characterization of amyloid deposition in the APPswe/PS1dE9 mouse model of Alzheimer disease, Neurobiol Dis. 24:516-524). Recently it has been shown that inhibition of mitochondrial respiratory capacity and oxidative stress elevates β-secretase protein levels and activity as well as Aβ levels (K. Xiong, H. Cai, X.-G. Luo, R. G. Struble, R. W. Clough and X.-X. Yan, 2007. Mitochondrial respiratory inhibition and oxidative stress elevate β-secretase (BACE1) proteins and activity in vivo in the rat retina, Exp Brain Res. 181:435-446). In addition, mechanical disruption of mitochondrial electron transport activities by amyloid accumulation in this organelle leads to loss of ROS scavenging function as well as loss in energetic capabilities required for neuronal cell maintenance and activities (V. Chauhan and A. Chauhan, 2006. Oxidative stress in Alzheimer's disease, Pathophysiology. 13:195-208; F. M. LaFerla, K. N. Green and S. Oddo, 2007. Intracellular amyloid in Alzheimer's disease, Nat Rev Neurosci. 8:449-509).

At present, the number of therapeutic options for AD is severely limited (R. E. Becker and N. H. Greig, 2008. Alzheimer's disease drug development in 2008 and beyond: problems and opportunities, Curr. Alzheimer Res. 5:346-357). Currently marketed drugs for AD do not prevent or reverse this disease and are approved only for the management of symptoms (M. N. Pangalos, L. E. Schechter and O. Hurko, 2007. Drug development for CNS disorders: strategies for balancing risk and reducing attrition, Nat Rev Drug Discov. 6:521-532). Driven by the clear unmet medical need and a better understanding of the biology and pathophysiology of AD, the number of drugs in development for this indication has increased dramatically in recent years (I. Melnikova, 2007. Therapies for Alzheimer's disease, Nat Rev Drug Discov. 6:341-342). Because drug discovery using synthetic drugs is expensive, complex, and vastly inefficient, many groups have turned their attention to screen natural products and botanical extracts, especially where therapeutic uses and benefits have been documented by traditional medicine systems (D. S. Fabricant and N. R. Farnsworth, 2001. The value of plants used in traditional medicine for drug discovery, Environ Health Perspect. 109 Suppl 1:69-75; B. Patwardhan, D. Warude, P. Pushpangadan and N. Bhatt, 2005. Ayurveda and traditional Chinese medicine: a comparative overview, Evid Based Complement Alternat Med. 2:465-473). For example, it was recently found that EGCG, the major polyphenolic found in green tea, works both in vitro and in vivo to reduce amyloid production by promoting α-secretase activity (K. Rezai-Zadeh, D. Shytle, N. Sun, T. Mori, H. Hou, D. Jeanniton, J. Ehrhart, K. Townsend, J. Zeng, D. Morgan, J. Hardy, T. Town and J. Tan, 2005. Green tea epigallocatechin-3-gallate (EGCG) modulates amyloid precursor protein cleavage and reduces cerebral amyloidosis in Alzheimer transgenic mice, J Neurosci. 25:8807-8814). In addition, curcumin represents a hopeful approach for treating, delaying, and/or preventing the progression of AD (G. M. Cole, T. Morihara, G. P. Lim, F. Yang, A. Begum and S. A. Frautschy, 2004. NSAID and antioxidant prevention of Alzheimer's disease: lessons from in vitro and animal models, Ann NY Acad Sci. 1035:68-84).

Traditionally known for its an anti-inflammatory effects, curcumin has been shown, in the last two decades, to be a potent therapeutic agent with reported beneficial effects in arthritis, allergy, asthma, atherosclerosis, heart disease, diabetes, and cancer (S. Ray, N. Chattopadhyay, A. Mitra, M. Siddiqi and A. Chatterjee, 2003. Curcumin exhibits antimetastatic properties by modulating integrin receptors, collagenase activity, and expression of Nm23 and E-cadherin, J Environ Pathol Toxicol Oncol. 22:49-58; G. M. Cole, B. Teter and S. A. Frautschy, 2007. Neuroprotective effects of curcumin, Adv Exp Med Biol. 595:197-212; S. S. Bhandarkar and J. L. Arbiser, 2007. Curcumin as an inhibitor of angiogenesis, Adv Exp Med Biol. 595:185-195; G. Kuttan, K. B. Kumar, C. Guruvayoorappan and R. Kuttan, 2007. Antitumor, anti-invasion, and antimetastatic effects of curcumin, Adv Exp Med Biol. 595:173-184; V. P. Menon and A. R. Sudheer, 2007. Antioxidant and anti-inflammatory properties of curcumin, Adv Exp Med Biol. 595:105-125). In vitro studies have shown that curcumin attenuates inflammatory activation of brain microglial cells (H. Y. Kim, E. J. Park, E. H. Joe and I. Jou, 2003. Curcumin suppresses Janus kinase-STAT inflammatory signaling through activation of Src homology 2 domain-containing tyrosine phosphatase 2 in brain microglia, J Immunol. 171:6072-6079; K. K. Jung, H. S. Lee, J. Y. Cho, W. C. Shin, M. H. Rhee, T. G. Kim, J. H. Kang, S. H. Kim, S. Hong and S. Y. Kang, 2006 Inhibitory effect of curcumin on nitric oxide production from lipopolysaccharide-activated primary microglia, Life Sci. 79:2022-2031). Curcumin also inhibits the formation of Aβ oligomers and fibrils in vitro (K. Ono, K. Hasegawa, H. Naiki and M. Yamada, 2004. Curcumin has potent anti-amyloidogenic effects for Alzheimer's beta-amyloid fibrils in vitro, J Neurosci Res. 75:742-750; F. Yang, G. P. Lim, A. N. Begum, O. J. Ubeda, M. R. Simmons, S. S. Ambegaokar, P. P. Chen, R. Kayed, C. G. Glabe, S. A. Frautschy and G. M. Cole, 2005. Curcumin inhibits formation of amyloid beta oligomers and fibrils, binds plaques, and reduces amyloid in vivo, J Biol Chem. 280:5892-5901). Other studies have shown that curcumin prevents neuronal damage (P. K. Shukla, V. K. Khanna, M. Y. Khan and R. C. Srimal, 2003. Protective effect of curcumin against lead neurotoxicity in rat, Hum Exp Toxicol. 22:653-658), reduces both oxidative damage and amyloid accumulation in a transgenic mouse model of AD (G. P. Lim, T. Chu, F. Yang, W. Beech, S. A. Frautschy and G. M. Cole, 2001. The curry spice curcumin reduces oxidative damage and amyloid pathology in an Alzheimer transgenic mouse, J. Neurosci. 21:8370-8377; S. A. Frautschy, W. Hu, P. Kim, S. A. Miller, T. Chu, M. E. Harris-White and G. M. Cole, 2001. Phenolic anti-inflammatory antioxidant reversal of Abeta-induced cognitive deficits and neuropathology, Neurobiol. Aging. 22:993-1005; S. K. Sandur, H. Ichikawa, M. K. Pandey, A. B. Kunnumakkara, B. Sung, G. Sethi and B. B. Aggarwal, 2007. Role of pro-oxidants and antioxidants in the anti-inflammatory and apoptotic effects of curcumin (diferuloylmethane), Free Radic. Biol. Med. 43:568-580; F. Yang, G. P. Lim, A. N. Begum, O. J. Ubeda, M. R. Simmons, S. S. Ambegaokar, P. P. Chen, R. Kayed, C. G. Glabe, S. A. Frautschy and G. M. Cole, 2005. Curcumin inhibits formation of amyloid beta oligomers and fibrils, binds plaques, and reduces amyloid in vivo, J. Biol. Chem. 280:5892-5901). Curcumin has been shown to be active in amyloid aggregation and secretion in animal models (F. Yang, G. P. Lim, A. N. Begum, O. J. Ubeda, M. R. Simmons, S. S. Ambegaokar, P. P. Chen, R. Kayed, C. G. Glabe, S. A. Frautschy and G. M. Cole, 2005. Curcumin inhibits formation of amyloid beta oligomers and fibrils, binds plaques, and reduces amyloid in vivo, J Biol Chem. 280:5892-5901; P. K. Shukla, V. K. Khanna, M. Y. Khan and R. C. Srimal, 2003. Protective effect of curcumin against lead neurotoxicity in rat, Hum Exp Toxicol. 22:653-658; K. Ono, K. Hasegawa, H. Naiki and M. Yamada, 2004. Curcumin has potent anti-amylodogenic effects for Alzheimer's beta-amyloid fibrils in vitro, J. Neurosci Res. 75:742-750). Its activity is often ascribed to its role in ROS scavenging and reduction in neurotoxicity.

Recently, Garcia et al. (M. Garcia-Alloza, L. A. Borrelli, A. Rozkalne, B. T. Hyman and B. J. Bacskai, 2007. Curcumin labels amyloid pathology in vivo, disrupts existing plaques, and partially restores distorted neurites in an Alzheimer mouse model, J Neurochem.) used multiphoton microscopy (MPM) and longitudinal imaging to evaluate in vivo and in real-time the effects of systemic curcumin administration on existing Aβ deposits using aged APPswe/PS1dE9 transgenic mice. They found that curcumin clears and reduces plaques and partially restores the altered neuronal pathology near and away from plaques (M. Garcia-Alloza, L. A. Borrelli, A. Rozkalne, B. T. Hyman and B. J. Bacskai, 2007. Curcumin labels amyloid pathology in vivo, disrupts existing plaques, and partially restores distorted neurites in an Alzheimer mouse model, J. Neurochem. 102:1095-1104). This study further supports evidence that curcumin has beneficial effects in reducing the pathology and neurotoxicity of AD in transgenic mice. Lastly, human clinical trials have shown that curcumin is safe and has broad anti-inflammatory properties (P. R. Holt, S. Katz and R. Kirshoff, 2005. Curcumin therapy in inflammatory bowel disease: a pilot study, Dig Dis Sci. 50:2191-2193).

While investigations have shown anti-amyloidogenic effects of curcumin, no research to date has examined “optimized” turmeric extracts enriched in the curcuminoids. Commercially available curcumin extracts used for research and for clinical trials vary considerably, but often contain about 75% curcumin (Cur), 15% demethoxycurcumin (DMC), and 5% bisdemethoxycurcumin (BDMC). In addition, some extracts also contain very low levels of tetrahydrocurcumin (THC), one of the naturally occurring metabolites of curcumin. Various studies have shown that curcumin and DMC are less stable than BDMC, whereas the reduced curcumin metabolite, THC, is the most stable curcuminoid. Turmeric and most commercial turmeric extracts are also rich in the lipid-soluble turmerones. The turmerones include several species with ar-turmerone, α-turmerone and β-turmerone typically being the most abundant in turmeric. The precise role of turmerones in AD is unclear, though they have established anti-inflammatory and anti-oxidative activities which could reduce neurotoxicity (S. Jain, S. Shrivastava, S. Nayak and S. Sumbhate, 2007. PHCOG MAG: Plant Review. Recent trends in Curcuma longa Linn., Pharmacog. Revs. 1:119-128).

There are three secretases (proteases) that process APP (E. H. Koo, S. L. Squazzo, D. J. Selkoe and C. H. Koo, 1996. Trafficking of cell-surface amyloid beta-protein precursor. I. Secretion, endocytosis and recycling as detected by labeled monoclonal antibody, J. Cell Sci. 109 (Pt 5):991-998; K. S. Vetrivel and G. Thinakaran, 2006. Amyloidogenic processing of beta-amyloid precursor protein in intracellular compartments, Neurology. 66:S69-S73). These include α-, β- and γ-secretases which are localized on the endoplasmic reticulum (ER) membrane. The secretase enzymes involved in processing of APP to Aβ are current therapeutic targets for AD treatment (G. Shoba, D. Joy, T. Joseph, M. Majeed, R. Rajendran and P. S. Srinivas, 1998. Influence of piperine on the pharmacokinetics of curcumin in animals and human volunteers, Planta Med. 64:353-356) Inhibitors of γ-secretase have been shown to reduce significantly levels of β-amyloid level in brain pointing to the key role of inhibition of amyloid secretion to disease treatment (H. F. Dovey, V. John, J. P. Anderson, L. Z. Chen, P. de Saint Andrieu, L. Y. Fang, S. B. Freedman, B. Folmer, E. Goldbach, E. J. Holsztynska, K. L. Hu, K. L. Johnson-Wood, S. L. Kennedy, D. Kholodenko, J. E. Knops, L. H. Latimer, M. Lee, Z. Liao, I. M. Lieberburg, R. N. Motter, L. C. Mutter, J. Nietz, K. P. Quinn, K. L. Sacchi, P. A. Seubert, G. M. Shopp, E. D. Thorsett, J. S. Tung, J. Wu, S. Yang, C. T. Yin, D. B. Schenk, P. C. May, L. D. Altstiel, M. H. Bender, L. N. Boggs, T. C. Britton, J. C. Clemens, D. L. Czilli, D. K. Dieckman-McGinty, J. J. Droste, K. S. Fuson, B. D. Gitter, P. A. Hyslop, E. M. Johnstone, W. Y. Li, S. P. Little, T. E. Mabry, F. D. Miller and J. E. Audia, 2001. Functional gamma-secretase inhibitors reduce beta-amyloid peptide levels in brain, J. Neurochem. 76:173-181; S. B. Roberts, 2002. Gamma-secretase inhibitors and Alzheimer's disease, Adv. Drug Del. Rev. 54:1579-1588; S. L. Cole and R. Vassar, 2008. BACE1 structure and function in health and Alzheimer's disease, Curr. Alzheimer Res. 5:100-120; A. K. Ghosh, N. Kumaragurubaran, L. Hong, G. Koelsh and J. Tang, 2008. Memapsin 2 (beta-secretase) inhibitors: drug development, Curr. Alzheimer Res. 5:121-131). It is unlikely that any of the known curcuminoids are significant inhibitors of these proteases, though the identification of amyloid secretase inhibitors is clearly a high priority therapeutic target for Alzheimer's disease. It has been shown that the brain protein FE65 binds to and increases secretion of β-amyloids, and that inhibition of the binding could be an important therapeutic target as well (S. L. Sabo, L. M. Lanier, A. F. Ikin, O. Khorkova, S. Sahasrabudhe, P. Greengard and J. D. Buxbaum, 1999. Regulation of beta-amyloid secretion by FE65, an amyloid protein precursor-binding protein, J. Biol. Chem. 274:7952-7957).

The hallmark of Alzheimer's disease is the appearance of twisted fibrils in brain tissue as described in 1906 when the disease was first defined. The fibrils are made up of amyloids and tau proteins. Tau proteins interact with tubulin to stabilize microtubules and promote tubulin assembly into microtubules. Tau has two ways of controlling microtubule stability: isoforms and phosphorylation. Six tau isoforms exist in brain tissue, and they are distinguished by their number of binding domains. Phosphorylation of tau is regulated by a host of kinases. For example, PKN, a serine/threonine kinase. When PKN is activated, it phosphorylates tau, resulting in disruption of microtubule organization (T. Taniguchi, T. Kawamata, H. Mukai, H. Hasegawa, T. Isagawa, M. Yasuda, T. Hashimoto, A. Terashima, M. Nakai, H. Mori, Y. Ono and C. Tanaka, 2001. Phosphorylation of tau is regulated by PKN, J. Biol. Chem. 276:10025-10031). Hyperphosphorylation of the tau protein (tau inclusions), however, can result in the self-assembly of tangles of paired helical filaments and straight filaments, which are involved in the pathogenesis of Alzheimer's disease and other tauopathologies (A. Alonso, T. Zaidi, M. Novak, I. Grundke-Iqbal and K. Iqbal, 2001. Hyperphosphorylation induces self-assembly of tau into tangles of paired helical filaments/straight filaments, Proc. Natl. Acad. Sci. USA. 98:6923-6928). Tau protein is a highly soluble microtubule-associated protein (MAP). The tau gene locates on chromosome 17q21, containing 16 exons. Thus, in the human brain, the tau proteins constitute a family of six isoforms with the range from 352-441 amino acids. All of the six tau isoforms are present in an often hyperphosphorylated state in paired helical filaments in Alzheimer's disease brain. When misfolded, this otherwise very soluble protein can form extremely insoluble aggregates that contribute to a number of neurodegenerative diseases (M. Morishima-Kawashima, M. Hasegawa, K. Takio, M. Suzuki, H. Yoshida, A. Watanabe, K. Titani and Y. Ihara, 1995. Hyperphosphorylation of tau in PHF, Neurobiol. Aging. 16:365-371; discussion 371-380).

One important question, in this regard, is how the various chemical species contained in an enriched turmeric extract affects the bioavailability and bioactivity of curcumin and/or other active compounds present. The known curcuminoids possess low bioavailability (G. Shoba, D. Joy, T. Joseph, M. Majeed, R. Rajendran and P. S. Srinivas, 1998. Influence of piperine on the pharmacokinetics of curcumin in animals and human volunteers, Planta Med. 64:353-356) therefore key to the in vivo activity of turmeric extracts are bioavailable forms of the bioactives. Also key for CNS active extracts or compounds, is their ability to cross the blood brain barrier (L. K. Wing, H. A. Behanna, L. J. Van Eldik, D. M. Watterson and H. Ralay Ranaivo, 2006. De novo and molecular target-independent discovery of orally bioavailable lead compounds for neurological disorders, Curr. Alzheimer Res. 3:205-214). There is an increasing need for new therapeutics and alternative treatments to address β-amyloid aggregation and secretion as means to treat or prevent Alzheimer's disease, as to date there are no effective treatments for this progressive and debilitating disease.

Optimized botanical extracts must be developed to produce standardized, dose-reliable, and concentrated botanical extracts necessary to not only meet FDA regulations for botanical drug development, but to provide efficacious and safe herbal medicines. Moreover, optimized botanical extracts under IND for human therapeutic indications must be produced in facilities following GMP and cGMP standards. The development of Direct Analysis in Real Time (DART) Time-of Flight Mass spectrometry (R. B. Cody, J. A. Laramee and H. D. Durst, 2005. Versatile new ion source for the analysis of materials in open air under ambient conditions, Anal Chem. 77:2297-2302) has allowed for the rapid characterization of the chemical complexity of botanical extracts, and has allowed for the chemical compositions of standardized extracts to be defined.

### SUMMARY OF THE INVENTION

One aspect of the invention relates to a turmeric extract comprising at least one compound selected from the group consisting of 25 to 500 μg bamosamine, 25 to 750 μg echinaxanthol, 100 to 3,000 μg bisdemethoxycurcumin, 50 to 500 μg daphniyunnine E and 500 to 75,000 μg curcumin per 100 mg of extract.

In another embodiment, the extract further comprises at least one compound selected from the group consisting of 50 to 500 μg decadienal/santolina epoxide, 10 to 500 μg eugenol, 0200 to 3,000 μg methoxycoumarin, 100 to 2,000 μg elijopyrone D, 100 to 5,000 μg vitamin H (biotin), and 50 to 500 μg epierythrostominol per 100 mg of extract.

In another embodiment, the turmeric extract further comprises at least one compound selected from the group consisting 50 to 1,000 μg lysine, 100 to 3,000 μg methoxycoumarin, 10 to 500 μg ethoxycoumarin, 10 to 500 μg α-phenylindol, 50 to 1,000 μg 3,4-dihydroscopoletin, 50 to 5,000 μg vasicinone, 50 to 5,000 μg 11-epileontidane, 10 to 500 μg methoxyflavanone, 50 to 500 μg aconitic acid triethyl ester, 50 to 500 μg 5,7-dimethoxyflavanone, 10 to 1,000 μg piperine, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

In another embodiment, the turmeric extract comprises 25 to 500 μg bamosamine, 25 to 750 μg echinaxanthol, 100 to 3,000 μg bisdemethoxycurcumin, 50 to 500 μg daphniyunnine E, 500 to 75,000 μg curcumin, 50 to 500 μg decadienal/santolina epoxide, 10 to 500 μg eugenol, 0200 to 3,000 μg methoxycoumarin, 100 to 2,000 μg elijopyrone D, and 50 to 500 μg epierythrostominol per 100 mg of extract.

In another embodiment, the turmeric extract comprises 25 to 500 μg bamosamine, 25 to 750 μg echinaxanthol, 100 to 3,000 μg bisdemethoxycurcumin, 50 to 500 μg daphniyunnine E, 500 to 75,000 μg curcumin, 50 to 1,000 μg lysine, 100 to 3,000 μg methoxycoumarin, 10 to 500 μg ethoxycoumarin, 10 to 500 μg α-phenylindol, 50 to 1,000 μg 3,4-dihydroscopoletin, 50 to 5,000 μg vasicinone, 50 to 5,000 μg 11-epileontidane, 10 to μg methoxyflavanone, 50 to 500 μg aconitic acid triethyl ester, 50 to 500 μg 5,7-dimethoxyflavanone, 10 to 1,000 μg piperine, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

In another embodiment, the turmeric extract comprises 25 to 500 μg bamosamine, 25 to 750 μg echinaxanthol, 100 to 3,000 μg bisdemethoxycurcumin, 50 to 500 μg daphniyunnine E, 500 to 75,000 μg curcumin, 50 to 500 μg decadienal/santolina epoxide, 10 to 500 μg eugenol, 0200 to 3,000 μg methoxycoumarin, 100 to 2,000 μg elijopyrone D, 50 to 500 μg epierythrostominol, 50 to 1,000 pt.g lysine, 10 to 500 μg ethoxycoumarin, 10 to 500 μg α-phenylindol, 50 to 1,000 μg 3,4-dihydroscopoletin, 50 to 5,000 μg vasicinone, 50 to 5,000 μg 11-epileontidane, 10 to 500 μg methoxyflavanone, 50 to 500 μg aconitic acid triethyl ester, 50 to 500 μg 5,7-dimethoxyflavanone, 10 to 1,000 μg piperine, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin.

In another embodiment, the turmeric extract comprises 25 to 750 μg echinaxanthol, 100 to 3,000 μg bisdemethoxycurcumin, 500 to 75,000 μg curcumin per 100 mg of extract, 50 to 500 μg decadienal/santolina epoxide, 10 to 500 μg eugenol, and 100 to 5,000 μg vitamin H (biotin) per 100 mg of extract.

In another embodiment, the turmeric extract comprises 25 to 750 μg echinaxanthol, 100 to 3,000 μg bisdemethoxycurcumin, 500 to 75,000 μg curcumin per 100 mg of extract, 100 to 1,000 μg ephemeranthone, 1000 to 10,000 μg demethoxycurcumin, 100 to 1,000 μg zopfinol, and 100 to 1,000 μg (+)-fargesin per 100 mg of extract.

In another embodiment, the turmeric extract comprises 25 to 750 μg echinaxanthol, 100 to 3,000 μg bisdemethoxycurcumin, 500 to 75,000 μg curcumin per 100 mg of extract, 50 to 500 μg decadienal/santolina epoxide, 10 to 500 μg eugenol, 100 to 5,000 μg vitamin H (biotin), 100 to 1,000 μg ephemeranthone, 1000 to 10,000 μg demethoxycurcumin, 100 to 1,000 μg zopfinol, and 100 to 1,000 μg (+)-fargesin per 100 mg of extract.

In another embodiment, the turmeric extract comprises 25 to 500 μg bamosamine, 100 to 3,000 μg bisdemethoxycurcumin, 500 to 75,000 μg curcumin, 10 to 500 μg eugenol, 0200 to 3,000 μg methoxycoumarin, 100 to 2,000 μg elijopyrone D, and 100 to 5,000 μg vitamin H (biotin) per 100 mg of extract.

In another embodiment, the turmeric extract comprises 25 to 500 μg bamosamine, 100 to 3,000 μg bisdemethoxycurcumin, 500 to 75,000 μg curcumin, 50 to 1,000 μg lysine, 100 to 3,000 μg methoxycoumarin, 10 to 500 μg α-phenylindol, 50 to 1,000 μg 3,4-dihydroscopoletin, 50 to 5,000 μg 11-epileontidane, 10 to 500 μg methoxyflavanone, 50 to 500 μg aconitic acid triethyl ester, 50 to 500 μg 5,7-dimethoxyflavanone, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

In another embodiment, the turmeric extract comprises 25 to 500 μg bamosamine, 100 to 3,000 μg bisdemethoxycurcumin, 500 to 75,000 μg curcumin, 10 to 500 μg eugenol, 0200 to 3,000 μg methoxycoumarin, 100 to 2,000 μg elijopyrone D, 100 to 5,000 μg vitamin H (biotin), 50 to 1,000 μg lysine, 100 to 3,000 μg methoxycoumarin, 10 to 500 μg α-phenylindol, 50 to 1,000 μg 3,4-dihydroscopoletin, 50 to 5,000 μg 11-epileontidane, 10 to 500 μg methoxyflavanone, 50 to 500 μg aconitic acid triethyl ester, 50 to 500 μg 5,7-dimethoxyflavanone, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

Another aspect of the invention relates to a pharmaceutical composition comprising any of the aforementioned extracts and a pharmaceutically acceptable carrier.

Another aspect of the invention relates to a pharmaceutical composition that blocks β-amyloid plaque aggregation. In other embodiments, the invention relates to a pharmaceutical composition that blocks β-amyloid plaque secretion.

Another aspect of the invention relates to a pharmaceutical composition that blocks β-amyloid plaque accumulation in brain tissue. In other embodiments, the invention relates to a pharmaceutical composition that inhibits hyper-phosphorylation of tau protein in vivo in brain tissues. In some embodiments, the pro-inflammatory response is suppressed and the cytokines IL-2 and IL-4 are increased in brain tissues.

Another aspect of the invention relates to a method of treating or preventing a neurodegenerative disorder in a subject in need thereof comprising administering to the subject a therapeutically effecting amount of any of the aforementioned extracts. In some embodiments, the neurodegenerative disorder is Alzheimer's disease. In other embodiments, the neurodegenerative disorder is dementia.

Another aspect of the invention relates to a turmeric extract prepared by a process comprising: extracting turmeric with supercritical carbon dioxide in a supercritical extraction vessel, wherein the extraction vessel has a pressure from 300 to 800 bar and temperature of 50 to 100° C.

In another embodiment, the turmeric extract is prepared by a process comprising: extracting turmeric with a mixture of water and ethanol.

### BRIEF DESCRIPTION OF THE DRAWINGS

FIG. 1 depicts the DART TOF-MS of turmeric Extract 1 with the X-axis representing the mass-to-charge (m/z) ratio and the Y-axis representing relative abundance (RA) of the chemicals present.

FIG. 2 depicts the DART TOF-MS of turmeric Extract 2 with the X-axis representing the mass-to-charge (m/z) ratio and the Y-axis representing relative abundance (RA) of the chemicals present.

FIG. 3 depicts the DART TOF-MS of turmeric Extract 2 with the X-axis representing the mass-to-charge (m/z) ratio and the Y-axis representing relative abundance (RA) of the chemicals present.

FIG. 4 depicts the effects of the turmeric extracts and curcuminoid standards on Aβ₁₋₄₂ aggregation as determined by the thioflavin T assay. The Aβ₁₋₄₂ peptide (25 μM) was incubated at 37° C. alone and in the presence of turmeric extracts (Extract 1, Extract 2 and

Extract 3), as well as curcuminoid standards for comparison, at varying concentrations as indicated for 120 h. All experiments were carried out in Tris-HCL buffer (pH 7.4). Data are represented as percent aggregation based off the relative fluorescence units of Aβ₁₋₄₂ peptide incubated alone (n=3). Extract 1=-▪-, Extract 2=-▴-, Extract 3=- [symbol] -, Curcumin standard=-▾-, Demethoxycurcumin standard=-□-, Bisdemethoxycurcumin standard=-♦-, tetrahydrocurcumin standard=-+-.

FIG. 5 depicts the inhibition of Aβ generation in cultured neuronal cells. Aβ_{1-40, 42} peptides were analyzed in conditioned media from SweApp N2a cells by ELISA (n=3 per condition). Data are represented as percentage of Aβ_{1-40, 42} peptides secreted 8 h after Extract 1, Extract 2, and Extract 3, and the curcuminoid standards relative to control. Both turmeric Extract 1 and Curcumin inhibit Aβ generation in cultured neuronal cells, however, tetrahydrocurcumin increased Aβ generation while Extract 2 displayed no significant effect. Extract 1=-▪-, Extract 2=-▴-, Extract 3=- [symbol] -, Curcumin standard=-▾-, Demethoxycurcumin standard=-♦-, Bisdemethoxycurcumin standard=- [symbol] -, tetrahydrocurcumin standard=-+-.

FIG. 6 depicts the inhibition of amyloid plaque accumulation by oral administration of Extract 1 and THC to Tg2576 mice where reduction Aβ deposition is observed with both treatments (A). Image analysis of micrographs from Aβ antibody (4G8) stained sections reveals that plaque burdens were significantly reduced throughout the entorhinal cortex and hippocampus (P<0.01, P<0.05; B) with Extract 1 and to a much lesser degree with THC.

FIG. 7 depicts the marked inhibition of both soluble (A; 1% Triton, 40%) and insoluble forms (B; 5 M guanidine, 20%) of Aβ_{1-40, 42} compared to the THC-treated animals and the untreated control animals which were not significantly different.

FIG. 8 depicts the soluble fractions of phosphorylated tau detected in the homogenates of the treatment groups and their control mice by both Ser^{199/220} and AT8 antibodies. The Tg2576 mice orally treated with either Extract I and THC show decreased phosphorylated tau protein based on Western blotting (A), with Extract 1 being most effective (P<0.01, FIG. 8B). Extract 1 reduces hyper-phosphorylation by 82% compared to control (B), while THC treated mice showed only a ca. 40% reduction in tau phosphorylation over untreated control animals (B).

FIG. 9 depicts the IL-4 to IL-2 cytokine profile of Extract 1- and THC-treated Tg2576 mice. Following sacrifice, primary cultures of splenocytes were established from the mice and stimulated for 24 hours with anti-CD3 antibody. The levels of IL-4 to IL-2 cytokines (A) in Extract 1-treated mice was significantly increased (P<0.001; ca. double that of control animals) compared to untreated control animals and THC-treated animals. The ratio of IL-4 to IL-2 cytokines levels (B) for Extract 1 were 1.1, while for the THC IL-4:IL-2 ratio was 0.8 which was not significantly differ from that found for control animals.

### DETAILED DESCRIPTION OF THE INVENTION

### Definitions

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “extract” refers to a product prepared by extraction. The extract may be in the form of a solution in a solvent, or the extract may be a concentrate or essence which is free of, or substantially free of solvent. The extract also may be formulated into a pharmaceutical composition or food product, as described further below. The term extract may be a single extract obtained from a particular extraction step or series of extraction steps, or the extract also may be a combination of extracts obtained from separate extraction steps. Such combined extracts are thus also encompassed by the term “extract.”

As used herein, “feedstock” generally refers to raw plant material, comprising whole plants alone, or in combination with one or more constituent parts of a plant comprising leaves, roots, including, but not limited to, main roots, tail roots, and fiber roots, stems, bark, leaves, berries, seeds, and flowers, wherein the plant or constituent parts may comprise material that is raw, dried, steamed, heated or otherwise subjected to physical processing to facilitate processing, which may further comprise material that is intact, chopped, diced, milled, ground or otherwise processed to affected the size and physical integrity of the plant material. Occasionally, the term “feedstock” may be used to characterize an extraction product that is to be used as feed source for additional extraction processes.

As used herein, the term “fraction” means the extraction composition comprising a specific group of chemical compounds characterized by certain physical, chemical properties or physical or chemical properties.

A “patient,” “subject” or “host” to be treated by the subject method may be a primate (e.g. human), bovine, ovine, equine, porcine, rodent, feline, or canine.

The term “pharmaceutically-acceptable salts” is art-recognized and refers to the relatively non-toxic, inorganic and organic acid addition salts of compounds, including, for example, those contained in compositions of the present invention.

The term “synergistic” is art recognized and refers to two or more components working together so that the total effect is greater than the sum of the components.

The term “treating” is art-recognized and refers to curing as well as ameliorating at least one symptom of any condition or disorder.

The term “effective amount” as used herein refers to the amount necessary to elicit the desired biological response. As will be appreciated by those of ordinary skill in this art, the effective amount of a drug may vary depending on such factors as the desired biological endpoint, the drug to be delivered, the composition of the encapsulating matrix, the target tissue, etc.

As used herein, the term “inhibitor” refers to molecules that bind to enzymes and decrease their activity. The binding of an inhibitor can stop a substrate from entering the enzyme's active site and/or hinder the enzyme from catalyzing its reaction. Inhibitor binding is either reversible or irreversible. Irreversible inhibitors usually react with the enzyme and change it chemically. These inhibitors modify key amino acid residues needed for enzymatic activity. Reversible inhibitors bind non-covalently and different types of inhibition are produced depending on whether these inhibitors bind the enzyme, the enzyme-substrate complex, or both.

As used herein, the term “Amyloid” refers to any fibril, plaque, seed, or aggregate that has the characteristic cross-β sheet structure.

As used herein, the term “Amyloidogenic precursor” refers to a protein or peptide that upon incubation under appropriate conditions will form amyloid fibrils or plaques.

As used herein, the term “Amyloid fibril” refers to long ribbons of amyloid ˜10 nm in diameter and >100 nm in length. Most often observed in vitro.

As used herein, the term “Amyloid plaque” refers to the form of amyloid most often found in vivo—often comprised of aggregated amyloid fibrils.

As used herein, the term “Amyloid protofibril/filament” refers to a species of amyloid smaller in diameter (3-6 nm) and length (<100 nm) than typical for amyloid fibrils, thought to be a possible direct precursor to amyloid fibrils perhaps through lateral aggregation.

As used herein, the term “Amyloid seed” (or template) refers to a species of a critical size or structure that rapidly elongates to form larger amyloid species possibly by providing a proper scaffold for amyloid assembly

As used herein, the term “Amyloidogenic oligomer” refers to a small aggregate of precursor that is smaller than the critical “seed” size but still may have some of the structural characteristics of amyloid.

As used herein, the term “Amyloidogenic fold” refers to a structure of the precursor that must be accessed prior to amyloidogenic aggregation, thought to retain substantial secondary structure possibly including some of the native fold. It could be related to a misfolded or molten globule structure.

As used herein, the term “Tau” refers to a class of microtubule-associated proteins that are abundant in neurons in the central nervous system. Tau proteins interact with tubulin to stabilize microtubules and promote tubulin assembly into microtubules. Tau has two ways of controlling microtubule stability: isoforms and phosphorylation. Six tau isoforms exist in brain tissue, and they are distinguished by their number of binding domains.

As used herein, the term “Tau phosphorylation” or “Tau hyper-phosphorylation” refers phosphorylation of tau via a host of kinases. For example, when PKN, a serine/threonine kinase is activated, it phosphorylates tau, resulting in disruption of microtubule organization. Hyper-phosphorylation of the tau protein (tau inclusions), however, can result in the self-assembly of tangles of paired helical filaments and straight filaments, which are involved in the pathogenesis of Alzheimer's disease and other tau pathologies.

As used herein, the term “Folded state” refers to the native (functional) state of the precursor.

As used herein, the term “Folding intermediate” refers to a partially folded or misfolded structure of the precursor. These partially folded structures are potentially the same as or precursors to amyloidogenic folds.

As used herein, the term “Denatured state” refers to the unfolded state of the precursor.

As used herein, the term “Unstructured aggregate” refers to the completely or partially denatured proteins tend to aggregate non-specifically without forming a particular structural motif.

As used herein, the term “AD” refers to Alzheimer's Disease which is a degenerative and terminal disease that is the most common form of dementia. AD has been identified as a protein misfolding disease due to the accumulation of abnormally folded amyloid beta protein in the brains of AD patients.

As used herein, the term “Amyloid” refers to any fibril, plaque, seed, or aggregate that has the characteristic cross-β sheet structure.

As used herein, the term “APP” refers to the amyloid precursor protein which is an integral membrane protein expressed in many tissues and concentrated in the synapses of neurons. Its primary function is not known, though it has been implicated as a regulator of synapse formation and neural plasticity. APP is best known and most commonly studied as the precursor molecule whose proteolysis generates amyloid beta, a 39- to 42-amino acid peptide whose amyloid fibrillar form is the primary component of amyloid plaques found in the brains of Alzheimer's disease patients.

As used herein, the term “Secretase” refers to protease enzymes that “snip” pieces off a longer protein that is embedded in the cell membrane, and they includes α-, β-, and γ-secretases. Secretases act on the amyloid precursor protein (APP) to cleave the protein into three fragments. Sequential cleavage by β-secretase (BACE) and γ-secretase produces the amyloid-β peptide fragment that aggregates into clumps called “plaques” in the brains of AD patients. If α-secretase acts on APP first instead of BACE, no amyloid-β is formed because α-secretase recognizes a target protein sequence closer to the cell surface than BACE.

As used herein, the term “Blood brain barrier” or “BBB” refers to the separation of circulating blood and cerebrospinal fluid (CSF) maintained by the choroid plexus in the central nervous system. Endothelial cells restrict the diffusion of microscopic objects (e.g., bacteria) and large or hydrophillic molecules into the CSF, while allowing the diffusion of small hydrophobic molecules (O₂, hormones, CO₂, small molecules). Cells of the barrier actively transport metabolic products such as glucose across the barrier with specific proteins

The compounds in the extracts of the present invention may be present in the form of pharmaceutically-acceptable salts derived from inorganic or organic acids. By “pharmaceutically-acceptable salt” is meant those salts that are, within the scope of sound medical judgment, suitable for use in contact with the tissues of humans and lower animals without undue toxicity, irritation, and allergic response, and are commensurate with a reasonable benefit/risk ratio. Pharmaceutically-acceptable salts are well known in the art. For example, S. M. Berge, et al. describe pharmaceutically-acceptable salts in J Pharm Sci, 1977, 66:1-19. The salts may be prepared in situ during the final isolation and purification of the compounds of the invention or separately by reacting a free base function with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates, such as dimethyl, diethyl, dibutyl and diamyl sulfates; long-chain halides such as decyl, lauryl, myristyl and stearyl chlorides, bromides and iodides; or arylalkyl halides, such as benzyl and phenethyl bromides and others. Water- or oil-soluble or -dispersible products are thereby obtained.

Examples of acids that may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, and citric acid.

The present invention includes all salts and all crystalline forms of such salts. Basic addition salts can be prepared in situ during the final isolation and purification of compounds of this invention by combining a carboxylic acid-containing group with a suitable base such as the hydroxide, carbonate, or bicarbonate of a pharmaceutically-acceptable metal cation or with ammonia or an organic primary, secondary, or tertiary amine. Pharmaceutically-acceptable basic addition salts include cations based on alkali metals or alkaline earth metals such as lithium, sodium, potassium, calcium, magnesium, and aluminum salts, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as cancer, an infectious disease, or other medical disease or condition, is well understood in the art, and includes administration of a composition which reduces the frequency of, or delays the onset of, symptoms of a medical condition in a subject relative to a subject which does not receive the composition. Thus, prevention of an infection includes, for example, reducing the number of diagnoses of the infection in a treated population versus an untreated control population, and/or delaying the onset of symptoms of the infection in a treated population versus an untreated control population.

### Extracts

One aspect of the invention relates to extracts of turmeric comprising an enriched amount of certain compounds having activity against neurological diseases, such as Alzheimer's disease. In certain embodiments, the extract has been optimized for use for treatment of neurological diseases. For example, the extract may inhibit Aβ aggregation, inhibit Aβ formation, or both, and may inhibit deposition of amyloids in brain tissue, and inhibit hyper-phosphorylation of tau and fibril formation.

The extracts can also be described in terms micrograms of individual compound per 100 mg of extract. Thus, another aspect of the invention relates to a turmeric extract comprising at least one compound selected from the group consisting of 25 to 500 μg bamosamine, 25 to 750 μg echinaxanthol, 100 to 3,000 μg bisdemethoxycurcumin, 50 to 500 μg daphniyunnine E and 500 to 75,000 μg curcumin per 100 mg of extract.

In another embodiment, the extract further comprises at least one compound selected from the group consisting of 50 to 500 μg decadienal/santolina epoxide, 10 to 500 μg eugenol, 0200 to 3,000 μg methoxycoumarin, 100 to 2,000 μg elijopyrone D, 100 to 5,000 μg vitamin H (biotin), and 50 to 500 μg epierythrostominol per 100 mg of extract.

In another embodiment, the turmeric extract further comprises at least one compound selected from the group consisting 50 to 1,000 μg lysine, 100 to 3,000 μg methoxycoumarin, 10 to 500 μg ethoxycoumarin, 10 to 500 μg α-phenylindol, 50 to 1,000 μg 3,4-dihydroscopoletin, 50 to 5,000 μg vasicinone, 50 to 5,000 μg 11-epileontidane, 10 to 500 μg methoxyflavanone, 50 to 500 μg aconitic acid triethyl ester, 50 to 500 μg 5,7-dimethoxyflavanone, 10 to 1,000 μg piperine, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

One aspect of the invention relates to a turmeric extract comprising at least one compound selected from the group consisting of 0.01 to 1% by weight of bamosamine, 0.01 to 5% by weight of echinaxanthol, 0.1 to 10% by weight of bisdemethoxycurcumin, 0.01 to 1% by weight of daphniyunnine E and 0.1 to 80% by weight of curcumin. In another embodiment, the extract comprises at least one of 0.05 to 0.3% by weight of bamosamine, 0.05 to 0.5% by weight of echinaxanthol, 0.2 to 2% by weight of bisdemethoxycurcumin, 0.05 to 0.2% by weight of daphniyunnine E, and 0.5 to 50% by weight of curcumin.

In some embodiments, the turmeric extract further comprises at least one compound selected from the group consisting of 0.01 to 2% by weight decadienal/santolina epoxide, 0.01 to 1% by weight of eugenol, 0.1 to 5% by weight of methoxycoumarin, 0.05 to 5% by weight of elijopyrone D, 0.1 to 10% by weight of vitamin H (biotin), and 0.05 to 2% by weight of epierythrostominol

In some embodiments, the extract comprises one or more of the aforementioned compounds, and in other embodiments, the extract comprises all of the aforementioned compounds. For example, the aforementioned turmeric extracts can comprise at least one of 0.01 to 0.5% by weight of bamosamine, 0.01 to 0.5% by weight of echinaxanthol, 0.1 to 2% by weight of bisdemethoxycurcumin, 0.01 to 0.3% by weight of daphniyunnine E, 0.5 to 50% by weight of curcumin, 0.05 to 0.5% by weight decadienal/santolina epoxide, 0.01 to 0.3% by weight of eugenol, 0.3 to 2% by weight of methoxycoumarin, 0.1 to 1% by weight of elijopyrone D, 0.1 to 5% by weight of vitamin H (biotin), and 0.05 to 1% by weight of epierythrostominol

In another embodiment, the extract comprises 0.05 to 0.3% by weight of bamosamine, 0.05 to 0.5% by weight of echinaxanthol, 0.2 to 2% by weight of bisdemethoxycurcumin, 0.05 to 0.2% by weight of daphniyunnine E, 0.5 to 50% by weight of curcumin, 0.1 to 0.5% by weight decadienal/santolina epoxide, 0.02 to 0.2% by weight of eugenol, 0.5 to 2% by weight of methoxycoumarin, 0.2 to 1% by weight of elijopyrone D, 0.2 to 3% by weight of vitamin H (biotin), and 0.1 to 0.5% by weight of epierythrostominol

In another embodiment, any of the aforementioned extracts further comprise at least one compound selected from the group consisting of 0.01 to 2% by weight of lysine, 0.1 to 5% by weight of methoxycoumarin, 0.01 to 1% by weight of ethoxycoumarin, 0.01 to 1% by weight of α-phenylindol, 0.01 to 2% by weight of 3,4-dihydroscopoletin, 0.01 to 5% by weight of vasicinone, 0.01 to 5% by weight of 11-epileontidane, 0.01 to 1% by weight of methoxyflavanone, 0.01 to 1% by weight of aconitic acid triethyl ester, 0.01 to 1% by weight of 5,7-dimethoxyflavanone, 0.01 to 2% by weight of piperine, 0.1 to 2% by weight of ephemeranthone, 0.1 to 2% by weight of neohesperidose, 0.1 to 15% by weight of demethoxycurcumin, 0.1 to 2% by weight of zopfinol, 0.01 to 1% by weight of dehydroagastanol, and 0.1 to 2% by weight of (+)-fargesin. The extract may comprise one or more of these compounds, or it may comprise all of these compounds.

For example, in another embodiment, the aforementioned extracts comprise at least compound selected from the group consisting of 0.01 to 0.5% by weight of bamosamine, 0.01 to 0.5% by weight of echinaxanthol, 0.1 to 2% by weight of bisdemethoxycurcumin, 0.01 to 0.3% by weight of daphniyunnine E, 0.5 to 50% by weight of curcumin, 0.01 to 1% by weight of lysine, 0.1 to 3% by weight of methoxycoumarin, 0.01 to 0.5% by weight of ethoxycoumarin, 0.01 to 0.5% by weight of α-phenylindol, 0.05 to 1% by weight of 3,4-dihydroscopoletin, 0.05 to 3% by weight of vasicinone, 0.05 to 3% by weight of 11-epileontidane, 0.05 to 1% by weight of methoxyflavanone, 0.01 to 0.5% by weight of aconitic acid triethyl ester, 0.05 to 0.5% by weight of 5,7-dimethoxyflavanone, 0.01to 1% by weight of piperine, 0.1 to 1% by weight of ephemeranthone, 0.1 to 1% by weight of neohesperidose, 0.1 to 10% by weight of demethoxycurcumin, 0.1 to 1% by weight of zopfinol, 0.01 to 0.5% by weight of dehydroagastanol, and 0.1 to 1% by weight of (+)-fargesin.

In some embodiments, the aforementioned extract comprises 0.05 to 0.3% by weight of bamosamine, 0.05 to 0.5% by weight of echinaxanthol, 0.2 to 2% by weight of bisdemethoxycurcumin, 0.05 to 0.2% by weight of daphniyunnine E, 0.5 to 50% by weight of curcumin, 0.05 to 0.5% by weight of lysine, 0.5 to 2% by weight of methoxycoumarin, 0.02 to 0.3% by weight of ethoxycoumarin, 0.02 to 0.3% by weight of α-phenylindol, 0.1 to 1% by weight of 3,4-dihydroscopoletin, 0.1 to 3% by weight of vasicinone, 0.03 to 0.5% by weight of 11-epileontidane, 0.05 to 0.3% by weight of methoxyflavanone, 0.1 to 0.5% by weight of aconitic acid triethyl ester, 0.1 to 0.5% by weight of 5,7-dimethoxyflavanone, 0.2 to 1% by weight of piperine, 0.2 to 1% by weight of ephemeranthone, 0.2 to 1% by weight of neohesperidose, 0.2 to 10% by weight of demethoxycurcumin, 0.2 to 1% by weight of zopfinol, 0.05 to 0.3% by weight of dehydroagastanol, and 0.2 to 1% by weight of (+)-fargesin.

### Pharmaceutical Compositions

Another aspect of the invention relates to pharmaceutical compositions comprising any of the aforementioned turmeric extracts and at least one pharmaceutically acceptable carrier are provided.

Compositions of the disclosure comprise extracts of turmeric in forms such as pastes, powders, oils, liquids, suspensions, solutions, ointments, or other forms, comprising, one or more fractions or sub-fractions to be used as dietary supplements, nutraceuticals, or such other preparations that may be used to prevent or treat various conditions. The extracts can be processed to produce such consumable items, for example, by mixing them into a food product, in a capsule or tablet, or providing the paste itself for use as a dietary supplement, with sweeteners or flavors added as appropriate. Accordingly, such preparations may include, but are not limited to, turmeric extract preparations for oral delivery in the form of tablets, capsules, lozenges, liquids, emulsions, dry flowable powders and rapid dissolve tablets. The turmeric extracts may advantageously be formulated into a suppository or lozenge for vaginal administration

Compositions can be in the form of a paste, resin, oil, powder or liquid. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hyroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

Dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

### Methods of Treatment

The present invention also relates in part to methods of treating or preventing neurological disorders in a subject in need thereof comprising administering to the subject an effective amount of any of the aforementioned extracts or pharmaceutical compositions. In some embodiments, the neurodegenerative disease is associated with amyloid plaques. In some embodiments, the method of treatment prevents the aggregation of amyloid plaques, while in other embodiments, the method of treatment prevents the formation of amyloids. In other embodiments, the method of treatment prevents amyloid plaque deposition in brain tissues, while in other embodiments, the method of treatment prevents hyper-phosphorylation of tau and fibril formation in brain tissues. In some embodiments, the neurological disorder is Alzheimer's disease, while in others it is dementia.

While not being bound by any particular theory, it is believed that the aforementioned extracts prevent amyloid aggregation, amyloid production or both, and prevent amyloid plaque deposition, tau hyper-phosphorylation and fibril formation in neurological tissues. For example, the extracts contain compounds that inhibit amyloid aggregation. In some embodiments, the extracts contain compounds that inhibit amyloid precursor protein (APP) secretion. In other embodiments, the extracts inhibit tau hyper-phosphorylation and fibril formation in brain tissues.

### Methods of Preparing Turmeric Extracts

Another aspect of the invention relates to supercritical extraction methods of making turmeric extracts. The turmeric may be provided in the form of a ground turmeric root, for example, ground Curcuma longa L. The turmeric root is loaded into a supercritical carbon dioxide extractor and subjected to the extraction. In one embodiment, the method comprises extracting turmeric with supercritical carbon dioxide in a supercritical extraction vessel, wherein the extraction vessel has a pressure from 300 to 800 bar and temperature of 50 to 100° C. In some embodiments, the pressure is about 300, 400, 500, 600, 700 or 800 bar. In other embodiments, the pressure is 500 to 700 bar, while in other embodiments, the pressure is about 600 bar. In some embodiments, the temperature of the extraction is 60 to 100° C., while in other embodiments, the temperature is 70 to 90° C. In other embodiments, the temperature is about 80 to 85° C., and in other embodiments, the temperature is about 85° C., such as 83° C. In some embodiments, the aforementioned pressure and temperature are maintained for about 60 to 280 min, or about 100 to 150 min, or about 120 min.

In some embodiments, the extraction apparatus further comprises three separators in series. The method thus can further comprise separating the supernatant from the extraction step at about 100 to 200 bar and 35 to 100° C. In another embodiment, the separator has a pressure of about 120 or 150 bar. In some embodiments, the temperature of the separator is about 50 to 75° C., or about 55 to 70° C., or about 56 or 67° C.

In another embodiment, a turmeric extract is prepared by extracting turmeric with a water and/or ethanol. For example, the method comprising providing turmeric root, which may be ground into a powder, and extracting with water, or aqueous ethanol, or 100% ethanol. In some embodiments, the aqueous ethanol comprises more than 10%, 20%, 30%, 40%, 50%, 60%, 70% 80% or 90% ethanol. In some embodiments, the aqueous ethanol is 50 to 95% ethanol, or 80 to 90% ethanol. In other embodiments, the aqueous ethanol is about 85% ethanol. In other embodiments, the extraction is carried out with 100% ethanol.

In some embodiments, the extraction is carried out at a temperature of 10 to 90° C. In other embodiments, the extraction is carried out at 20 to 60° C., for example, about 25° C., or 40° C. In some embodiments the extraction is carried out for 1 to 6 h, 1 to 4 h, or about 2 h. In some embodiments, the extraction is carried out in more than one stage, for example 2, 3 or more stages.

The method may further comprise filtering the resulting slurry, and evaporating the water, ethanol, or aqueous ethanol. After extraction, the slurry was filtered through Fisher brand P4 filter paper with pore size of 4-8 μm and centrifuged at 2000 rpm for 20 min. The supernatants were collected and evaporated to dryness at 50° C. under vacuum. Extract 2 was prepared using 85% (v/v) ethanol at 40° C. for 2 h. Extract 3 was prepared by using Extract 2 as the feedstock and extracting with 100% (USP) ethanol at 25° C. for 1 h.

### Exemplification

The disclosure now being generally described, it will be more readily understood by reference to the following examples, which are included merely for purposes of illustration of certain aspects and embodiments of the disclosure, and are not intended to limit the disclosure.

### Materials and Methods

A. Turmeric (Curcuma longa) Feedstock

Ground turmeric (Curcuma longa L.) roots were obtained from commercial sources. The species was verified by the supplier as Curcuma longa L.

### B. Turmeric Extraction Procedure

1. Super-Critical CO₂ Extractions

Super Critical CO₂ (SCCO₂) extraction was conducted on customized supercritical fluid extraction and fractionation systems. This system is comprised of two main 24-L extraction vessels, three 20 L separation, CO₂ pump, additive pump, electrical heat exchanges, fluid-cooled condenser, CO₂ accumulator, mass flow meter, and chiller. The system is controlled by two national instruments compact field-point processors (CFP-2020 and CFP-200). National Instrument Labview RT (real time) runs on these processors using a custom software application.

Ground turmeric root was extracted using super critical CO₂. The compressed CO₂ extracted the essential oil and other lipophilic substances including curcuminoids. The solution remaining in the extractor was processed stage-wise by precipitations of the extracts using different solvent pressure and temperature in three stages in separate separators. In a typical experiment, the temperature and pressure of the extractor were set at 83° C. and 600 bar respectively with a solvent/feed ratio of 150. The conditions for the three separators were 150 bar and 67° C. for separator 1; 130 bar and 56° C. for separator 2; and 65 bar and 28° C. for separator 3. Extract 1 was prepared from the first separator at 130 bar and 56° C. Extract 3 was prepared by extracting Extract 2 at 25° C. with 100% USP ethanol and collecting the supernatant.

TABLE 1
Specific extraction conditions for Turmeric Extracts
1, 2, and 3 incorporating super critical CO₂, ethanol
and water to generate the extracts used to inhibit Aβ aggregation
and APP secretion.

| Extract Number | Extraction Conditions |
|---|---|
| 1 | SFT extraction: 150 bar; 67° C. |
| 2 | Turmeric feedstock: 85% Ethanol; 40° C. |
| 3 | 100% Ethanol; 25° C. extraction of Extract 2 |

### C. HPLC Analysis of Extracts

A Shimadzu High Performance Liquid Chromatographic LC-10AVP system equipped with LC10ADVP pump with SPD-M 10AVP photo diode array detector was used for sample analysis. The samples were analyzed using a reversed phase Jupiter C18 column (250×4.6 mm I. D., 5μ, 300 Å) (Phenomenex). The mobile phase consisted of A (0.5% acetic, v/v) and B (acetonitrile). The gradient was programmed as follows: 0-30 min, solvent B increased linearly from 30 to 36%, 30 to 40 min, B linearly from 36 to 95%, and then 40-44 min, B linearly from 9 to 30% and held for 1 min. The detector was set at 423 nm. Methanol stock solutions of 3 standards (BDMC, DMC and curcumin) were diluted to yield a range of concentrations. The retention times of BDMC, DMC and curcumin were 23.7, 25.8, and 28.1 min, respectively, as measured at 423 nm. A linear fit ranging from 0.01 to 20 μg was found. The regression equations and correlation coefficients were as follows: BDMC: Area/100=64410×C (μg), R²=0.9998 (N=7); DMC: Area/100=117367×C (μg), R²=0.9998 (N=7); curcumin: Area/100=63930×C (μg), R²=0.9998 (N=7). The contents of the reference standards in each sample were calculated by interpolation from the corresponding calibration curves based on the peak area.

### D. DART TOF-MS Characterization of Extract

A DART™AccuTOF-mass spectrometer (JMS-T100LC; Jeol USA, Peabody, Mass.) was used for chemical analysis of the turmeric extracts and was executed in positive ion mode [M+H]⁺. The needle voltage was set to 3500V, heating element to 300° C., electrode 1 to 150V, electrode 2 to 250V, and helium gas flow to 3.98 L/min. For the mass spectrometer, the following settings were loaded: orifice 1 set to 20V, ring lens voltage set to 5V, and orifice 2 set to 5V. The peak voltage was set to 1000V in order to give peak resolution beginning at 100 m/z. The microchannel plate detector (MCP) voltage was set at 2550V. Calibrations were performed internally with each sample using a 10% (w/v) solution of PEG 600 (Ultra Chemical, North Kingston, R.I.) that provided mass markers throughout the required mass range 100-1000 m/z. Calibration tolerances were held to 10 mmu. Turmeric extracts were introduced into the DART helium plasma using the closed end of a borosilicate glass melting point capillary tube until a signal was achieved in the total-ion chromatogram (TIC). The next sample was introduced when the TIC returned to baseline levels. Candidate molecular formulae were identified using elemental composition and isotope matching programs in the Jeol MassCenterMain Suite software (JEOL USA, Peabody, Mass.).

### E. Identification of Compounds in Turmeric Extracts

The accurate masses determined by DART TOF-MS analysis of the turmeric extracts were used to identify known compounds in the extracts by searching against a HerbalScience accurate mass proprietary database of natural products. These known compounds were confirmed by searching the Dictionary of Natural Products (CRC Press, Boca Raton, Fla.) and the NIST/EPA/NIH (NIST, Gaithersburg, Md.) mass spectral database. The compounds in turmeric likely to be contributing to the observed in vitro biological activity were determined using proprietary algorithms for the correlation of exact masses and extract activity.

### F. Amyloid Aggregation Assay

The presence of Aβ₁₋₄₂ fibers was monitored in solution by thioflavin T fluorescence as previously described (S. A. Moore, T. N. Huckerby, G. L. Gibson, N. J. Fullwood, S. Turnbull, B. J. Tabner, O. M. El-Agnaf and D. Allsop, 2004. Both the D-(+) and L-(−) enantiomers of nicotine inhibit Abeta aggregation and cytotoxicity, Biochemistry. 43:819-826; H. LeVine, 3rd, 1993. Thioflavine T interaction with synthetic Alzheimer's disease beta-amyloid peptides: detection of amyloid aggregation in solution, Protein Sci. 2:404-410). Briefly, triplicate 20 μL samples of Aβ₁₋₄₂ [25 μM] in 50 mM Tris-HCl buffer (pH 7.4) were removed after incubation of the peptide solution in the presence or absence of optimized turmeric extracts 1, 2 and 3 or the curcuminoid standards Cur, DMC, BDMC and THC; Chromadex, Irvine, Calif.) at concentrations from 0 to 30 μg/mL for up to 120 h at 37° C. These peptide solutions were each added to 100 μL of 10 μM thioflavin T (Sigma) in 50 mM glycine/NaOH buffer (pH 9.0) in a black-walled 96-well plate for 30 min at room temperature before that the characteristic change in fluorescence was monitored (excitation at 450 nm and emission at 482 nm) following binding of thioflavin T to the amyloid fibers at 25° C. by using a Molecular Devices SPECTRAmax GEMINI plate reader. Triplicate samples were scanned three times before and immediately after the addition of the peptide solutions. Results show the mean value of the triplicate samples±the difference between those mean values.

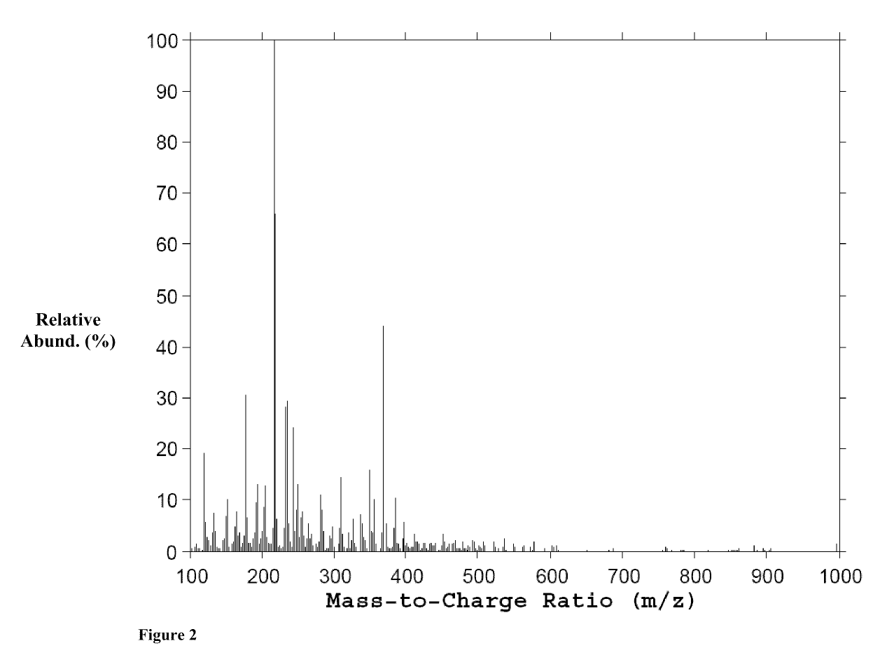

The Aβ aggregation assays were carried out with the synthetic Aβ₁₋₄₂ peptide incubated with the extracts (Extract 1, Extract 2, and Extract 3) or the curcuminoid standards (Curcumin=Cur, Demthoxycurcumin=DMC, bisdemethoxycurcumin=BDMC, and tetrahydrocurcumin=THC) at varying concentrations from 0 to 30 μg mL⁻¹ at 120 h (FIG. 2) with aggregation being monitored by the thioflavin T method. The thioflavin T method detects mainly mature β-pleated sheet amyloid fibers. FIG. 2 shows that Extract 1, Cur, THC, BDMC, and DMC are all effective, while Extract 2 is not an effective inhibitor of Aβ₁₋₄₂ aggregation. The 50% inhibition (IC₅₀) values ranged from 5-10 μg ml⁻¹ at 20 μM Aβ₁₋₄₂ concentration. Among the curcuminoids, DMC was the least effective inhibitor of Aβ₁₋₄₂ aggregation.

### G. Aβ₁₋₄₂ Secretion ELISA Assay

Conditioned media were collected and analyzed at a 1:1 dilution using the method as previously described (J. Tan, T. Town, F. Crawford, T. Mori, A. DelleDonne, R. Crescentini, D. Obregon, R. A. Flavell and M. J. Mullan, 2002. Role of CD40 ligand in amyloidosis in transgenic Alzheimer's mice, Nat. Neurosci. 5:1288-1293) and values were reported as percentage of Aβ₁₋₄₂ secreted relative to control in SweAPP N2a cells. Quantification of total Aβ species was performed according to published methods (P. Marambaud, H. Zhao and P. Davies, 2005. Resveratrol promotes clearance of Alzheimer's disease amyloid-beta peptides, J Biol Chem. 280:37377-37382; D. F. Obregon, K. Rezai-Zadeh, Y. Bai, N. Sun, H. Hou, J. Ehrhart, J. Zeng, T. Mori, G. W. Arendash, D. Shytle, T. Town and J. Tan, 2006. ADAM10 activation is required for green tea (−)-epigallocatechin-3-gallate-induced alpha-secretase cleavage of amyloid precursor protein, J Biol Chem. 281:16419-16427). Briefly, 6E10 (capture antibody) was coated at 2 μg/mL in phosphate buffered saline (PBS; pH 7.4) into 96-well immunoassay plates overnight at 4° C. The plates were washed with 0.05% (v/v) Tween-20 in PBS five times and blocked with blocking buffer (PBS with 1% BSA, 5% [v/v] horse serum) for 2 h at room temperature.

Conditioned medium or Aβ standards were added to the plates and incubated overnight at 4° C. Following 3 washes, biotinylated antibody, 4G8 (0.5 μg/mL in PBS with 1% [w/v] BSA) was added to the plates and incubated for 2 h at room temperature. After 5 washes, streptavidin-horseradish peroxidase (1:200 dilutions in PBS with 1% BSA) was added to the 96-wells for 30 min at room temperature.

Tetramethylbenzidine (TMB) substrate was added to the plates and incubated for 15 minutes at room temperature. A 50 μL aliquot of stop solution (2 N N₂SO₄) was added to each well of the plates to top the reaction. The optical density of each well was determined immediately on a microplate reader at 450 nm. The Aβ levels were expressed as a percentage of control (conditioned medium from untreated N2a SweAPP cells).

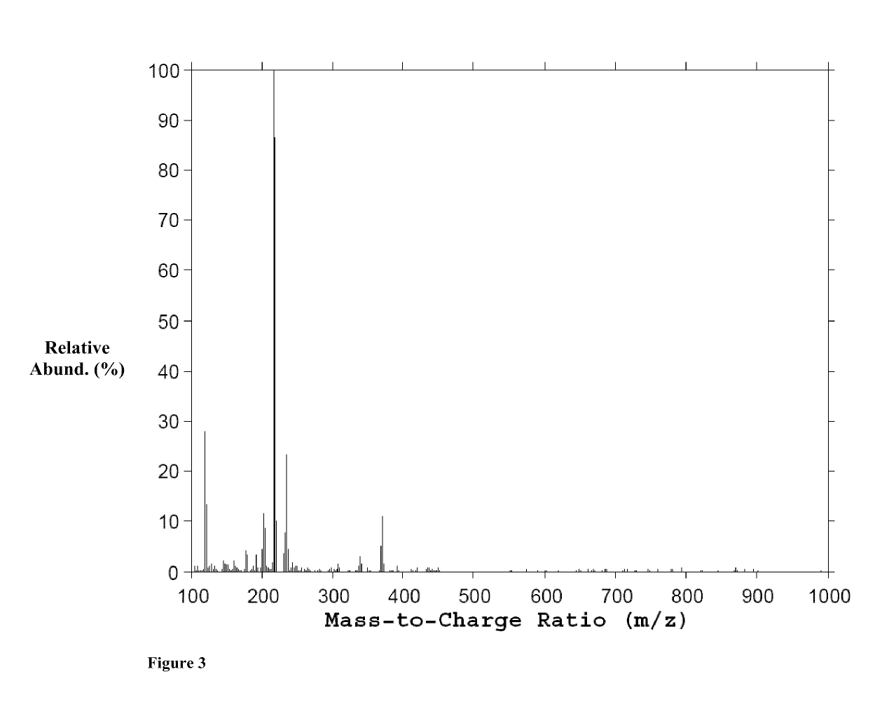

In order to compare the effects of turmeric extracts (Extract 1, Extract 2, and Extract 3), and the curcuminoid standards (Cur, DMC, BDMC and THC) on APP (Amyloid Precursor Protein) cleavage, the SweAPP N2a cells were treated with a concentration-range of 3-30 μg/ml of each compound or extract for 12 h (FIG. 3). The Aβ_{1-40, 42} peptides were analyzed in conditioned media from SweAPP N2a cells by ELISA (n=3 for each condition). Data are represented as percentage of Aβ_{1-40, 42} peptides secreted in 12 h after turmeric extracts or the curcuminoid standards were added relative to a control. As shown in FIG. 3, Extract 1 and the curcumin standard significantly reduce Aβ generation (both Aβ₁₋₄₀ and Aβ₁₋₄₂ peptides) in SweAPP N2a cells in a concentration-dependent manner. In contrast Extract 3 (97% turmerones) and two curcuminoids, DMC and BDMC, showed only limited inhibition of Aβ generation (ca. 10%), while Extract 2, enriched in turmerones over Extract 1, showed no inhibition. Interestingly, THC stimulated Aβ secretion from SweAPP N2a cells.

### H. Curcuminoid and Turmeric Extract Interaction Matrices

Interaction matrices were designed following the methods of Delaney et al. (W. E. I. Delaney, H. Yang, M. D. Miller, C. S. Gibbs and S. Xiong, 2004. Combinations of adefovir with nucleoside analogs produce additive antiviral effects against hepatitis B virus in vitro, Antimicrobial Agents and Chemotheraphy. 48:3702-3710) to address the possible antagonistic, synergistic and/or additive effects of the different extracts and the individual curcuminoids when combined with Extract 1 and the other extracts and the individual curcuminoids on inhibition of Aβ₁₋₄₂ aggregation. Matrices included a range of concentrations of extracts and the curcuminoids that were combined in equal portions ranging from 0 amounts of each to amounts that exceed the IC₁₀₀ values. These combinations were then evaluated in the in vitro Aβ₁₋₄₂ aggregation assay, and experimental and theoretical IC₅₀ values were determined If the experimental IC₅₀ values in the combined samples decreased beyond a simple additive effect reflected in the theoretical IC₅₀ value, the combined effects were synergistic, and if the IC₅₀ values increased the combined effects were antagonistic (C. A. Fairbanks and G. L. Wilcox, 1999. Spinal antinociceptive synergism between morphine and clonidine persists in mice made acutely or chronically tolerant to morphine, J. Pharm. Exp. Ther. 288:1107-1116).

### I. Alzheimer Pathologies in Brain of Tg2576 Mice

1. Reagents

Anti-human amyloid-β antibodies 4G8 and 6E10 were obtained from Signet Laboratories (Dedham, Mass., USA) and Biosource International (Camarillo, Calif., USA), respectively. VectaStain Elite™ ABC kit was purchased from Vector Laboratories (Burlingame, Calif., USA). Aβ_{1-40, 42} ELISA kits were obtained from IBL-American (Minneapolis, Minn., USA). Anti-phospho-tau antibodies including Ser^{199/220} and AT8 were purchased from Innogenetics (Alpharetta, Ga., USA). Turmeric Extract 1 was used along with commercial THC (Chromadex, Irvine, Calif.).

2. In Vivo Animal Treatments

The Tg2576 mice, which are engineered to develop AD within ca. 6 months after birth, were purchased from Taconic (Germantown, N.Y.). For oral administration of extracts, a total of 60 (30 female/30 male) Tg2576 mice with a B6/SJL background were employed. Beginning at 8 months of age, Tg2576 treatment mice were administered optimized turmeric Extract 1 and THC in NIH31 chow (0.07% in NIH31 chow, 167 mg/kg/day) or NIH31 chow alone (Control) for 6 months [n=20 (10 female/10 male)]. All mice were sacrificed at 14 months of age for analyses of Aβ levels and Aβ load in the brain according to previously described methods (J. Tan, T. Town, F. Crawford, T. Mori, A. DelleDonne, R. Crescentini, D. Obregon, R. A. Flavell and M. J. Mullan, 2002. Role of CD40 ligand in amyloidosis in transgenic Alzheimer's mice, Nat. Neurosci. 5:1288-1293). Animals were housed and maintained in the College of Medicine Animal Facility at the University of South Florida (USF), and all experiments were in compliance with protocols approved by the USF Institutional Animal Care and Use Committee.

3. Immunohistochemistry

Mice were anesthetized with isofluorane and transcardially perfused with ice-cold physiological saline containing heparin (10 U/mL). Brains were rapidly isolated and quartered using a mouse brain slicer (Muromachi Kikai Co., Tokyo, Japan). The first and second anterior quarters were homogenized for ELISA and Western blot analysis as described above, and the third and fourth posterior quarters were used for microtome or cryostat sectioning. Brains were then fixed in 4% (w/v) paraformaldehyde in PBS at 4° C. overnight and routinely processed in paraffin. Five coronal sections from each brain (5-μm thickness) were cut with a 150-μm interval. Sections were routinely de-paraffinized and hydrated in a graded series of ethanol prior to pre-blocking for 30 min at ambient temperature with serum-free protein block (Dakocytomation, Glostrup, Denmark). The Aβ immunohistochemical staining was performed using anti-human amyloid-β antibody (clone 4G8, 1:100) in conjunction with the VectaStain Elite™ ABC kit coupled with diaminobenzidine substrate. The 4G8-positive Aβ deposits were examined under bright-field using an Olympus BX-51 microscope. Quantitative image analysis (conventional “Aβ burden” analysis) was routinely performed for 4G8 immuno-hitochemistry. Data are reported as percentage of immunolabeled area captured (positive pixels) divided by the full area captured (total pixels).

4. Image Analysis

Quantitative image analysis (conventional “Aβ burden” analysis) was performed for 4G8 immunohitochemistry and Congo red histochemistry for brains from Tg2576 mice orally administrated optimized turmeric Extract 1, THC, or NIH31 control chow. Images were obtained using an Olympus BX-51 microscope and digitized using an attached MagnaFire™ imaging system (Olympus, Tokyo, Japan). Briefly, images of five 5-μm sections (150 μm apart) through each anatomic region of interest (hippocampus or cortical areas) were captured and a threshold optical density was obtained that discriminated staining form background. Manual editing of each field was used to eliminate artifacts. Data are reported as percentage of immunolabeled area captured (positive pixels) divided by the full area captured (total pixels). Quantitative image analysis was performed by a single examiner (JZ) blinded to sample identities.

5. Aβ ELISA

Mouse brains were isolated under sterile conditions on ice and placed in ice-cold lysis buffer (20 mM Tris, pH 7.5, 150 mM NaCl, 1 mM EDTA, 1 mM EGTA, 1% (v/v) Triton X-100, 2.5 mM sodium pyropgosphate, 1 mM β-glycerolphosphate, 1 mM Na₃VO₄, 1 μg/mL leupeptin, 1 mM PMSF) as previously described (J. Tan, T. Town, F. Crawford, T. Mori, A. DelleDonne, R. Crescentini, D. Obregon, R. A. Flavell and M. J. Mullan, 2002. Role of CD40 ligand in amyloidosis in transgenic Alzheimer's mice, Nat. Neurosci. 5:1288-1293). Brains were then sonicated on ice for approximately 3 min, allowed to stand for 15 min at 4° C., and centrifuged at 15,000 rpm for 15 min. The Aβ_{1-40, 42} species were detected by acid extraction of brain homogenates in 5 M guanidine buffer (K. Johnson-Wood, M. Lee, R. Motter, K. Hu, G. Gordon, R. Barbour, K. Khan, M. Gordon, H. Tan, D. Games, I. Lieberburg, D. Schenk, P. Seubert and L. McConlogue, 1997. Amyloid precursor protein processing and Aβ42 deposition in a transgenic mouse model of Alzheimer's disease, Proc. Natl. Acad. Sci. USA. 94:1550-1555) followed by a 1:10 dilution in lysis buffer. Soluble Aβ_{1-40, 42} were directly detected in brain homogenates prepared with lysis buffer described above by a 1:10 dilution. Protein levels of homogenate samples were all normalized by BCA protein assay prior to dilution. The Aβ_{1-40, 42} was quantified in these samples using the Aβ_{1-40, 42} ELISA kits in accordance with the manufacturer's instructions, except that standards included 0.5 M guanidine buffer in some cases.

6. Western Blot Analysis

Brain homogenates were obtained as previously described above. For tau analysis, aliquots corresponding to 100 μg of total protein was electrophoretically separated using 10% Tris gels. Electrophoresed proteins were then transferred to nitrocellulose membranes (Bio-Rad, Richmond, Calif., USA), washed in double distilled H₂O, and blocked for 1 h at ambient temperature in Tris-buffered saline (TBS) containing 5% (w/v) non-fat dry milk. After blocking, membranes were hybridized for 1 h at ambient temperature with various primary antibodies. Membranes were then washed 3 times for 5 min each in double distilled H₂O and incubated for 1 h at ambient temperature with the appropriate HRP-conjugated secondary antibody (1:1,000, Pierce Biotechnology, Rockford, Ill.). All antibodies were diluted in TBS containing 5% (w/v) of non-fat dry milk. Blots were developed using the luminol reagent (Pierce Biotechnology, Rockford, Ill.). Densitometric analysis was done as previously described using a FluorS Multiimager with Quantity One™ software (BioRad, Hercules, Calif.) (K. Rezai-Zadeh, D. Shytle, N. Sun, T. Mori, H. Hou, D. Jeanniton, J. Ehrhart, K. Townsend, J. Zeng, D. Morgan, J. Hardy, T. Town and J. Tan, 2005. Green tea epigallocatechin-3-gallate (EGCG) modulates amyloid precursor protein cleavage and reduces cerebral amyloidosis in Alzheimer transgenic mice, J. Neurosci. 25:8807-8814).

7. Cytokine ELISA

As described in the previous studies (J. Tan, T. Town, D. Paris, T. Mori, Z. Suo, F. Crawford, M. P. Mattson, R. A. Flavell and M. Mullan, 1999. Microglial activation resulting from CD40-CD40L interaction after beta-amyloid stimulation, Science. 286:2352-2355; J. Tan, T. Town, M. Saxe, D. Paris, Y. Wu and M. Mullan, 1999. Ligation of microglial CD40 results in p44/42 mitogen-activated protein kinase-dependent TNF-alpha production that is opposed by TGF-beta 1 and IL-10, J. Immunol. 163:6614-6621) cell cultured media were collected for measurement of cytokines by commercial cytokine ELISA kits. In parallel, cell lysates were prepared for measurement of total cellular protein. Data will be represented as ng/mg total cellular protein for each cytokine production. Cytokines were quantified using commercially available ELISAs (BioSource International, Inc., Camarillo, Calif.) that allow for detection of IL-2 and IL-4. Cytokine detection will be carried out according to the manufacturer's instruction.

8. Statistical Analysis

All data were normally distributed; therefore, in instances of single mean comparisons, Levene's test for equality of variances followed by t-test for independent samples was used to assess significance. In instances of multiple mean comparisons, analysis of variance (ANOVA) was used, followed by post-hoc comparison using Bonferonni's method. Alpha levels were set at 0.05 for all analyses. The statistical package for the social sciences release 10.0.5 (SPSS Inc., Chicago, Ill.) or Statistica© was used for all data analysis.

### Results

### A. HPLC Analysis of Turmeric Extracts

The HPLC analysis results of Extracts 1 and 2 are show in Table 2. The curcuminoid fraction can be purified to greater than 75% curcuminoids by weight.

TABLE 2
The processing yield and purify obtained by supercritical carbon
dioxide extraction and fractionation.

| | | Total | BDMC | DMC | | |
|---|---|---|---|---|---|---|
| | Total | Curcuminoid | Purity | Purity | Curcumin | Total |
| Extract | Yield | Yield (%) | (%) | (%) | Purity (%) | (%) |
| 1 | 2.30 | 1.75 | 3.7 | 12.0 | 60.5 | 76.2 |
| 2 | 2.25 | 1.53 | 3.1 | 6.6 | 58.2 | 67.9 |

### B. DART TOF-MS Identification of Compounds in Turmeric Extracts

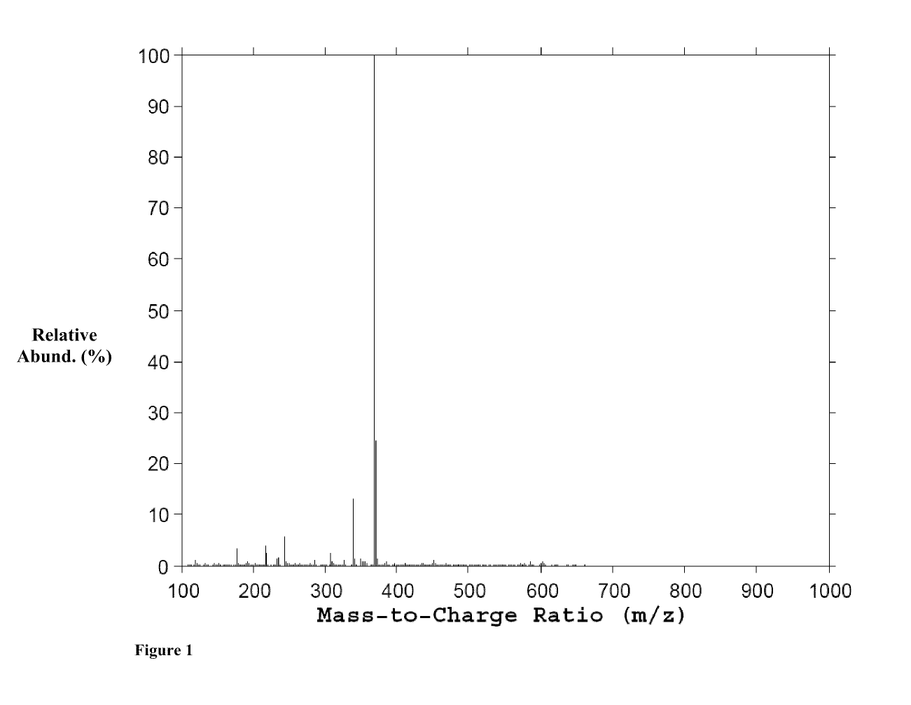

FIG. 1-3 show the DART TOF-MS spectra of turmeric Extracts 1-3. The exact mass is plotted along the X axis while the relative abundances is plotted on the Y axis for each identified mass based on searching exact mass databases. Tables 3-5 provide a summary of the identified compounds (measured mass and relative abundances) present in the various extracts.

TABLE 3
Compounds present in Extract 1 as determined by DART
TOF-MS analysis and utilization of a searchable database.

| | | Relative |
|---|---|---|
| | Measured | Abundance |
| Compound Name | Mass | (%) |
| 3-Methyl-2-butenoic acid, 9CI | 101.0537 | 0.0611 |
| 4-Fluorobutanoic acid, 9CI | 107.0489 | 0.0755 |
| 1,2-Dimethylbenzene, 9CI | 107.0861 | 0.1088 |
| 1,3,6-Octatriene | 109.1005 | 0.1918 |
| 2-Methyl-1-cyclopentene-1- | 111.0791 | 0.0697 |
| carboxaldehyde | | |
| 3-Aminobutanoic acid, 9CI; (±)-form: N,N- | 113.1093 | 0.0545 |
| Di-Me, nitrile | | |
| 1-Ethyl-2-methylcyclopentane, 9CI | 113.1323 | 0.0468 |
| 2-Methylaminoacetic acid: Et ester | 118.0838 | 0.0393 |
| 2,3-Diaminopropanoicacid; (R)-form: N3- | 119.0857 | 2.8917 |
| Me | | |
| Isopropylbenzene, 8CI | 121.1004 | 0.8744 |
| 5-Ethyl-2-methylpyridine | 122.1031 | 0.1353 |
| Benzoic acid, 9CI, USAN | 123.0485 | 0.4018 |
| (2-Hydroxyethyl)dimethylsulfoxonium(1+) | 124.0499 | 0.1039 |
| 2-Chloro-2-propenoic acid, 9CI: Chloride | 124.9514 | 0.0328 |
| 6-Methyl-3,5-heptadien-2-one, 9CI; (E)- | 125.0980 | 0.3406 |
| (?)-form | | |
| 2-Amino-4-hydroxypyrimidine; 1H-form: | 126.0739 | 0.0970 |
| 1-Me | | |
| 1-Isothiocyanato-2-methylbutane | 130.0718 | 0.1350 |
| 3-Methyl-1-butylamine: N-Propyl | 130.1601 | 0.1275 |
| 2-Methyl-3,4-piperidinediol, 9CI | 132.1030 | 0.2013 |
| 2,4-Diaminopentanoicacid | 133.1023 | 0.5673 |
| Noractinidine | 134.1049 | 0.0330 |
| Glycerol, INN: Tri-Me ether | 135.1000 | 1.1388 |
| 2-Acetylpyridine: Hydrazone | 136.0936 | 0.2139 |
| 4-Methylbenzoic acid, 9CI | 137.0644 | 0.6023 |
| 2-Hydroxybenzoic acid, 9CI | 139.0490 | 0.1294 |
| 2-Pentylfuran | 139.1137 | 0.0962 |
| 2-Ethyl-2,3-dihydro-6-methyl-4H-pyran- | 141.0952 | 0.0637 |
| 4-one, 9CI | | |
| 2-Butanol, 9CI; (±)-form: 2-Methyl-2- | 143.1047 | 0.0505 |
| propenoyl | | |
| 2-Amino-3-hydroxymethyl-3-pentenoic | 146.0887 | 0.1033 |
| acid, 8CI | | |
| Lysine | 147.0475 | 0.3509 |
| 2-Hydroxybenzoic acid, 9CI: Et ether, | 148.0664 | 0.0601 |
| nitrile | | |
| 2-Amino-5-hydroxyhexanoic acid | 148.0944 | 0.0379 |
| Diethanolamine: N-Propyl | 148.1256 | 0.0647 |
| 3-(2-Pyrrolidinyl)pyridine, 9CI | 149.1147 | 0.3713 |
| 4-Methylbenzoic acid, 9CI: Methylamide | 150.0973 | 0.1067 |
| 2,6-Decadien-4-yn-1-ol, 8CI | 151.1145 | 1.0494 |
| decadienal | 152.0596 | 0.0646 |
| 2-Vinyl-1,3,5-benzenetriol | 153.0564 | 1.5477 |
| Cytosine: 4-N—Ac | 154.0617 | 0.1172 |
| 4-Hydroxy-2-cyclopenten-1-one, 9CI; (R)- | 155.1002 | 0.0804 |
| form: tert-Butyl ether | | |
| 5-Methyl-2,3-hexanedione, 9CI: Dioxime | 159.1179 | 0.2135 |
| Putreanine | 161.1321 | 0.2833 |
| Boschniakine; (R)-form | 162.0950 | 0.0551 |
| Safrole | 163.0784 | 0.7425 |
| 2-Amino-4,5-dihydroxy-4-methylpentanoic | 164.0871 | 0.1941 |
| acid | | |
| Benzylamine, 8CI: N,N-Di-Et | 164.1409 | 0.1130 |
| Eugenol | 165.0823 | 1.1729 |
| 2-Amino-2-phenylpropanoic acid | 166.0793 | 0.2534 |
| 3-Hydroxy-p-mentha-1,8-dien-7-al | 167.1077 | 0.4549 |
| 10-Hydroxy-4-pinanone | 169.1223 | 0.5367 |
| 2-Aminoethanol, 9CI: N,N-Di-Et, 2- | 172.1375 | 0.0947 |
| propenoyl | | |
| 1,4-Diguanidinobutane | 173.1421 | 0.2373 |
| 2-Amino-4-ethylidenepentanedioic acid | 174.0694 | 0.1020 |
| Indospicine | 174.1269 | 0.0972 |
| 1,4-Diamino-1,4-dideoxyglucitol, 9CI; Di- | 174.2019 | 0.0331 |
| NH-form: N1,N3,N3′-Tri-Me | | |
| N-Methyltryptamine | 175.1234 | 0.4572 |
| 4-Amino-1-phenyl-1-penten-3-one, 9CI | 176.1139 | 0.1650 |
| methoxycoumarin | 177.0572 | 4.6319 |
| Bamosamine | 178.0614 | 0.7068 |
| Nicotine, BSI, ISO; (S)-form: 1′-N-Oxide | 179.1280 | 1.0037 |
| Tecomanine | 180.1371 | 0.2478 |
| Herbipoline | 181.1023 | 0.2353 |
| 1-Methyl-9H-carbazole, 9CI | 182.1052 | 0.0793 |
| Dendrobates Alkaloid 181A | 182.1980 | 0.0354 |
| Dihydro-5-(5-hydroxy-1,3-hexadienyl)-2(3H)- | 183.0987 | 0.2387 |
| furanone | | |
| 6-Tridecene | 183.2071 | 0.0479 |
| 3,7-Dimethyl-1-(methylthio)-2,6- | 185.1294 | 0.0931 |
| octadiene; (E)-form | | |
| Dihydro-5-(4-hydroxy-4-methylpentyl)-2(3H)- | 187.1388 | 0.1382 |
| furanone | | |
| 1H-Indole-3-methanamine: 3-N—Ac | 189.1066 | 0.3611 |
| 1-Isothiocyanato-6-(methylthio)hexane, | 190.0660 | 0.5452 |
| 9CI | | |
| ethoxycoumarin | 191.1075 | 0.4881 |
| 2-Amino-2-deoxyribose; D-form: N—Ac | 192.0829 | 0.0790 |
| Elijopyrone D | 193.0950 | 1.4449 |
| Alpha-phenylindol | 194.0919 | 0.4085 |
| 3,4-Dihydroscopoletin | 195.0741 | 1.9529 |
| Asterubin | 196.0822 | 0.1828 |
| 4-(3-Aminopropyl)-1,2-benzenediol, 9CI: | 196.1414 | 0.0612 |
| Di-Me ether | | |
| Elaeokanine A; (S)-form: 1′-Alcohol (1′S- | 196.1766 | 0.0503 |
| ?) | | |
| 2-Acetyl-4,4,6-trimethyl-1,3- | 197.1148 | 0.2143 |
| cyclohexanedione, 9CI | | |
| 2,8,10-Pentadecatriene-4,6-diyne, 8CI | 199.1424 | 0.3884 |
| Tetradecane | 199.2390 | 0.0588 |
| a-Amino-4-carboxy-3-furanpropanoic acid, | 200.0568 | 0.0117 |
| 9CI; (S)-form | | |
| 2-Pentylquinoline, 9CI | 200.1531 | 0.0237 |
| Dodecanoicacid, 9CI: Amide | 200.1928 | 0.0163 |
| 1,3,5,7-Cadinatetraene, 8CI | 201.1663 | 0.5966 |
| 2-Aminoheptanoic acid; (±)-form: N-Di- | 202.1745 | 0.1105 |
| Me, Et ester | | |
| vasicinone | 203.1810 | 1.3030 |
| 3,5-Cadinadiene | 205.1994 | 1.0940 |
| Dendrobates Alkaloid 203: Dihydro (?) | 206.1992 | 0.1937 |
| 6-Deoxyglucose, 9CI, 8CI; β-L-Pyranose- | 207.1253 | 0.4150 |
| form: 3-Me, Et glycoside | | |
| Felinine | 208.0934 | 0.1760 |
| Xanthostemone | 209.1192 | 0.2416 |
| Triacsin B: 6,7,8,9-Tetrahydro | 210.1633 | 0.1203 |
| Antibiotic A 41-89; Antibiotic A 41-89I | 211.1352 | 0.1839 |
| Linderazulene: 2,3-Dihydro | 213.1368 | 0.1925 |
| Hexahydro-7a-hydroxy-3H-pyrrolizin-3- | 214.1389 | 0.1026 |
| one, 9CI; (±)-form: (1-Ethoxyethyl) ether | | |
| Tsitsikammafuran | 215.1453 | 0.6656 |
| Decanedioic acid, 9CI: Amide-Me ester | 216.1532 | 0.3333 |
| Verboccidentafuran | 217.1606 | 15.1547 |
| N-Deacetylkuanoniamine D; (Z)-form: 2- | 218.1635 | 2.5572 |
| Methylpropylamide | | |
| 1,3,5-Cadinatrien-10-ol; (7a H,10a)-form | 219.1764 | 9.9976 |
| Sedamine | 220.1796 | 1.5273 |
| 11-Epileontidane | 221.1948 | 0.9368 |
| 2-Hydroxybenzoic acid, 9CI: | 223.1056 | 0.1192 |
| Tetrahydrofurfuryl ester | | |
| Murexine | 225.1460 | 0.1731 |
| 5,10-Pentadecadien-1-ol | 225.2225 | 0.0594 |
| 1-Hexadecene | 225.2582 | 0.0754 |
| 2,4-Dialkylthiazoles; 4-Ethyl-2- | 226.1645 | 0.0291 |
| octylthiazole, 9CI | | |
| 9-Tetradecenoic acid; (E)-form | 227.2043 | 0.0652 |
| Faramol | 229.1272 | 0.1207 |
| Tetradecanoic acid, 9CI | 229.2186 | 0.1037 |
| Verboccidentafuran: 2-Oxo | 231.1412 | 0.6720 |
| 2-Aminoheptanedioic acid, 8CI; (±)-form: | 232.1502 | 0.2478 |
| Di-Et ester | | |
| Verboccidentafuran: 4a,5a-Epoxide | 233.1546 | 4.2778 |
| Hypusine | 234.1718 | 1.0224 |
| 9-Hydroxy-4,10(14)-oplopadien-3-one | 235.1709 | 4.4484 |
| 3,10(14),11-Germacratriene-1,9-diol | 237.1914 | 0.8266 |
| Dendrobates Alkaloid 237E | 238.2078 | 0.1550 |
| 1,2,3,4-Tetrahydro-5,6,7- | 239.1521 | 0.0702 |
| trihydroxyisoquinoline: 6,7-Di-Me ether, | | |
| N,N-di-Me | | |
| 10-Propyl-5,9-tridecadien-1-ol, 9CI; (Z)- | 239.2365 | 0.3007 |
| form | | |
| Spherophysine: N4′-Ac | 241.2064 | 0.1106 |
| Pearlmycin, 9CI | 241.2546 | 0.0656 |
| Arabinitol, 9CI; D-form: 1-Benzyl | 243.1260 | 0.1167 |
| 2,6,10-Trimethyldodecanoic acid | 243.2351 | 0.2225 |
| Echinaxanthol | 245.0848 | 3.6745 |
| Mycosporin-Gly | 246.0904 | 0.5018 |
| 8-Hydroxy-1,4,7(11)-guaiatrien-12,8-olide | 247.1390 | 0.5988 |
| 5-(2-Hydroxyethyl)-4-methylthiazole: | 248.0741 | 0.0394 |
| Benzoyl | | |
| 4-Amino-4,6-dideoxy-3-C- | 248.1430 | 0.1839 |
| methylmannose; β-D-Pyranose-form: Me | | |
| glycoside, N-Me, N—Ac | | |
| 6-Hydroxy-4,11(13)-eudesmadien-12,8- | 249.1534 | 1.1985 |
| olide | | |
| 4-Amino-4-deoxyglucuronic acid; a-D- | 250.0899 | 0.0206 |
| Pyranose-form: Me glycoside, N—Ac | | |
| Fungerin: N1-Me | 250.1701 | 0.2732 |
| Thienodolin | 250.9980 | 0.0137 |
| 1-(3,5-Dichloro-4-methoxyphenyl)-1,2- | 251.0336 | 0.0161 |
| propanediol, 9CI | | |
| 1-Hydroxy-5,11(13)-eudesmadien-12-oic | 251.1679 | 1.9864 |
| acid | | |
| 2′-Deoxyadenosine, 9CI, 8CI | 252.1104 | 0.0133 |
| Arglecin | 252.1836 | 0.4117 |
| 1-Hydroxy-4(15),11(13)-eudesmadien-12- | 253.1809 | 0.4413 |
| oic acid; (1a,7β)-form: 11,13-Dihydro | | |
| 2-Amino-11,15-hexadecadien-3-ol | 254.2446 | 0.2370 |
| methoxyflavanone | 255.2309 | 0.9934 |
| Dendrobates Alkaloid 253B | 256.2584 | 0.2042 |
| 8-Methylpentadecanoic acid, 9CI | 257.2538 | 1.1754 |
| 2-(2-Hydroxybutyl)-6-(2-hydroxypentyl)- | 258.2490 | 0.1746 |
| 1-methylpiperidine | | |
| Aconitic acid, triethyl ester | 259.1898 | 0.4752 |
| Obliquin; (S)-form: 5′-Hydroxy | 261.0792 | 0.0691 |
| 1,3,6,9-Nonadecatetraene | 261.2495 | 0.0929 |
| 8-Methyl-8-azabicyclo[3.2.1]octane-3,6- | 262.1411 | 0.0914 |
| diol, 9CI; (3R,6R)-form: 3-Benzoyl | | |
| 10-Hydroxy-1,3,5-cadinatrien-15-oic acid; | 263.1569 | 0.3726 |
| (7β,10a OH)-form: Me ester | | |
| 3-(Dimethylaminomethyl)-5- | 265.1549 | 0.8363 |
| hydroxyindole: a r,N-Dimethoxy, Me | | |
| ether | | |
| Brevicolline; (S)-form | 266.1627 | 0.2333 |
| 3,8-Dihydroxy-4(15),9,11(13)- | 267.1629 | 0.3911 |
| germacratrien-12,6-olide; (3β,6a,8a,9 E)- | | |
| form: 11a,13-Dihydro | | |
| 9-Octadecenal | 267.2661 | 0.1585 |
| Brevicarine | 268.1866 | 0.0748 |
| 14-Pentadecenoic acid: Et ester | 269.2439 | 0.5115 |
| 3-Methylpentadecanoic acid, 9CI; (±)- | 271.2566 | 0.1613 |
| form: Me ester | | |
| 2,4-Diamino-5,6-dihydroxypyrimidine: 5- | 275.1025 | 0.0445 |
| O-Arabinopyranoside | | |
| 1,7,16-Hexadecanetriol; (?)-form | 275.2594 | 0.1803 |
| 8-Methyl-8-azabicyclo[3.2.1]octane-3,6- | 276.1563 | 0.0442 |
| diol, 9CI; (3R,6R)-form: 3-O- | | |
| Phenylacetyl | | |
| 3,18-Heneicosadiene-1,8,10,20-tetrayne; | 277.1995 | 0.1024 |
| (Z,Z)-form | | |
| 7-Hydroxy-14,15-dinor-8(17)-labden-13- | 279.2282 | 0.2727 |
| one | | |
| 2,4-Tetradecadienoic acid, 9CI; (2E,4E)- | 280.2618 | 0.2999 |
| form: 2-Methylpropylamide | | |
| 1,13-Dihydroxy-4-oxo-2-pseudoguaien- | 281.1488 | 0.1313 |
| 12,6-olide | | |
| 9,12-Octadecadienoic acid, 9CI; (9E,12Z)- | 281.2570 | 0.4880 |
| form | | |
| 10-Octadecenoic acid; (E)-form: Amide | 282.2817 | 1.6838 |
| 5-(10- | 283.2752 | 1.2196 |
| Aminoundecyl)hexahydropyrrolo[2,1-b]oxazole | | |
| 5,7-dimethoxyflavanone | 285.2822 | 0.6186 |
| 1-Piperoylpiperidine | 286.1542 | 0.2849 |
| piperine | 286.2766 | 0.1071 |
| 2-Amino-3-octadecanol | 286.3173 | 0.0389 |
| Komaroine | 287.1530 | 0.0280 |
| N-(3-Hydroxy-1-oxocyclopent-2-en-2-yl)- | 290.0959 | 0.0592 |
| 3-(4-hydroxy-3-methoxyphenyl) | | |
| 3-Docosene-1,11,13,15,21-pentayne, 9CI; | 291.2184 | 0.0877 |
| (Z)-form: Dihydro | | |
| Lasiodiplodin; (R)-form: 6-Oxo, O-de-Me | 293.1465 | 0.4738 |
| 10(14)-Aromadendrene-2,3,4-triol; | 295.1952 | 0.3786 |
| (1a,2a,3a,4a,5a,6β,7β)-form: 2-Ac | | |
| 2-Amino-4,8,10-octadecatriene-1,3-diol | 296.2630 | 0.0550 |
| Dendrobates Alkaloid 295 | 296.3014 | 0.0253 |
| 3-O-Methylgalactose, 9CI, 8CI; a-D- | 297.1303 | 0.7214 |
| Pyranose-form: Me glycoside, 4,6-O- | | |
| benzylidene | | |
| Cassine; (−)-form | 298.2832 | 0.0873 |
| 1,10:4,5-Diepoxy-3,6,8-trihydroxy-11- | 299.1524 | 0.0869 |
| germacren-9-one; (1β,3a,4a,5a,6a,8a,10β)- | | |
| form | | |
| 4-Oxooctadecanoic acid | 299.2580 | 0.0636 |
| Palmidrol, INN | 300.2861 | 0.0540 |
| 2′,3′,4,4′,6,7-Hexahydroxyisoflavan | 307.0906 | 0.0437 |
| 2-Hydroxydodecanoic acid; (R)-form: | 307.2310 | 0.1893 |
| Benzyl ester | | |
| Aspergillomarasmine A | 308.1099 | 0.1263 |
| Bisdemethoxycurucmin | 309.1141 | 4.9305 |
| 1H-Indole-5,6-diol, 9CI: N,O6-Disulfo | 309.9685 | 0.0139 |
| Mescaline succinimide: 3-Hydroxy | 310.1218 | 1.0271 |
| 1,1,3-Tribromo-3-chloro-1-propene | 310.8100 | 0.0173 |
| Antibiotic A11-99-1 | 310.8531 | 0.0190 |
| Ephemeranthone | 311.1358 | 2.1723 |
| Diiodoaceticacid: Amide | 311.8442 | 0.0299 |
| 4,5-Dibromo-1H-pyrrole-2,3-dicarboxylic | 311.8980 | 0.0156 |
| acid | | |
| 2-Amino-2-deoxygalactose, 9CI, 8CI; a-D- | 312.1430 | 0.3535 |
| Pyranose-form: Benzyl glycoside, N—Ac | | |
| 5-[(4-Hydroxyphenyl)ethenyl]-2-(3- | 313.1501 | 0.4893 |
| methyl-1-butenyl)-1,3-benzenediol: 3′- | | |
| Hydroxy | | |
| Dehydroisolongistrobine: Dihydro | 314.1527 | 0.1025 |
| Acetylleucylargininal; L-DL-form | 314.2125 | 0.0298 |
| Prosopine‡: 11′-Ketone | 314.2772 | 0.0814 |
| Flourensianol: Tigloyl | 315.1643 | 0.0535 |
| 12,13-Dihydroxy-9-octadecenoicacid | 315.2508 | 0.1253 |
| 18-Hydroxy-19-trachylobanoic acid | 319.2216 | 0.0635 |
| 2,7,11-Cembratriene-4,10-diol; (1S,2E,4R, | 319.2585 | 0.0857 |
| 7E,10R,11Z)-form: 10-Ketone, 4-Me | | |
| ether | | |
| Rutaecarpine: 7β,8a-Dihydroxy | 320.1124 | 0.5601 |
| 13-Docosenoic acid; (E)-form: Nitrile | 320.3311 | 0.0887 |
| Methyl β-D-glucopyranoside: 2,3,4-Tri-Ac | 321.1097 | 0.0765 |
| Bavachromene | 323.1324 | 0.0884 |
| Cneorumchromene G | 325.1467 | 0.3510 |
| Tetrahydrothalifendine | 326.1484 | 0.0399 |
| neohesperidose | 327.1505 | 0.9427 |
| Cryptostyline I; (R)-form | 328.1552 | 0.2287 |
| 9,10-Dihydroxyocta-decanoicacid; (9R, | 331.2853 | 0.0986 |
| 10R)-form: Me ester | | |
| 3-(12-Phenyl-8-dodecenyl)phenol | 337.2599 | 0.1095 |
| 1H-Indol-3-ylacetyl-myo-inositol | 338.1198 | 1.1032 |
| Demethoxycurcumin | 339.1242 | 17.7967 |
| Stylopine, 9CI; (S)-form: 13β-Hydroxy | 340.1267 | 4.6197 |
| Zopfinol | 341.1428 | 1.8617 |
| Daphniyunnine E | 342.1648 | 0.4361 |
| Dehydroagastanol | 343.1765 | 0.3292 |
| (2,4-Dimethoxy-3- | 344.2291 | 0.1265 |
| prenylcinnamoyl)piperidine | | |
| 5,6-Dibromotryptamine: Nb,Nb-Di-Me | 344.9922 | 0.0443 |
| Chondriol | 345.0506 | 0.0322 |
| Gelsedine, 9CI: 14R-Hydroxy | 345.1830 | 0.0507 |
| Mescaline isocitrimide lactone | 350.1162 | 2.4237 |
| Epierythrostominol | 351.1235 | 0.5982 |
| 2-Amino-3-(3,5-dibromo-4- | 351.9602 | 0.0394 |
| hydroxyphenyl)propanoic acid; (S)-form: | | |
| 4-Me ether | | |
| Eritadenine; (2R,3R)-form: 2,3-Di-Ac, | 352.1332 | 0.4259 |
| Me ester | | |
| Estra-1,3,5(10)-triene-3,17-diol; 17β-form: | 353.1457 | 0.5513 |
| 3-O-Sulfate | | |
| Hackelidine: 7-Ac | 354.1486 | 0.1139 |
| Isostemonidine | 354.2219 | 0.0432 |
| 1,3,9-Trihydroxy-10-prenylpterocarpan: 1- | 355.1530 | 1.5153 |
| Me ether | | |
| Rutacridone: 1′,2′-Dihydro, 1′-hydroxy, | 356.1494 | 0.4390 |
| 2′-methoxy | | |
| Xanthoascin | 357.1595 | 0.1825 |
| Glycerol 1-alkanoates; Glycerol 1-(9Z- | 357.3081 | 0.1765 |
| octadecenoate) | | |
| 3,14,17,21-Tetrahydroxypregn-5-en-20- | 365.2341 | 0.0606 |
| one; (3β,14β,17βOH)-form | | |
| Collinusin | 367.1181 | 0.5467 |
| Karnamicin A1: 1″-Deoxy | 368.1244 | 4.1351 |
| Curcumin | 369.1332 | 100.0000 |
| Tecleaverdoornine: Ac | 370.1371 | 25.7916 |
| (+)-Fargesin | 371.1475 | 8.4072 |
| Adenosine, 9CI, 8CI, BAN, USAN: N6- | 372.1595 | 1.7674 |
| (2-Methylbenzyl) | | |
| Tetrahydrocurcumin | 373.1658 | 0.8356 |
| 2,7-Dihydroxy-2H-1,4-benzoxazin-3(4H)- | 374.1120 | 0.0128 |
| one, 9CI; (R)-form: N-Hydroxy, O7-Me, | | |
| 2-O-β-D-glucopyranoside | | |
| Galanthamine‡, 9CI; (−)-form: O-(3R- | 374.1935 | 0.1217 |
| Hydroxybutanoyl) | | |
| Lanopylins; Lanopylin J2 | 374.3694 | 0.0425 |
| Ochropposinine oxindole | 375.2361 | 0.0814 |
| 13(24),17-Cheilanthadiene-6,19-diol | 375.3252 | 0.0518 |
| 1,5,6-Vouacapanetriol; (1a,5a,6β)-form: 6- | 377.2407 | 0.0330 |
| Ac | | |
| 3-Hydroxycholan-24-oicacid; (3β,5a)-form | 377.3028 | 0.0174 |
| 24,25-Dinor-1,3,5(10)-lupatriene | 379.3351 | 0.0608 |
| Cacospongin B: p-Quinone | 381.2755 | 0.1185 |
| 3,5,7-Tribromo-6-methoxy-1H-indole, 9CI | 381.8716 | 0.0195 |
| Antibiotic WJ 85: 5-Hydroxy, 5,10- | 382.0627 | 0.0198 |
| quinone | | |
| Plumbemycin A | 382.1105 | 0.0302 |
| Isotylocrebrine; (S)-form: 14a-Hydroxy, O3, | 382.1582 | 0.0302 |
| O6-di-de-Me | | |
| CyclomicrobuxeineK: N-De-Me,N- | 382.2812 | 0.1275 |
| formyl | | |
| Ergosta-7,22-diene | 383.3721 | 0.6936 |
| N,N-Dimethyladenosine, 9CI, 8CI: 2′,3′-O- | 384.1609 | 0.2067 |
| Benzylidene | | |
| CyclobuxophyllineO: N,N-Di-Me | 384.3314 | 0.0279 |
| 5,5′-Dibromo-2′,6′,6′- | 385.0168 | 0.0466 |
| trimethylspiro[benzofuran-2(3H),1′- | | |
| cyclohex-2′-ene] | | |
| Sesangolin | 385.1338 | 1.5699 |
| Nocardicin G | 386.1359 | 0.5545 |
| 2,3,5-Tribromo-6-(1-oxopropyl)-4H- | 386.8490 | 0.0376 |
| pyran-4-one, 9CI | | |
| 3′,6-Dichloro-4′,5,7-trihydroxyisoflavone: | 386.9555 | 0.0316 |
| 8-Chloro, 7-Me ether | | |
| Chondriol: Ac | 387.0481 | 0.0187 |
| Khellactone; (9RS,10RS)-form: 10- | 387.1417 | 0.2455 |
| Tigloyl, 9-Ac | | |
| Myriocin: 4-Deoxy, 6,7-dihydro | 388.3090 | 0.1911 |
| Lunatoic acid A | 389.1576 | 0.0139 |
| 3-Hydroxy-6-oxocholan-24-oic acid | 391.2850 | 0.0738 |
| 3,20-Diaminopregn-5-ene-16,18-diol; | 391.3319 | 0.0397 |
| (3β,16a,20S)-form: N3,N20,N20-Tri-Me | | |
| 24-Nor-4(23),9(11)-fernadiene | 395.3634 | 0.3698 |
| Inandenin-10-one | 396.3566 | 0.0287 |
| 8-Daucene-4,6,10-triol; (4β,6a,10a)-form: | 397.2552 | 0.0338 |
| 8a,9a-Epoxide, 6-(3-methylbutanoyl), 10- | | |
| Ac | | |
| Radiclonic acid | 397.3047 | 0.0411 |
| 24-Nor-12-ursene, 9CI | 397.3879 | 0.8458 |
| 3-(13-Carboxy-14,15- | 399.2658 | 0.1427 |
| dihydroxyhexadecyl)-5-methyl-2(5H)- | | |
| furanone | | |
| Buxupapine | 399.3750 | 0.0761 |
| Nocardicin E | 400.1141 | 0.0581 |
| Indicolactone: 2′,3′-Dihydro, 2′,3′- | 401.1290 | 0.0759 |
| dihydroxy | | |
| Oxopropaline D; (R)-form: 2′-Deoxy, 3′- | 401.1776 | 0.0405 |
| O-a-L-rhamnopyranoside | | |
| 3-Hydroxycholest-5-en-24-one, 9CI | 401.3342 | 0.2237 |
| Citreoviridin C | 403.2149 | 0.0422 |
| Zuelanin | 403.2584 | 0.0471 |
| 2-Methoxy-4-(2-propenyl)phenol, 9CI: | 403.3218 | 0.0972 |
| Hexadecanoyl | | |
| 13,17,19-Villanovanetriol; (ent-13β)-form: | 407.3063 | 0.0974 |
| 19-O-(3-Methylbutanoyl) | | |
| 3,18,20-Filicatriene | 407.3698 | 0.0372 |
| Dimethamine | 409.2621 | 0.0603 |
| 11,13(18)-Oleanadiene | 409.3891 | 0.1246 |
| 2-(Aminomethyl)-2-propenoic acid, 9CI: N- | 410.3691 | 0.0386 |
| Eicosanoyl, Me ester | | |
| 14-Heptacosanone: Oxime | 410.4291 | 0.0610 |
| 3,4-Dihydro-3,6,8,9-tetrahydroxy-3- | 411.2146 | 0.0342 |
| methyl-1(2H)-anthracenone; (S)-form: 6- | | |
| O-(3,7-Dimethyl-2E,6-octadienyl) | | |
| Jaspic acid | 411.2922 | 0.0713 |
| Eupha-7,24-diene; (20S)-form | 411.4014 | 0.5213 |
| Austalide K | 413.2343 | 0.0673 |
| Buxupapine: N3-Me | 413.3944 | 0.2666 |
| 15-Azasterol: 24,28-Dihydro | 414.3808 | 0.0312 |
| Crispatone | 415.2571 | 0.3074 |
| Aleicide B | 416.2772 | 0.0180 |
| Pregnane-3,5,6,8,12,14,17,20-octol | 417.2526 | 0.0173 |
| 3,25-Dihydroxy-9,10-secocholesta-5,7- | 417.3434 | 0.1109 |
| dien-24-one, 9CI | | |
| Axinellamine B‡ | 419.3411 | 0.1842 |
| Phloeodictyne A; Phloeodictyne 4,7a | 420.3698 | 0.0326 |
| Lincomycin, BAN, INN: S-Oxide | 423.2245 | 0.0374 |
| 3,7,23-Trihydroxycholan-24-oic acid; | 423.3198 | 0.0859 |
| (3a,5β,7a,23R)-form: Me ester | | |
| 23,29-Imino-B (9a)-homo-19- | 423.3643 | 0.0468 |
| norstigmasta-1(10),7,9(11),23(N)-tetraen- | | |
| 3-; (3a,5a,24?)-form: 9,11-Dihydro | | |
| 5-Octacosenoic acid | 423.4172 | 0.0591 |
| Triacontane | 423.4851 | 0.0808 |
| Plakinamine A: 24,25-Dihydro | 425.3979 | 0.2288 |
| Xestosterol | 427.3874 | 0.2549 |
| Glycerol 1-alkyl ethers; Glycerol 1- | 429.3513 | 0.0731 |
| octadecyl ether: Di-Ac | | |
| 3-Hydroxymethyl-A-norgorgostane | 429.4136 | 0.0539 |
| Lankamycin, 9CI: Aglycone, 8-deoxy, O- | 431.2957 | 0.0287 |
| de-Ac | | |
| 8,11′; 12,12′-Bi[1(10),7-eremophiladien-9- | 433.3164 | 0.2109 |
| one] | | |
| 3-Hydroxycholan-24-oicacid; (3a,5β)-form: | 434.3292 | 0.2250 |
| Glycine amide | | |
| 4,15,26-Triacontatriene-1,12,18,29- | 435.3289 | 0.1915 |
| tetrayne-3,28-diol; (3?,4E,15Z,26E,28?)- | | |
| form: 12,13-Dihydro(Z-) | | |
| Veratramine: 23-Deoxy, N—Ac | 436.3184 | 0.0815 |
| 4,15,26-Triacontatriene-1,12,18,29- | 437.3453 | 0.1380 |
| tetrayne-3,28-diol; (3S,15Z,28S)-form: | | |
| 4,5,26,27-Tetrahydro | | |
| Amphiasterin B3 | 439.3821 | 0.1762 |
| 8,13-Epoxy-14,15,16,19-labdanetetrol; (ent- | 441.3197 | 0.0234 |
| 8a,13R,14S)-form: 19-(3- | | |
| Methylbutanoyl) | | |
| Enterocin | 445.1189 | 0.0330 |
| Quinine, BAN: O-(2-Hydroxybenzoyl) | 445.2060 | 0.0196 |
| Antibiotic I1 | 445.2569 | 0.0153 |
| 2,3-Dihydroxyspirost-9(11)-en-12-one | 445.3040 | 0.0170 |
| Mutamicin 5 | 447.2934 | 0.0361 |
| Spirostane-1,3,5-triol | 449.3315 | 0.1723 |
| 1-Cyclopentyl-4-hexacosanone | 449.4764 | 0.0331 |
| Severibuxine | 450.3081 | 0.0749 |
| 3-Hydroxy-7,9(11),22,24-lanostatetraen- | 451.3269 | 0.4921 |
| 26,23-olide | | |
| 4-Methylaconitane-1,6,7,8,14,16,18-heptol; | 452.2552 | 0.0310 |
| (1a,5β,6β,14a,16β)-form: 14-Ketone, O6,O16, | | |
| O18-tri-Me, N-Et | | |
| 4-Methylaconitane-6,7,8,14,16,18-hexol; | 452.3056 | 0.2497 |
| (5β,6β,14a,16β)-form: O6,O14,O16,O18- | | |
| Tetra-Me, N-Et | | |
| 3,5-Dioxooctacosanoicacid | 453.3996 | 0.2882 |
| Spirosol-4-en-3-one, 9CI; (22R,25R)- | 454.3393 | 0.1406 |
| form: N—Ac | | |
| 7-[5-(Decahydro-4a-hydroxy-1,2,5,5- | 455.3303 | 0.0595 |
| tetramethyl-1-naphthalenyl)-3-methyl-2- | | |
| N-Deformyldichotamine: 10,11- | 457.2315 | 0.1032 |
| Dimethoxy, N-propanoyl | | |
| Abyssinine B | 459.2943 | 0.1964 |
| Anopteryl alcohol: 12-Tigloyl | 460.2701 | 0.1310 |
| 3′,4′,5,7-Tetrahydroxyflavone: 3′-O-β-D- | 463.0925 | 0.0304 |
| Glucuronopyranoside | | |
| 3-Deoxy-manno-oct-2-ulosonic acid, 9CI; | 463.1551 | 0.0568 |
| D-Furanose-form: 2,4,6,7,8-Penta-Ac, Me | | |
| ester | | |
| Urceolide | 463.2197 | 0.0346 |
| Spiropachysine | 463.3646 | 0.1943 |
| Lincosamine; a-Pyranose-form: 1-Thio, Me | 464.1552 | 0.1061 |
| glycoside, penta-Ac | | |
| 3,5-Acarnidine: 5,6Z-Didehydro | 464.4043 | 0.1023 |
| 2,3,14,20,25-Pentahydroxycholest-7-en-6- | 465.3246 | 0.1089 |
| one | | |
| 10,12-Hentriacontanedione | 465.4705 | 0.2256 |
| Indanomycin: 16-Deethyl | 466.3003 | 0.0317 |
| Teleocidin B1: 16-Epimer, Me ether | 466.3436 | 0.0236 |
| Cephaeline; (−)-form | 467.2937 | 0.3117 |
| Trideacetylpyripyropene A: 11-Epimer, | 468.2441 | 0.0502 |
| 7,19-dideoxy, 3-Ac | | |
| Cytosine arabinoside; β-D-Furanose-form: | 468.3372 | 0.0732 |
| N4-Hexadecyl | | |
| 3,29-Dihydroxy-12-oleanen-27-oic acid; | 469.3380 | 0.0869 |
| 3a-form: 3-Ketone, 29-aldehyde | | |
| Tryptoquivaline N | 473.1871 | 0.0542 |
| Botcineric acid: 3-Ac | 473.2676 | 0.0333 |
| Mucronine C | 473.3172 | 0.0353 |
| 3,29-Dihydroxy-12-oleanen-27-oic acid | 473.3658 | 0.0405 |
| 2,29-Diamino-5,8,11,14,17,20- | 473.4145 | 0.0841 |
| triacontahexaene-3,28-diol, 9CI | | |
| Lanost-9(11)-ene-3,24,25-triol; (3β,5a,24S)- | 475.4150 | 0.0365 |
| form: 3-Me ether | | |
| Russuphelin C: 1-Me ether | 476.9480 | 0.0201 |
| Austalide H | 477.2405 | 0.0112 |
| Desoxophylloerythroetioporphyrin | 477.3036 | 0.0167 |
| Griseoviridin | 478.1682 | 0.0073 |
| 8-Dotriacontenoicacid | 479.4900 | 0.2775 |
| 2,4,6,8-Tetramethyloctacosanoic acid | 481.4898 | 0.0822 |
| Stawamycin | 482.2926 | 0.0716 |
| 2,3-Dihydroxy-24-nor-6-oxo-1,3,5(10)- | 483.3136 | 0.0536 |
| friedelatrien-29-oicacid: Me ester | | |
| Cycloheterophyllin: 2-Deoxy | 487.2192 | 0.1422 |
| Antibiotic A 2315C | 488.2376 | 0.0933 |
| Budmunchiamine L5‡ | 493.4922 | 0.3113 |
| Misenine | 495.4278 | 0.3000 |
| Lipstatin: Tetrahydro | 496.4059 | 0.0290 |
| CyclovirobuxeineI: N3,N3,N20-Tri- | 497.4057 | 0.0452 |
| Me,O-tigloyl | | |
| Tetradecanoic acid, 9CI: 2,3- | 497.4552 | 0.0308 |
| Dihydroxyheptadecenyl ester | | |
| Pseudomonic acid A: 4′,5′-Didehydro | 499.2922 | 0.0194 |
| Nemorosone | 503.3065 | 0.1051 |
| Cycloprotobuxine I: 6,7-Didehydro, N3,N20, | 503.3904 | 0.0314 |
| N20-tri-Me,N3-benzoyl | | |
| 7-Oxotetratriacontanal | 507.5223 | 0.2955 |
| 31-Methyltritriacontanoic acid | 509.5391 | 0.0495 |
| 3-Methyl-3-buten-1-ol: Triacontanoyl | 521.5231 | 0.2833 |
| Tetrahydro-2-(1-hydroxy-9-nonenyl)-5- | 523.4738 | 0.0939 |
| pentyl-3-furanol: 1′-O-Tetradecanoyl | | |
| Murrafoline C | 527.2702 | 0.0541 |
| 2,3,7,11,15-Pentahydroxy-18- | 527.3560 | 0.0166 |
| hydroxymethyl-2,6,10,14,16,20- | | |
| hexamethyl-4,8,12,16-docosatetraenoic | | |
| acid, 9CI | | |
| 9-Octadecenyl 9-octadecenoate, 9CI | 533.5258 | 0.1227 |
| Artemoin A | 551.5074 | 0.0938 |
| 3′-(8,17-Epoxy-16-oxo-12,14-labdadien- | 571.3102 | 0.1142 |
| 15-yl)-2′,4′-dihydroxy-6′- | | |
| methoxychalcone | | |
| Montecristin | 575.5089 | 0.0438 |
| Bombiprenone | 603.5416 | 0.1532 |
| 1,8,9,14-Tetrahydroxydihydro-β- | 608.2954 | 0.0628 |
| agarofuran; (1a,8β,9a)-form: 14-(3- | | |
| Pyridinecarbonyl), 9-benzoyl, 1-(2- | | |
| methylpropanoyl), 8-Ac | | |
| Jolantinine | 609.3402 | 0.1489 |
| Haliclotriol A | 609.4168 | 0.0416 |
| Dimethylmenaquinone | 609.4754 | 0.0388 |
| Maytansinol: 3-O-(3-Hydroxy-3- | 651.2745 | 0.0364 |
| methylbutanoyl), N-de-Me | | |
| Quercetin 3-glycosides; Monosaccharides: | 757.1737 | 0.0352 |
| 3-O-[3,6-Bis(4-hydroxy-E-cinnamoyl)-β- | | |
| D-glucopyranoside] | | |
| 2,3,5,7,8,9,15-Heptahydroxy-6(17),11- | 757.3037 | 0.0265 |
| jatrophadien-14-one; | | |
| (2a,3β,5a,7β,8a,9a,11E,15β)-form: 7- | | |
| Benzoyl, 2,3,5,8,9,15-hexa-Ac | | |
| Hydroxystreptomycin B | 760.3216 | 0.0985 |
| Acylsucroses: 2,3′,4′,6′-Tetrakis(3- | 763.4199 | 0.0883 |
| methylbutanoyl), 1′-(2S-methylbutanoyl) | | |
| Pregn-5-ene-3,14,20-triol; (3β,14β,20R)- | 819.4401 | 0.0419 |
| form: 3-O-[β-D-Glucopyranosyl-(1?4)-β- | | |
| D-digitalopyranoside], 20-O-β-D- | | |
| glucopyranoside | | |
| Sulfurmycin B: 1-Hydroxy | 854.3137 | 0.0426 |
| Itampolin A | 859.9955 | 0.0149 |
| 3-Phosphatidylinositol; Glycerol 1-(9,12- | 861.5496 | 0.0104 |
| octadecadienoate) 2-(9-octadecenoate) 3- | | |
| phosphoinositol | | |
| Antibiotic 1176A | 862.4856 | 0.0112 |
| Glycerol trialkanoates (diacid, | 887.7979 | 0.0084 |
| unsymmetrical); Glycerol 1,2- | | |
| dioctadecanoate 3-(9Z,12Z- | | |
| octadecadienoate) | | |
| Lyngbyabellin D | 896.2684 | 0.0167 |
| Huratoxin: 5-Deoxy, 6,7-deepoxy, 6,7- | 903.7036 | 0.0382 |
| didehydro, 20-tetracosanoyl | | |
| Quercetin 3,7-diglycosides: 3-O-[3,4- | 905.2383 | 0.0345 |
| Dihydroxy-E-cinnamoyl-(?4)-a-L- | | |
| rhamnopyranosyl-(1?2)-a-L- | | |
| arabinopyranoside], 7-O-β-D- | | |
| glucopyranoside | | |
| Periplaneta americana Pyrokinins; Pea-PK-3 | 996.6478 | 0.1808 |

TABLE 4
Chemicals present in Extract 2 as determined by DART
TOF-MS analysis and utilization of a searchable database.

| | | Relative |
|---|---|---|
| | Measured | Abundance |
| Compound Name | Mass | (%) |
| 3-Methyl-2-butenoic acid, 9CI | 101.0537 | 0.4031 |
| 4-Fluorobutanoic acid, 9CI | 107.0489 | 0.4982 |
| 1,2-Dimethylbenzene, 9CI | 107.0861 | 0.7179 |
| 1,3,6-Octatriene | 109.1005 | 1.2655 |
| 2-Methyl-1-cyclopentene-1- | 111.0791 | 0.4599 |
| carboxaldehyde | | |
| 3-Aminobutanoic acid, 9CI; (±)-form: N,N- | 113.1093 | 0.3598 |
| Di-Me, nitrile | | |
| 1-Ethyl-2-methylcyclopentane, 9CI | 113.1323 | 0.3091 |
| 2-Methylaminoacetic acid: Et ester | 118.0838 | 0.2594 |
| 2,3-Diaminopropanoicacid; (R)-form: N3- | 119.0857 | 19.0811 |
| Me | | |
| Isopropylbenzene, 8CI | 121.1004 | 5.7696 |
| 5-Ethyl-2-methylpyridine | 122.1031 | 0.8927 |
| Benzoic acid, 9CI, USAN | 123.0485 | 2.6511 |
| (2-Hydroxyethyl)dimethyl- | 124.0499 | 0.6857 |
| sulfoxonium(1+) | | |
| 2-Chloro-2-propenoic acid, 9CI: Chloride | 124.9514 | 0.2165 |
| 6-Methyl-3,5-heptadien-2-one, 9CI; (E)- | 125.0980 | 2.2472 |
| (?)-form | | |
| 2-Amino-4-hydroxypyrimidine; 1H-form: | 126.0739 | 0.6397 |
| 1-Me | | |
| 1-Isothiocyanato-2-methylbutane | 130.0718 | 0.8910 |
| 3-Methyl-1-butylamine: N-Propyl | 130.1601 | 0.8411 |
| 2-Methyl-3,4-piperidinediol, 9CI | 132.1030 | 1.3284 |
| 2,4-Diaminopentanoicacid | 133.1023 | 3.7432 |
| Noractinidine | 134.1049 | 0.2179 |
| Glycerol, INN: Tri-Me ether | 135.1000 | 7.5142 |
| 2-Acetylpyridine: Hydrazone | 136.0936 | 1.4116 |
| 4-Methylbenzoic acid, 9CI | 137.0644 | 3.9742 |
| 2-Hydroxybenzoic acid, 9CI | 139.0490 | 0.8541 |
| 2-Pentylfuran | 139.1137 | 0.6348 |
| 2-Ethyl-2,3-dihydro-6-methyl-4H-pyran- | 141.0952 | 0.4202 |
| 4-one, 9CI | | |
| 2-Butanol, 9CI; (±)-form: 2-Methyl-2- | 143.1047 | 0.3330 |
| propenoyl | | |
| 2-Amino-3-hydroxymethyl-3-pentenoic | 146.0887 | 0.6813 |
| acid, 8CI | | |
| 2,5-Furandiacetic acid, 9CI: Dinitrile | 147.0475 | 2.3155 |
| 2-Hydroxybenzoic acid, 9CI: Et ether, | 148.0664 | 0.3968 |
| nitrile | | |
| 2-Amino-5-hydroxyhexanoic acid | 148.0944 | 0.2502 |
| Diethanolamine: N-Propyl | 148.1256 | 0.4270 |
| 3-(2-Pyrrolidinyl)pyridine, 9CI | 149.1147 | 2.4502 |
| 4-Methylbenzoic acid, 9CI: Methylamide | 150.0973 | 0.7038 |
| 2,6-Decadien-4-yn-1-ol, 8CI | 151.1145 | 6.9246 |
| decadienal | 152.0596 | 0.4260 |
| 2-Vinyl-1,3,5-benzenetriol | 153.0564 | 10.2125 |
| Cytosine: 4-N—Ac | 154.0617 | 0.7732 |
| 4-Hydroxy-2-cyclopenten-1-one, 9CI; (R)- | 155.1002 | 0.5304 |
| form: tert-Butyl ether | | |
| 5-Methyl-2,3-hexanedione, 9CI: Dioxime | 159.1179 | 1.4088 |
| Putreanine | 161.1321 | 1.8691 |
| Boschniakine; (R)-form | 162.0950 | 0.3639 |
| Safrole | 163.0784 | 4.8994 |
| 2-Amino-4,5-dihydroxy-4-methylpentanoic | 164.0871 | 1.2808 |
| acid | | |
| Benzylamine, 8CI: N,N-Di-Et | 164.1409 | 0.7453 |
| Eugenol | 165.0823 | 7.7394 |
| 2-Amino-2-phenylpropanoic acid | 166.0793 | 1.6718 |
| 3-Hydroxy-p-mentha-1,8-dien-7-al | 167.1077 | 3.0020 |
| 10-Hydroxy-4-pinanone | 169.1223 | 3.5416 |
| 2-Aminoethanol, 9CI: N,N-Di-Et, 2- | 172.1375 | 0.6247 |
| propenoyl | | |
| 1,4-Diguanidinobutane | 173.1421 | 1.5657 |
| 2-Amino-4-ethylidenepentanedioic acid | 174.0694 | 0.6733 |
| Indospicine | 174.1269 | 0.6411 |
| 1,4-Diamino-1,4-dideoxyglucitol, 9CI; Di- | 174.2019 | 0.2183 |
| NH-form: N1,N3,N3′-Tri-Me | | |
| N-Methyltryptamine | 175.1234 | 3.0167 |
| 4-Amino-1-phenyl-1-penten-3-one, 9CI | 176.1139 | 1.0889 |
| 2-Amino-3-(oxalylamino)propanoic acid | 177.0572 | 30.5639 |
| Cycloalliin | 178.0614 | 4.6637 |
| Nicotine, BSI, ISO; (S)-form: 1′-N-Oxide | 179.1280 | 6.6233 |
| Tecomanine | 180.1371 | 1.6353 |
| Herbipoline | 181.1023 | 1.5529 |
| 1-Methyl-9H-carbazole, 9CI | 182.1052 | 0.5233 |
| Dendrobates Alkaloid 181A | 182.1980 | 0.2337 |
| Dihydro-5-(5-hydroxy-1,3-hexadienyl)-2(3H)- | 183.0987 | 1.5754 |
| furanone | | |
| 6-Tridecene | 183.2071 | 0.3163 |
| 3,7-Dimethyl-1-(methylthio)-2,6- | 185.1294 | 0.6142 |
| octadiene; (E)-form | | |
| Dihydro-5-(4-hydroxy-4-methylpentyl)-2(3H)- | 187.1388 | 0.9120 |
| furanone | | |
| 1H-Indole-3-methanamine: 3-N—Ac | 189.1066 | 2.3828 |
| 1-Isothiocyanato-6-(methylthio)hexane, | 190.0660 | 3.5975 |
| 9CI | | |
| Rhizobitoxin | 191.1075 | 3.2206 |
| 2-Amino-2-deoxyribose; D-form: N—Ac | 192.0829 | 0.5215 |
| 5-(1-Hydroxyethyl)-7-methoxybenzofuran | 193.0950 | 9.5343 |
| N-Benzoylglycine, 9CI: Hydrazide | 194.0919 | 2.6954 |
| 3,4-Dihydro-3,8-dihydroxy-3-methyl-1H- | 195.0741 | 12.8862 |
| 2-benzopyran-1-one, 9CI | | |
| Asterubin | 196.0822 | 1.2064 |
| 4-(3-Aminopropyl)-1,2-benzenediol, 9CI: | 196.1414 | 0.4040 |
| Di-Me ether | | |
| Elaeokanine A; (S)-form: 1′-Alcohol (1′S- | 196.1766 | 0.3322 |
| ?) | | |
| 2-Acetyl-4,4,6-trimethyl-1,3- | 197.1148 | 1.4141 |
| cyclohexanedione, 9CI | | |
| 2,8,10-Pentadecatriene-4,6-diyne, 8CI | 199.1424 | 2.5627 |
| Tetradecane | 199.2390 | 0.3881 |
| a-Amino-4-carboxy-3-furanpropanoic acid, | 200.0568 | 0.0772 |
| 9CI; (S)-form | | |
| 2-Pentylquinoline, 9CI | 200.1531 | 0.1562 |
| Dodecanoicacid, 9CI: Amide | 200.1928 | 0.1075 |
| 1,3,5,7-Cadinatetraene, 8CI | 201.1663 | 3.9367 |
| 2-Aminoheptanoic acid; (±)-form: N-Di- | 202.1745 | 0.7294 |
| Me, Et ester | | |
| 3,10(14)-Aromadendradiene | 203.1810 | 8.5977 |
| 3,5-Cadinadiene | 205.1994 | 12.8178 |
| Dendrobates Alkaloid 203: Dihydro (?) | 206.1992 | 1.2783 |
| 6-Deoxyglucose, 9CI, 8CI; β-L-Pyranose- | 207.1253 | 2.7385 |
| form: 3-Me, Et glycoside | | |
| Felinine | 208.0934 | 1.1615 |
| Xanthostemone | 209.1192 | 1.5941 |
| Triacsin B: 6,7,8,9-Tetrahydro | 210.1633 | 0.7940 |
| Antibiotic A 41-89; Antibiotic A 41-89I | 211.1352 | 1.2137 |
| Linderazulene: 2,3-Dihydro | 213.1368 | 1.2705 |
| Hexahydro-7a-hydroxy-3H-pyrrolizin-3- | 214.1389 | 0.6770 |
| one, 9CI; (±)-form: (1-Ethoxyethyl) ether | | |
| Tsitsikammafuran | 215.1453 | 4.3919 |
| Decanedioic acid, 9CI: Amide-Me ester | 216.1532 | 2.1994 |
| Verboccidentafuran | 217.1606 | 100.0000 |
| N-Deacetylkuanoniamine D; (Z)-form: 2- | 218.1635 | 16.8742 |
| Methylpropylamide | | |
| 1,3,5-Cadinatrien-10-ol; (7a H,10a)-form | 219.1764 | 65.9700 |
| Sedamine | 220.1796 | 10.0779 |
| 1(10),4(15)-Lepidozadien-5-ol | 221.1948 | 6.1817 |
| 2-Hydroxybenzoic acid, 9CI: | 223.1056 | 0.7866 |
| Tetrahydrofurfuryl ester | | |
| Murexine | 225.1460 | 1.1425 |
| 5,10-Pentadecadien-1-ol | 225.2225 | 0.3922 |
| 1-Hexadecene | 225.2582 | 0.4977 |
| 2,4-Dialkylthiazoles; 4-Ethyl-2- | 226.1645 | 0.1920 |
| octylthiazole, 9CI | | |
| 9-Tetradecenoic acid; (E)-form | 227.2043 | 0.4303 |
| Faramol | 229.1272 | 0.7961 |
| Tetradecanoic acid, 9CI | 229.2186 | 0.6846 |
| Verboccidentafuran: 2-Oxo | 231.1412 | 4.4343 |
| 2-Aminoheptanedioic acid, 8CI; (±)-form: | 232.1502 | 1.6351 |
| Di-Et ester | | |
| Verboccidentafuran: 4a,5a-Epoxide | 233.1546 | 28.2276 |
| Hypusine | 234.1718 | 6.7467 |
| 9-Hydroxy-4,10(14)-oplopadien-3-one | 235.1709 | 29.3531 |
| 3,10(14),11-Germacratriene-1,9-diol | 237.1914 | 5.4544 |
| Dendrobates Alkaloid 237E | 238.2078 | 1.0228 |
| 1,2,3,4-Tetrahydro-5,6,7- | 239.1521 | 0.4634 |
| trihydroxyisoquinoline: 6,7-Di-Me ether, | | |
| N,N-di-Me | | |
| 10-Propyl-5,9-tridecadien-1-ol, 9CI; (Z)- | 239.2365 | 1.9840 |
| form | | |
| Spherophysine: N4′-Ac | 241.2064 | 0.7295 |
| Pearlmycin, 9CI | 241.2546 | 0.4327 |
| Arabinitol, 9CI; D-form: 1-Benzyl | 243.1260 | 0.7702 |
| 2,6,10-Trimethyldodecanoic acid | 243.2351 | 1.4679 |
| Vitamin H (biotin) | 245.0848 | 24.2464 |
| Mycosporin-Gly | 246.0904 | 3.3109 |
| 8-Hydroxy-1,4,7(11)-guaiatrien-12,8-olide | 247.1390 | 3.9514 |
| 5-(2-Hydroxyethyl)-4-methylthiazole: | 248.0741 | 0.2599 |
| Benzoyl | | |
| 4-Amino-4,6-dideoxy-3-C- | 248.1430 | 1.2133 |
| methylmannose; β-D-Pyranose-form: Me | | |
| glycoside, N-Me, N—Ac | | |
| 6-Hydroxy-4,11(13)-eudesmadien-12,8- | 249.1534 | 7.9083 |
| olide | | |
| 4-Amino-4-deoxyglucuronic acid; a-D- | 250.0899 | 0.1357 |
| Pyranose-form: Me glycoside, N—Ac | | |
| Fungerin: N1-Me | 250.1701 | 1.8025 |
| Thienodolin | 250.9980 | 0.0907 |
| 1-(3,5-Dichloro-4-methoxyphenyl)-1,2- | 251.0336 | 0.1062 |
| propanediol, 9CI | | |
| 1-Hydroxy-5,11(13)-eudesmadien-12-oic | 251.1679 | 13.1073 |
| acid | | |
| 2′-Deoxyadenosine, 9CI, 8CI | 252.1104 | 0.0875 |
| Arglecin | 252.1836 | 2.7169 |
| 1-Hydroxy-4(15),11(13)-eudesmadien-12- | 253.1809 | 2.9117 |
| oic acid; (1a,7β)-form: 11,13-Dihydro | | |
| 2-Amino-11,15-hexadecadien-3-ol | 254.2446 | 1.5639 |
| Echinaxanthol | 255.2309 | 6.5553 |
| Dendrobates Alkaloid 253B | 256.2584 | 1.3474 |
| 8-Methylpentadecanoic acid, 9CI | 257.2538 | 7.7557 |
| 2-(2-Hydroxybutyl)-6-(2-hydroxypentyl)- | 258.2490 | 1.1524 |
| 1-methylpiperidine | | |
| Lyconadine A: 2,3-Dihydro | 259.1898 | 3.1360 |
| Obliquin; (S)-form: 5′-Hydroxy | 261.0792 | 0.4561 |
| 1,3,6,9-Nonadecatetraene | 261.2495 | 0.6129 |
| 8-Methyl-8-azabicyclo[3.2.1]octane-3,6- | 262.1411 | 0.6029 |
| diol, 9CI; (3R,6R)-form: 3-Benzoyl | | |
| 10-Hydroxy-1,3,5-cadinatrien-15-oic acid; | 263.1569 | 2.4585 |
| (7β,10a OH)-form: Me ester | | |
| 3-(Dimethylaminomethyl)-5- | 265.1549 | 5.5185 |
| hydroxyindole: ar, N-Dimethoxy, Me | | |
| ether | | |
| Brevicolline; (S)-form | 266.1627 | 1.5393 |
| 3,8-Dihydroxy-4(15),9,11(13)- | 267.1629 | 2.5805 |
| germacratrien-12,6-olide; (3β,6a,8a,9E)- | | |
| form: 11a,13-Dihydro | | |
| 9-Octadecenal | 267.2661 | 1.0459 |
| Brevicarine | 268.1866 | 0.4934 |
| 14-Pentadecenoic acid: Et ester | 269.2439 | 3.3754 |
| 3-Methylpentadecanoic acid, 9CI; (±)- | 271.2566 | 1.0647 |
| form: Me ester | | |
| 2,4-Diamino-5,6-dihydroxypyrimidine: 5- | 275.1025 | 0.2939 |
| O-Arabinopyranoside | | |
| 1,7,16-Hexadecanetriol; (?)-form | 275.2594 | 1.1894 |
| 8-Methyl-8-azabicyclo[3.2.1]octane-3,6- | 276.1563 | 0.2917 |
| diol, 9CI; (3R,6R)-form: 3-O- | | |
| Phenylacetyl | | |
| 3,18-Heneicosadiene-1,8,10,20-tetrayne; | 277.1995 | 0.6758 |
| (Z,Z)-form | | |
| 7-Hydroxy-14,15-dinor-8(17)-labden-13- | 279.2282 | 1.7996 |
| one | | |
| 2,4-Tetradecadienoic acid, 9CI; (2E,4E)- | 280.2618 | 1.9791 |
| form: 2-Methylpropylamide | | |
| 1,13-Dihydroxy-4-oxo-2-pseudoguaien- | 281.1488 | 0.8665 |
| 12,6-olide | | |
| 9,12-Octadecadienoic acid, 9CI; (9E,12Z)- | 281.2570 | 3.2199 |
| form | | |
| 10-Octadecenoic acid; (E)-form: Amide | 282.2817 | 11.1107 |
| 5-(10- | 283.2752 | 8.0477 |
| Aminoundecyl)hexahydropyrrolo[2,1-b]oxazole | | |
| Heptadecanoic acid: Me ester | 285.2822 | 4.0818 |
| 1-Piperoylpiperidine | 286.1542 | 1.8801 |
| 2-Amino-15-methyl-4-hexadecene-1,3-diol | 286.2766 | 0.7064 |
| 2-Amino-3-octadecanol | 286.3173 | 0.2569 |
| Komaroine | 287.1530 | 0.1845 |
| N-(3-Hydroxy-1-oxocyclopent-2-en-2-yl)- | 290.0959 | 0.3904 |
| 3-(4-hydroxy-3-methoxyphenyl) | | |
| 3-Docosene-1,11,13,15,21-pentayne,9CI; | 291.2184 | 0.5786 |
| (Z)-form: Dihydro | | |
| Lasiodiplodin; (R)-form: 6-Oxo, O-de-Me | 293.1465 | 3.1262 |
| 10(14)-Aromadendrene-2,3,4-triol; | 295.1952 | 2.4985 |
| (1a,2a,3a,4a,5a,6β,7β)-form: 2-Ac | | |
| 2-Amino-4,8,10-octadecatriene-1,3-diol | 296.2630 | 0.3629 |
| Dendrobates Alkaloid 295 | 296.3014 | 0.1669 |
| 3-O-Methylgalactose, 9CI, 8CI; a-D- | 297.1303 | 4.7602 |
| Pyranose-form: Me glycoside, 4,6-O- | | |
| benzylidene | | |
| Cassine; (−)-form | 298.2832 | 0.5762 |
| 1,10:4,5-Diepoxy-3,6,8-trihydroxy-11- | 299.1524 | 0.5734 |
| germacren-9-one; (1β,3a,4a,5a,6a,8a,10β)- | | |
| form | | |
| 4-Oxooctadecanoic acid | 299.2580 | 0.4199 |
| Palmidrol, INN | 300.2861 | 0.3561 |
| 2′,3′,4,4′,6,7-Hexahydroxyisoflavan | 307.0906 | 0.2883 |
| 2-Hydroxydodecanoic acid; (R)-form: | 307.2310 | 1.2488 |
| Benzyl ester | | |
| Aspergillomarasmine A | 308.1099 | 0.8336 |
| Bisdemethoxycurcumin | 309.1141 | 4.5400 |
| 1H-Indole-5,6-diol, 9CI: N,O6-Disulfo | 309.9685 | 0.0919 |
| Mescaline succinimide: 3-Hydroxy | 310.1218 | 6.7771 |
| 1,1,3-Tribromo-3-chloro-1-propene | 310.8100 | 0.1144 |
| Antibiotic A11-99-1 | 310.8531 | 0.1253 |
| Eupomatenoid 11 | 311.1358 | 14.3339 |
| Diiodoaceticacid: Amide | 311.8442 | 0.1975 |
| 4,5-Dibromo-1H-pyrrole-2,3-dicarboxylic | 311.8980 | 0.1032 |
| acid | | |
| 2-Amino-2-deoxygalactose, 9CI, 8CI; a-D- | 312.1430 | 2.3324 |
| Pyranose-form: Benzyl glycoside, N—Ac | | |
| 5-[(4-Hydroxyphenyl)ethenyl]-2-(3- | 313.1501 | 3.2285 |
| methyl-1-butenyl)-1,3-benzenediol: 3′- | | |
| Hydroxy | | |
| Dehydroisolongistrobine: Dihydro | 314.1527 | 0.6764 |
| Acetylleucylargininal; L-DL-form | 314.2125 | 0.1968 |
| Prosopine‡: 11′-Ketone | 314.2772 | 0.5374 |
| Flourensianol: Tigloyl | 315.1643 | 0.3532 |
| 12,13-Dihydroxy-9-octadecenoicacid | 315.2508 | 0.8270 |
| 18-Hydroxy-19-trachylobanoic acid | 319.2216 | 0.4192 |
| 2,7,11-Cembratriene-4,10-diol; (1S,2E,4R, | 319.2585 | 0.5653 |
| 7E,10R,11Z)-form: 10-Ketone, 4-Me | | |
| ether | | |
| Rutaecarpine: 7β,8a-Dihydroxy | 320.1124 | 3.6959 |
| 13-Docosenoic acid; (E)-form: Nitrile | 320.3311 | 0.5850 |
| Methyl β-D-glucopyranoside: 2,3,4-Tri-Ac | 321.1097 | 0.5047 |
| Bavachromene | 323.1324 | 0.5832 |
| Cneorumchromene G | 325.1467 | 2.3162 |
| Tetrahydrothalifendine | 326.1484 | 0.2632 |
| Galeon | 327.1505 | 6.2202 |
| Cryptostyline I; (R)-form | 328.1552 | 1.5091 |
| 9,10-Dihydroxyoctadecanoicacid; (9R,10R)- | 331.2853 | 0.6504 |
| form: Me ester | | |
| 3-(12-Phenyl-8-dodecenyl)phenol | 337.2599 | 0.7226 |
| 1H-Indol-3-ylacetyl-myo-inositol | 338.1198 | 7.2796 |
| Demethoxycurcumin | 339.1242 | 5.4562 |
| Isocorypalmine | 342.1648 | 2.8777 |
| Sorbistin C | 343.1765 | 2.1722 |
| (2,4-Dimethoxy-3- | 344.2291 | 0.8347 |
| prenylcinnamoyl)piperidine | | |
| 5,6-Dibromotryptamine: Nb,Nb-Di-Me | 344.9922 | 0.2921 |
| Chondriol | 345.0506 | 0.2122 |
| Gelsedine, 9CI: 14R-Hydroxy | 345.1830 | 0.3346 |
| Mescaline isocitrimide lactone | 350.1162 | 15.9933 |
| Estra-1,3,5(10)-triene-3,17-diol; 17β-form: | 351.1235 | 3.9473 |
| 1-Bromo | | |
| 2-Amino-3-(3,5-dibromo-4- | 351.9602 | 0.2600 |
| hydroxyphenyl)propanoic acid; (S)-form: | | |
| 4-Me ether | | |
| Eritadenine; (2R,3R)-form: 2,3-Di-Ac, | 352.1332 | 2.8102 |
| Me ester | | |
| Estra-1,3,5(10)-triene-3,17-diol; 17β-form: | 353.1457 | 3.6377 |
| 3-O-Sulfate | | |
| Hackelidine: 7-Ac | 354.1486 | 0.7516 |
| Isostemonidine | 354.2219 | 0.2850 |
| 1,3,9-Trihydroxy-10-prenylpterocarpan: 1- | 355.1530 | 9.9986 |
| Me ether | | |
| Rutacridone: 1′,2′-Dihydro, 1′-hydroxy, | 356.1494 | 2.8970 |
| 2′-methoxy | | |
| Xanthoascin | 357.1595 | 1.2042 |
| Glycerol 1-alkanoates; Glycerol 1-(9Z- | 357.3081 | 1.1649 |
| octadecenoate) | | |
| 3,14,17,21-Tetrahydroxypregn-5-en-20- | 365.2341 | 0.3998 |
| one; (3β,14β,17βOH)-form | | |
| Collinusin | 367.1181 | 3.6075 |
| Karnamicin A1: 1″-Deoxy | 368.1244 | 27.2862 |
| Curcumin | 369.1332 | 43.9843 |
| Carinatol: 7-Ketone, 9-hydroxy | 373.1658 | 5.5137 |
| 2,7-Dihydroxy-2H-1,4-benzoxazin-3(4H)- | 374.1120 | 0.0844 |
| one, 9CI; (R)-form: N-Hydroxy, O7-Me, | | |
| 2-O-β-D-glucopyranoside | | |
| Galanthamine‡, 9CI; (−)-form: O-(3R- | 374.1935 | 0.8033 |
| Hydroxybutanoyl) | | |
| Lanopylins; Lanopylin J2 | 374.3694 | 0.2803 |
| Ochropposinine oxindole | 375.2361 | 0.5368 |
| 13(24),17-Cheilanthadiene-6,19-diol | 375.3252 | 0.3419 |
| 1,5,6-Vouacapanetriol; (1a,5a,6β)-form: 6- | 377.2407 | 0.2176 |
| Ac | | |
| 3-Hydroxycholan-24-oicacid; (3β,5a)-form | 377.3028 | 0.1150 |
| 24,25-Dinor-1,3,5(10)-lupatriene | 379.3351 | 0.4014 |
| Cacospongin B: p-Quinone | 381.2755 | 0.7820 |
| 3,5,7-Tribromo-6-methoxy-1H-indole, 9CI | 381.8716 | 0.1290 |
| Antibiotic WJ 85: 5-Hydroxy, 5,10- | 382.0627 | 0.1306 |
| quinone | | |
| Plumbemycin A | 382.1105 | 0.1992 |
| Isotylocrebrine; (S)-form: 14a-Hydroxy, O3, | 382.1582 | 0.1992 |
| O6-di-de-Me | | |
| CyclomicrobuxeineK: N-De-Me,N- | 382.2812 | 0.8414 |
| formyl | | |
| Ergosta-7,22-diene | 383.3721 | 4.5769 |
| N,N-Dimethyladenosine,9CI, 8CI: 2′,3′-O- | 384.1609 | 1.3638 |
| Benzylidene | | |
| CyclobuxophyllineO: N,N-Di-Me | 384.3314 | 0.1840 |
| 5,5′-Dibromo-2′,6′,6′- | 385.0168 | 0.3076 |
| trimethylspiro[benzofuran-2(3H),1′- | | |
| cyclohex-2′-ene] | | |
| Sesangolin | 385.1338 | 10.3594 |
| Nocardicin G | 386.1359 | 3.6587 |
| 2,3,5-Tribromo-6-(1-oxopropyl)-4H- | 386.8490 | 0.2481 |
| pyran-4-one, 9CI | | |
| 3′,6-Dichloro-4′,5,7-trihydroxyisoflavone: | 386.9555 | 0.2088 |
| 8-Chloro, 7-Me ether | | |
| Chondriol: Ac | 387.0481 | 0.1231 |
| Khellactone; (9RS,10RS)-form: 10- | 387.1417 | 1.6202 |
| Tigloyl, 9-Ac | | |
| Myriocin: 4-Deoxy, 6,7-dihydro | 388.3090 | 1.2611 |
| Lunatoic acid A | 389.1576 | 0.0917 |
| 3-Hydroxy-6-oxocholan-24-oic acid | 391.2850 | 0.4868 |
| 3,20-Diaminopregn-5-ene-16,18-diol; | 391.3319 | 0.2616 |
| (3β,16a,20S)-form: N3,N20,N20-Tri-Me | | |
| 24-Nor-4(23),9(11)-fernadiene | 395.3634 | 2.4400 |
| Inandenin-10-one | 396.3566 | 0.1896 |
| 8-Daucene-4,6,10-triol; (4β,6a,10a)-form: | 397.2552 | 0.2232 |
| 8a,9a-Epoxide, 6-(3-methylbutanoyl), 10- | | |
| Ac | | |
| Radiclonic acid | 397.3047 | 0.2713 |
| 24-Nor-12-ursene, 9CI | 397.3879 | 5.5809 |
| 3-(13-Carboxy-14,15- | 399.2658 | 0.9419 |
| dihydroxyhexadecyl)-5-methyl-2(5H)- | | |
| furanone | | |
| Buxupapine | 399.3750 | 0.5019 |
| Nocardicin E | 400.1141 | 0.3837 |
| Indicolactone: 2′,3′-Dihydro, 2′,3′- | 401.1290 | 0.5010 |
| dihydroxy | | |
| Oxopropaline D; (R)-form: 2′-Deoxy, 3′- | 401.1776 | 0.2673 |
| O-a-L-rhamnopyranoside | | |
| 3-Hydroxycholest-5-en-24-one, 9CI | 401.3342 | 1.4759 |
| Citreoviridin C | 403.2149 | 0.2785 |
| Zuelanin | 403.2584 | 0.3106 |
| 2-Methoxy-4-(2-propenyl)phenol, 9CI: | 403.3218 | 0.6417 |
| Hexadecanoyl | | |
| 13,17,19-Villanovanetriol; (ent-13β)-form: | 407.3063 | 0.6426 |
| 19-O-(3-Methylbutanoyl) | | |
| 3,18,20-Filicatriene | 407.3698 | 0.2456 |
| Dimethamine | 409.2621 | 0.3979 |
| 11,13(18)-Oleanadiene | 409.3891 | 0.8221 |
| 2-(Aminomethyl)-2-propenoic acid, 9CI: N- | 410.3691 | 0.2546 |
| Eicosanoyl, Me ester | | |
| 14-Heptacosanone: Oxime | 410.4291 | 0.4024 |
| 3,4-Dihydro-3,6,8,9-tetrahydroxy-3- | 411.2146 | 0.2256 |
| methyl-1(2H)-anthracenone; (S)-form: 6- | | |
| O-(3,7-Dimethyl-2E,6-octadienyl) | | |
| Jaspic acid | 411.2922 | 0.4703 |
| Eupha-7,24-diene; (20S)-form | 411.4014 | 3.4396 |
| Austalide K | 413.2343 | 0.4441 |
| Buxupapine: N3-Me | 413.3944 | 1.7591 |
| 15-Azasterol: 24,28-Dihydro | 414.3808 | 0.2058 |
| Crispatone | 415.2571 | 2.0281 |
| Aleicide B | 416.2772 | 0.1188 |
| Pregnane-3,5,6,8,12,14,17,20-octol | 417.2526 | 0.1143 |
| 3,25-Dihydroxy-9,10-secocholesta-5,7- | 417.3434 | 0.7321 |
| dien-24-one, 9CI | | |
| Axinellamine B‡ | 419.3411 | 1.2155 |
| Phloeodictyne A; Phloeodictyne 4,7a | 420.3698 | 0.2152 |
| Lincomycin, BAN, INN: S-Oxide | 423.2245 | 0.2469 |
| 3,7,23-Trihydroxycholan-24-oic acid; | 423.3198 | 0.5671 |
| (3a,5β,7a,23R)-form: Me ester | | |
| 23,29-Imino-B (9a)-homo-19- | 423.3643 | 0.3087 |
| norstigmasta-1(10),7,9(11),23(N)-tetraen- | | |
| 3-; (3a,5a,24?)-form: 9,11-Dihydro | | |
| 5-Octacosenoic acid | 423.4172 | 0.3898 |
| Triacontane | 423.4851 | 0.5332 |
| Plakinamine A: 24,25-Dihydro | 425.3979 | 1.5100 |
| Xestosterol | 427.3874 | 1.6817 |
| Glycerol 1-alkyl ethers; Glycerol 1- | 429.3513 | 0.4824 |
| octadecyl ether: Di-Ac | | |
| 3-Hydroxymethyl-A-norgorgostane | 429.4136 | 0.3555 |
| Lankamycin, 9CI: Aglycone, 8-deoxy, O- | 431.2957 | 0.1897 |
| de-Ac | | |
| 8,11′;12,12′-Bi[1(10),7-eremophiladien-9- | 433.3164 | 1.3914 |
| one] | | |
| 3-Hydroxycholan-24-oicacid; (3a,5β)-form: | 434.3292 | 1.4844 |
| Glycine amide | | |
| 4,15,26-Triacontatriene-1,12,18,29- | 435.3289 | 1.2633 |
| tetrayne-3,28-diol; (3?,4E,15Z,26E,28?)- | | |
| form: 12,13-Dihydro(Z-) | | |
| Veratramine: 23-Deoxy, N—Ac | 436.3184 | 0.5377 |
| 4,15,26-Triacontatriene-1,12,18,29- | 437.3453 | 0.9109 |
| tetrayne-3,28-diol; (3S,15Z,28S)-form: | | |
| 4,5,26,27-Tetrahydro | | |
| Amphiasterin B3 | 439.3821 | 1.1627 |
| 8,13-Epoxy-14,15,16,19-labdanetetrol; (ent- | 441.3197 | 0.1547 |
| 8a,13R,14S)-form: 19-(3- | | |
| Methylbutanoyl) | | |
| Enterocin | 445.1189 | 0.2175 |
| Quinine, BAN: O-(2-Hydroxybenzoyl) | 445.2060 | 0.1292 |
| Antibiotic I1 | 445.2569 | 0.1012 |
| 2,3-Dihydroxyspirost-9(11)-en-12-one | 445.3040 | 0.1125 |
| Mutamicin 5 | 447.2934 | 0.2382 |
| Spirostane-1,3,5-triol | 449.3315 | 1.1368 |
| 1-Cyclopentyl-4-hexacosanone | 449.4764 | 0.2181 |
| Severibuxine | 450.3081 | 0.4942 |
| 3-Hydroxy-7,9(11),22,24-lanostatetraen- | 451.3269 | 3.2471 |
| 26,23-olide | | |
| 4-Methylaconitane-1,6,7,8,14,16,18-heptol; | 452.2552 | 0.2046 |
| (1a,5β,6β,14a,16β)-form: 14-Ketone, O6,O16, | | |
| O18-tri-Me, N-Et | | |
| 4-Methylaconitane-6,7,8,14,16,18-hexol; | 452.3056 | 1.6479 |
| (5β,6β,14a,16β)-form: O6,O14,O16,O18- | | |
| Tetra-Me, N-Et | | |
| 3,5-Dioxooctacosanoicacid | 453.3996 | 1.9017 |
| Spirosol-4-en-3-one, 9CI; (22R,25R)- | 454.3393 | 0.9274 |
| form: N—Ac | | |
| 7-[5-(Decahydro-4a-hydroxy-1,2,5,5- | 455.3303 | 0.3927 |
| tetramethyl-1-naphthalenyl)-3-methyl-2- | | |
| N-Deformyldichotamine: 10,11- | 457.2315 | 0.6811 |
| Dimethoxy, N-propanoyl | | |
| Abyssinine B | 459.2943 | 1.2960 |
| Anopteryl alcohol: 12-Tigloyl | 460.2701 | 0.8645 |
| 3′,4′,5,7-Tetrahydroxyflavone: 3′-O-β-D- | 463.0925 | 0.2008 |
| Glucuronopyranoside | | |
| 3-Deoxy-manno-oct-2-ulosonic acid, 9CI; | 463.1551 | 0.3750 |
| D-Furanose-form: 2,4,6,7,8-Penta-Ac, Me | | |
| ester | | |
| Urceolide | 463.2197 | 0.2285 |
| Spiropachysine | 463.3646 | 1.2822 |
| Lincosamine; a-Pyranose-form: 1-Thio, Me | 464.1552 | 0.7002 |
| glycoside, penta-Ac | | |
| 3,5-Acarnidine: 5,6Z-Didehydro | 464.4043 | 0.6752 |
| 2,3,14,20,25-Pentahydroxycholest-7-en-6- | 465.3246 | 0.7189 |
| one | | |
| 10,12-Hentriacontanedione | 465.4705 | 1.4889 |
| Indanomycin: 16-Deethyl | 466.3003 | 0.2089 |
| Teleocidin B1: 16-Epimer, Me ether | 466.3436 | 0.1559 |
| Cephaeline; (−)-form | 467.2937 | 2.0569 |
| Trideacetylpyripyropene A: 11-Epimer, | 468.2441 | 0.3311 |
| 7,19-dideoxy,3-Ac | | |
| Cytosine arabinoside; β-D-Furanose-form: | 468.3372 | 0.4833 |
| N4-Hexadecyl | | |
| 3,29-Dihydroxy-12-oleanen-27-oic acid; | 469.3380 | 0.5732 |
| 3a-form: 3-Ketone, 29-aldehyde | | |
| Tryptoquivaline N | 473.1871 | 0.3580 |
| Botcineric acid: 3-Ac | 473.2676 | 0.2197 |
| Mucronine C | 473.3172 | 0.2330 |
| 3,29-Dihydroxy-12-oleanen-27-oic acid | 473.3658 | 0.2675 |
| 2,29-Diamino-5,8,11,14,17,20- | 473.4145 | 0.5549 |
| triacontahexaene-3,28-diol, 9CI | | |
| Lanost-9(11)-ene-3,24,25-triol; (3β,5a,24S)- | 475.4150 | 0.2409 |
| form: 3-Me ether | | |
| Russuphelin C: 1-Me ether | 476.9480 | 0.1325 |
| Austalide H | 477.2405 | 0.0739 |
| Desoxophylloerythroetioporphyrin | 477.3036 | 0.1103 |
| Griseoviridin | 478.1682 | 0.0481 |
| 8-Dotriacontenoicacid | 479.4900 | 1.8311 |
| 2,4,6,8-Tetramethyloctacosanoic acid | 481.4898 | 0.5422 |
| Stawamycin | 482.2926 | 0.4723 |
| 2,3-Dihydroxy-24-nor-6-oxo-1,3,5(10)- | 483.3136 | 0.3535 |
| friedelatrien-29-oicacid: Me ester | | |
| Cycloheterophyllin: 2-Deoxy | 487.2192 | 0.9381 |
| Antibiotic A 2315C | 488.2376 | 0.6156 |
| Budmunchiamine L5‡ | 493.4922 | 2.0539 |
| Misenine | 495.4278 | 1.9797 |
| Lipstatin: Tetrahydro | 496.4059 | 0.1911 |
| CyclovirobuxeineI: N3,N3,N20-Tri- | 497.4057 | 0.2982 |
| Me,O-tigloyl | | |
| Tetradecanoic acid, 9CI: 2,3- | 497.4552 | 0.2035 |
| Dihydroxyheptadecenyl ester | | |
| Pseudomonic acid A: 4′,5′-Didehydro | 499.2922 | 0.1278 |
| Nemorosone | 503.3065 | 0.6934 |
| Cycloprotobuxine I: 6,7-Didehydro, N3,N20, | 503.3904 | 0.2074 |
| N20-tri-Me,N3-benzoyl | | |
| 7-Oxotetratriacontanal | 507.5223 | 1.9497 |
| 31-Methyltritriacontanoic acid | 509.5391 | 0.3267 |
| 3-Methyl-3-buten-1-o1: Triacontanoyl | 521.5231 | 1.8691 |
| Tetrahydro-2-(1-hydroxy-9-nonenyl)-5- | 523.4738 | 0.6198 |
| pentyl-3-furanol: 1′-O-Tetradecanoyl | | |
| Murrafoline C | 527.2702 | 0.3570 |
| 2,3,7,11,15-Pentahydroxy-18- | 527.3560 | 0.1094 |
| hydroxymethyl-2,6,10,14,16,20- | | |
| hexamethyl-4,8,12,16-docosatetraenoic | | |
| acid, 9CI | | |
| 9-Octadecenyl 9-octadecenoate, 9CI | 533.5258 | 0.8095 |
| Artemoin A | 551.5074 | 0.6192 |
| 3′-(8,17-Epoxy-16-oxo-12,14-labdadien- | 571.3102 | 0.7532 |
| 15-yl)-2′,4′-dihydroxy-6′- | | |
| methoxychalcone | | |
| Montecristin | 575.5089 | 0.2888 |
| Bombiprenone | 603.5416 | 1.0111 |
| 1,8,9,14-Tetrahydroxydihydro-β- | 608.2954 | 0.4141 |
| agarofuran; (1a,8β,9a)-form: 14-(3- | | |
| Pyridinecarbonyl), 9-benzoyl, 1-(2- | | |
| methylpropanoyl), 8-Ac | | |
| Jolantinine | 609.3402 | 0.9824 |
| Haliclotriol A | 609.4168 | 0.2743 |
| Dimethylmenaquinone | 609.4754 | 0.2561 |
| Maytansinol: 3-O-(3-Hydroxy-3- | 651.2745 | 0.2402 |
| methylbutanoyl), N-de-Me | | |
| Quercetin 3-glycosides; Monosaccharides: | 757.1737 | 0.2324 |
| 3-O-[3,6-Bis(4-hydroxy-E-cinnamoyl)-β- | | |
| D-glucopyranoside] | | |
| 2,3,5,7,8,9,15-Heptahydroxy-6(17),11- | 757.3037 | 0.1749 |
| jatrophadien-14-one; | | |
| (2a,3β,5a,7β,8a,9a,11E,15β)-form: 7- | | |
| Benzoyl, 2,3,5,8,9,15-hexa-Ac | | |
| Hydroxystreptomycin B | 760.3216 | 0.6499 |
| Acylsucroses: 2,3′,4′,6′-Tetrakis(3- | 763.4199 | 0.5828 |
| methylbutanoyl), 1′-(2S-methylbutanoyl) | | |
| Pregn-5-ene-3,14,20-triol; (3β,14β,20R)- | 819.4401 | 0.2765 |
| form: 3-O-[β-D-Glucopyranosyl-(1?4)-β- | | |
| D-digitalopyranoside], 20-O-β-D- | | |
| glucopyranoside | | |
| Sulfurmycin B: 1-Hydroxy | 854.3137 | 0.2808 |
| Itampolin A | 859.9955 | 0.0981 |
| 3-Phosphatidylinositol; Glycerol 1-(9,12- | 861.5496 | 0.0689 |
| octadecadienoate) 2-(9-octadecenoate) 3- | | |
| phosphoinositol | | |
| Antibiotic 1176A | 862.4856 | 0.0742 |
| Glycerol trialkanoates (diacid, | 887.7979 | 0.0556 |
| unsymmetrical); Glycerol 1,2- | | |
| dioctadecanoate 3-(9Z,12Z- | | |
| octadecadienoate) | | |
| Lyngbyabellin D | 896.2684 | 0.1104 |
| Huratoxin: 5-Deoxy, 6,7-deepoxy, 6,7- | 903.7036 | 0.2523 |
| didehydro, 20-tetracosanoyl | | |
| Quercetin 3,7-diglycosides: 3-O-[3,4- | 905.2383 | 0.2274 |
| Dihydroxy-E-cinnamoyl-(?4)-a-L- | | |
| rhamnopyranosyl-(1?2)-a-L- | | |
| arabinopyranoside], 7-O-β-D- | | |
| glucopyranoside | | |
| Periplaneta americana Pyrokinins; Pea-PK-3 | 996.6478 | 1.1928 |

TABLE 5
Chemicals present in Extract 3 as determined by DART
TOF-MS analysis and utilization of a searchable database.

| | Measured | Relative |
|---|---|---|
| Compound Name | Mass | Abundance (%) |
| Pyrrolidine, 9CI, 8CI: N-Nitroso | 101.0705 | 0.1022 |
| 2,3-Diaminopropanoicacid | 105.0757 | 1.1233 |
| 1,2-Dimethylbenzene,9CI | 107.0937 | 0.2797 |
| 1,3,6-Octatriene | 109.1061 | 0.9298 |
| N-Acetylglycine, 9CI: Amide | 117.0763 | 0.3684 |
| 2-Methylaminoacetic acid: Et ester | 118.0949 | 0.5534 |
| 2,3-Diaminopropanoicacid; (R)-form: N3- | 119.0901 | 27.9139 |
| Me | | |
| 1-Amino-3-methyl-2,3-butanediol; (S)- | 120.0949 | 2.7933 |
| form | | |
| Isopropylbenzene, 8CI | 121.1055 | 13.4246 |
| Choline: Hydroxide | 122.1111 | 1.0341 |
| Benzoic acid, 9CI, USAN | 123.0523 | 0.7548 |
| 2,3-Dimethyl-5-methylene-2-cyclopenten- | 123.0902 | 0.2841 |
| 1-one | | |
| 6-Methyl-3,5-heptadien-2-one, 9CI; (E)- | 125.1001 | 1.0320 |
| (?)-form | | |
| 3-Methyl-1-butylamine: N-Propyl | 130.1631 | 1.5347 |
| 2-Methyl-3,4-piperidinediol, 9CI | 132.0959 | 0.2228 |
| 2,4-Diaminopentanoicacid | 133.1063 | 0.5337 |
| 2-Amino-4-(aminooxy)butanoic acid, 8CI; | 135.0823 | 1.1055 |
| (±)-form | | |
| 1-Phenyl-2-propylamine | 136.1103 | 0.3780 |
| 4-Methylbenzoic acid, 9CI | 137.0655 | 0.5063 |
| 2,5,5-Trimethyl-1,3,6-heptatriene | 137.1374 | 0.1606 |
| (4-Hydroxy-3-methyl-2-butenyl)guanidine | 144.1137 | 0.3466 |
| 2,6-Diamino-4-hexenoicacid, 9CI | 145.1017 | 0.4335 |
| 1,5-Pentanediamine, 9CI: N,N,N-Tri-Me | 146.1859 | 0.1732 |
| Lysine | 147.1211 | 2.2513 |
| 5,6,7,8-Tetrahydro-4-methylquinoline | 148.1159 | 0.3391 |
| 1-(1,3-Hexadienyl)-2-vinylcyclopropane | 149.1332 | 1.6207 |
| 1-Bromo-2-propanone; (E)-form: Amide | 149.9732 | 0.0893 |
| 2,6-Decadien-4-yn-1-ol,8CI | 151.1148 | 0.7642 |
| 1-Bromo-2-propanone: Oxime | 151.9837 | 0.1643 |
| Ethyl-1,4-benzoquinone: 4-Oxime | 152.0743 | 0.2936 |
| 2-Amino-1-phenyl-1-propanol | 152.1063 | 0.0780 |
| 2-Vinyl-1,3,5-benzenetriol | 153.0586 | 1.3362 |
| Cytosine: 4-N—Ac | 154.0673 | 0.3822 |
| 1-Undecene | 155.1779 | 0.1484 |
| 3-Methyl-1,2-cyclohexanedione, 9CI: | 157.1031 | 0.1285 |
| Dioxime | | |
| 6-Propyl-3-piperidinol, 9CI; (3S,6S)- | 158.1587 | 0.4449 |
| form: N-Me | | |
| Pentanoic acid, 9CI: 2-Methylpropyl ester | 159.1312 | 0.2580 |
| Putreanine | 161.1332 | 2.2162 |
| 5,6,7,8-Tetrahydro-2,4-dimethylquinoline | 162.1303 | 0.3834 |
| Safrole | 163.0919 | 1.0758 |
| 4-Hydroxyphthalide, 8CI: Me ether | 165.0593 | 0.7317 |
| Eugenol | 165.1234 | 0.2973 |
| 6-Amino-3-methylpurine: 7-Oxide | 166.0727 | 0.2521 |
| p-Menth-1-ene-8-thiol; (S)-form | 171.1274 | 0.1345 |
| 2,2,6,6-Tetramethyl-4-piperidinone, 9CI: | 171.1591 | 0.0984 |
| Oxime | | |
| Arginine, INN, USAN; (±)-form | 175.1131 | 0.4286 |
| Muscarine‡ | 175.1478 | 0.2171 |
| methoxycoumarin | 177.0656 | 4.3193 |
| bamosamine | 178.0669 | 0.3921 |
| 4-tert-Butylphenol, 8CI: Et ether | 179.1415 | 3.3944 |
| Tecomanine | 180.1443 | 0.2838 |
| 2-Iodoethanol, 9CI, 8CI: Me ether | 186.9550 | 0.5500 |
| 6,13-Tetradecadiene-1,3-diyne | 187.1453 | 0.1017 |
| Undecanoic acid, 9CI, 8CI | 187.1769 | 0.0797 |
| 1,2,3,4-Tetrahydro-1,1,5,6- | 189.1655 | 1.1411 |
| tetramethylnaphthalene, 9CI | | |
| Glycylglycylglycine | 190.0732 | 0.2512 |
| 3-(Dimethylamino)-2,3,6-trideoxy-arabino- | 190.1479 | 0.2150 |
| hexose, 9CI,8CI; β-D-Pyranose-form: Me | | |
| glycoside | | |
| Khusitene | 191.1812 | 0.2378 |
| Elijopyrone D | 193.0867 | 3.2660 |
| alpha-phenylindol | 194.1041 | 0.4988 |
| 3,4-dihydroscopoletin | 195.0672 | 0.7026 |
| 11,12,13-Trinor-3,8-eudesmanedione | 195.1388 | 0.6109 |
| Amidinomycin | 199.1479 | 0.5906 |
| Incarvilline: 4a-Hydroxy | 200.1568 | 0.4877 |
| 1,3,5,7-Cadinatetraene, 8CI | 201.1638 | 4.4166 |
| 3,10(14)-Aromadendradiene | 203.1792 | 11.4738 |
| Dendrobates Alkaloid 203 | 204.1852 | 2.3534 |
| 3,5-Cadinadiene | 205.1947 | 8.7492 |
| Dendrobates Alkaloid 203: Dihydro (?) | 206.1942 | 1.0601 |
| 15-Nor-3-gymnomitranone | 207.1790 | 0.2470 |
| Arenaine | 208.1486 | 0.1982 |
| 3,5-Dichloro-6-methyl-1,2,4-benzenetriol | 208.9692 | 0.0814 |
| 6-Chloro-2-quinoxalinecarboxylicacid | 209.0142 | 0.0469 |
| Corypalline: N-Me | 209.1437 | 0.2170 |
| Tetraponerine 1 | 209.2042 | 0.0467 |
| 1,2,3,4-Tetrahydro-5,6,7- | 210.1157 | 0.0554 |
| trihydroxyisoquinoline: 7,8-Di-Me ether | | |
| Dodecahydro-2-methylpyrido[2,1,6- | 210.1825 | 0.0725 |
| de]quinolizine; | | |
| (2a,3a a,6a β,9a β)(3a S,9a S)- | | |
| form: N-Oxide | | |
| Tartaric acid; (2R,3R)-form: K—Na salt | 210.9622 | 0.4323 |
| 4-Methyl-1,2,6,8-tetraazacycloundeca-4,9- | 211.0764 | 0.0649 |
| diene-3,7,11-trione, 9CI | | |
| Linderazulene | 211.1219 | 0.1096 |
| 2,4-Dodecadienoicacid; (2E,4E)-form: Me | 211.1665 | 0.0379 |
| ester | | |
| Linderazulene: 2,3-Dihydro | 213.1274 | 0.3028 |
| Myrmicarin 213B | 214.1561 | 0.2380 |
| Tsitsikammafuran | 215.1417 | 1.9085 |
| Decanedioic acid, 9CI: Amide-Me ester | 216.1507 | 1.3294 |
| Verboccidentafuran | 217.1583 | 100.0000 |
| N-Deacetylkuanoniamine D; (Z)-form: 2- | 218.1636 | 17.9929 |
| Methylpropylamide | | |
| 1,3,5-Cadinatrien-10-ol; (7a H,10a)-form | 219.1745 | 86.2600 |
| Sedamine | 220.1786 | 12.4668 |
| vasicinone | 221.1904 | 10.1481 |
| 2,4,8-Decatrienoic acid; (2E,4E,8Z)- | 222.1954 | 1.3124 |
| form: 2-Methylpropylamide | | |
| Verboccidentafuran: 2-Oxo | 231.1418 | 3.7804 |
| 2-Aminoheptanedioic acid, 8CI; (±)-form: | 232.1511 | 0.8320 |
| Di-Et ester | | |
| Verboccidentafuran: 4a,5a-Epoxide | 233.1551 | 7.6377 |
| Furo[2,3-b]quinoline-4,5,7,8-tetrol, 9CI | 234.0319 | 0.1198 |
| 2-(Hydroxymethyl)-3,4,5-piperidinetriol, | 234.1665 | 2.6731 |
| 12CI; (2S,3R,4R,5S): N-Pentyl | | |
| 9-Hydroxy-4,10(14)-oplopadien-3-one | 235.1704 | 23.2049 |
| 5-(2-Butenylidene)-3-ethyl-1,2,3,4,5,7a- | 236.1746 | 3.5041 |
| hexahydro-4aH-1-pyrindine-4,4a-diol | | |
| 3,10(14),11-Germacratriene-1,9-diol | 237.1893 | 4.5243 |
| 7-Bromo-2,4-dihydroxyquinazoline | 240.9790 | 0.2164 |
| 3,5,6-Trichloro-1,2,4-benzenetriol: 2-Me | 242.9453 | 0.1801 |
| ether | | |
| Glycine: N-(2-Nitrobenzenesulfenyl),Me | 243.0369 | 0.4639 |
| ester | | |
| Vitamin H (biotin) | 245.0853 | 1.9891 |
| Mycosporin-Gly | 246.0922 | 0.3614 |
| Lycopodium Base IV | 246.1777 | 0.4171 |
| 5,9,13-Triazapentadecane-1,15-diamine | 246.2705 | 0.2407 |
| 8-Hydroxy-1,4,7(11)-guaiatrien-12,8-olide | 247.1362 | 0.7660 |
| Dodecylbenzene | 247.2501 | 0.5409 |
| 3-(3,4-Methylenedioxyphenyl)-2-propenoic | 248.1326 | 0.1400 |
| acid; (E)-form: 2-Methylpropylamide | | |
| 6-Hydroxy-4,11(13)-eudesmadien-12,8- | 249.1539 | 1.0521 |
| olide | | |
| Fungerin: N1-Me | 250.1628 | 0.4670 |
| 7,8-Dichloro-9-methyl-β-carboline | 251.0091 | 0.0829 |
| 1-Hydroxy-5,11(13)-eudesmadien-12-oic | 251.1657 | 1.1327 |
| acid | | |
| Arglecin | 252.1810 | 0.2504 |
| 2-Amino-11,15-hexadecadien-3-ol | 254.2571 | 0.1004 |
| 8-Methylpentadecanoic acid, 9CI | 257.2504 | 0.6032 |
| Murexine: Chloride | 260.1264 | 0.4379 |
| Joubertiamine; (±)-form: 2,3-Dihydro | 262.1870 | 0.1578 |
| Lycopodine; (−): Oxime | 263.2115 | 0.1688 |
| 2,6,8,10-Dodecatetraenoicacid; (all-E)- | 264.2039 | 0.6292 |
| form: 2-Hydroxy-2-methylpropylamide | | |
| Siamenol | 266.1534 | 0.3410 |
| 3,8-Dihydroxy-4(15),9,11(13)- | 267.1668 | 0.4387 |
| germacratrien-12,6-olide; (3β,6a,8a,9E)- | | |
| form: 11a,13-Dihydro | | |
| Antibiotic G 1499-2 | 268.1697 | 0.2108 |
| Calabatine | 276.1988 | 0.0985 |
| 7-Hydroxy-14,15-dinor-8(17)-labden-13- | 279.2288 | 0.2441 |
| one | | |
| 9,12-Octadecadienoic acid, 9CI; (9E,12Z)- | 281.2479 | 0.3662 |
| form | | |
| 10-Octadecenoic acid; (E)-form: Amide | 282.2787 | 0.3512 |
| Gilbertine | 283.1855 | 0.2021 |
| Pseudodistomin B | 295.2670 | 0.4583 |
| 6-Octadecenoic acid; (E)-form: Me ester | 297.2826 | 0.6637 |
| 9(11),15-Beyeradien-19-oic acid | 301.2151 | 0.4873 |
| 1-Nor-2,19-phytanediol | 301.3019 | 0.0768 |
| N2-Leucylarginine; L-L: Me ester | 302.2249 | 0.5148 |
| Tetradecanoic acid, 9CI: Anilide | 304.2715 | 0.2567 |
| Bisdemethoxycurcumin | 309.1129 | 1.5255 |
| Thehaplosin | 310.1176 | 0.6360 |
| Ephemeranthone | 311.1298 | 0.8239 |
| 11-Eicosenoic acid; (Z)-form | 311.3028 | 0.6556 |
| GrevillineA | 325.0764 | 0.2705 |
| Cneorumchromene G | 325.1480 | 0.1079 |
| 3,4,6,8-Tetrahydroxyxanthone-1- | 333.0616 | 0.1210 |
| carboxylic acid: 4,6-Di-Me ether | | |
| 4-(2,3-Dibromo-4,5-dihydroxyphenyl)-3- | 334.9282 | 0.1925 |
| buten-2-one | | |
| 3-(12-Phenyl-8-dodecenyl)phenol | 337.2543 | 1.1222 |
| 3-Greenwayodendrinol | 338.2446 | 0.1792 |
| Loesenerine | 338.2844 | 0.1948 |
| Demethoxycurcumin | 339.1225 | 3.0278 |
| Kanagawamicin | 340.1296 | 2.7440 |
| Zopfinol | 341.1480 | 1.5420 |
| Mescaline isocitrimide lactone | 350.1182 | 0.8405 |
| Eritadenine; (2R,3R)-form: 2,3-Di-Ac, | 352.1285 | 0.2049 |
| Me ester | | |
| Septicine; (R)-form: 7-Demethoxy, O6-de- | 352.1972 | 0.1341 |
| Me | | |
| Pulchellidine‡ | 352.2532 | 0.1483 |
| Pentacosanal | 367.3997 | 0.1586 |
| Mescaline citrimide | 368.1290 | 1.3949 |
| Curcumin | 369.1339 | 5.0321 |
| Tyrindoxol: S,S-Dioxide, O-sulfate | 369.9287 | 0.4521 |
| Pluracidomycin C2 | 370.0324 | 0.1252 |
| Tecleaverdoornine: Ac | 370.1380 | 11.0127 |
| (+)-Fargesin | 371.1491 | 4.9769 |
| Adenosine, 9CI, 8CI, BAN, USAN: N6- | 372.1578 | 1.6909 |
| (2-Methylbenzyl) | | |
| Sinefungin, INN, USAN: 4,5-Didehydro | 380.1631 | 0.2342 |
| Antibiotic K 252c: N6-(1- | 384.1684 | 0.0555 |
| Methylethoxy)methyl | | |
| Sesangolin | 385.1262 | 0.1328 |
| 3-Bromo-8,13-epoxy-14-labden-6-ol; (ent- | 385.1897 | 0.2506 |
| 3β,6β,8a,13S)-form | | |
| Nocardicin G | 386.1426 | 0.1265 |
| 3,3′,4,4′,7′,8-Hexahydroxylignan-9,9′- | 391.1469 | 0.1535 |
| olide; (7′R,8S,8′R)-form: 3,3′-Di-Me | | |
| ether | | |
| 3-Hydroxy-6-oxocholan-24-oic acid | 391.2922 | 1.1109 |
| 3,7-Dihydroxycholan-24-oic acid; | 393.2990 | 0.1039 |
| (3β,5β,7β)-form | | |
| 14-Methyl-9,19-cycloergost-24(28)-en-3-ol | 413.3874 | 0.1587 |
| 1,1,2-Tribromo-6-hydroxy-1-octen-3-one; | 418.9013 | 0.2246 |
| (±)-form: Ac | | |
| 9′-Hydroxy-9′-apo-e-caroten-3-one | 419.2978 | 0.1136 |
| 13,17,19-Villanovanetriol; (ent-13β)-form: | 421.3362 | 0.2047 |
| 19-O-(3-Methylpentanoyl) | | |
| 8,11′;12,12′-Bi[1(10),7-eremophiladien-9- | 433.3204 | 0.3722 |
| one] | | |
| 4,15,26-Triacontatriene-1,12,18,29- | 435.3334 | 0.6170 |
| tetrayne-3,28-diol; (3?,4E,15Z,26E,28?)- | | |
| form: 12,13-Dihydro(Z-) | | |
| 2-Tridecyl-2-heptadecenal | 435.4525 | 0.1001 |
| 3-Hentriacontene; (Z)-form | 435.5016 | 0.0634 |
| Philanthotoxin 433 | 436.3316 | 0.8459 |
| 2,3,5,6-Tetrabromo-1,4-benzenediol, 9CI: | 436.7899 | 0.1097 |
| Mono-Me ether | | |
| Rubrolide E: 3″,5″-Dibromo | 436.9403 | 0.1163 |
| Stolonic acid A: 25,26-Dihydro | 437.3302 | 0.0975 |
| 1,5-Dihydro-5-hydroxy-3-methyl-2H- | 438.1694 | 0.2300 |
| pyrrol-2-one, 9CI; (R): O-[β-D- | | |
| Glucopyranosyl-(1?3)-β-D- | | |
| glucopyranoside] | | |
| Isoleucylamiclenomycylglutamic acid: | 438.2683 | 0.1466 |
| Amide | | |
| Teleocidin B1: N-De-Me | 438.3194 | 0.1408 |
| Alkaloid LO3 | 440.3913 | 0.1419 |
| 1,2-Bis[2-(2,4,5- | 441.2333 | 0.3112 |
| trimethoxyphenyl)ethenyl]cyclobutane | | |
| 1,2,4,5-Pentanetetrol; (2R,4R)-form: 1,5- | 445.0940 | 0.1275 |
| Bis(4-methylbenzenesulfonyl) | | |
| 2,3-Dihydroxyspirost-9(11)-en-12-one | 445.2941 | 0.2620 |
| Pseurotins; Pseurotin E | 446.1371 | 0.1222 |
| Narceine | 446.1877 | 0.0939 |
| Clavulones | 447.2346 | 0.1100 |
| 16-Kaurene-3,7,18-triol; (ent-3β,7a)-form: | 447.2843 | 0.1573 |
| Tri-Ac | | |
| CNS 2103 | 447.3348 | 0.1626 |
| Stigmast-5-ene-3,7,22-triol; (3β,7a,22R, | 447.3832 | 0.1062 |
| 24R)-form | | |
| Spirostane-1,3,5-triol | 449.3294 | 0.6112 |
| Chaksine | 451.3110 | 0.1353 |
| β-Rubromycin: 3′-Hydroxy | 553.0969 | 0.1739 |
| 2-Amino-3-hydroxy-15-methyl-4- | 590.4502 | 0.1220 |
| hexadecene-1-sulfonic acid; (2S,3R,4E)- | | |
| form: N-(2R-Hydroxy-13- | | |
| methyltetradecanoyl) | | |
| Ferensimycin B | 643.4424 | 0.1640 |
| Delphinidin 3-glycosides: 3-O-[3,4,5- | 660.1292 | 0.4060 |
| Trihydroxybenzoyl-(?2)-6-O-acetyl-β-D- | | |
| galactopyranoside] | | |
| 8,14-Epoxy-3,11,12-trihydroxypregnan-20- | 669.3829 | 0.3577 |
| one; (3β,8β,11a,12β,14β,17a)-form: 3-O- | | |
| [6-Deoxy-3-O-methyl-β-D-allopyranosyl- | | |
| (1?4)-2,6-dideoxy-3-O-methyl-β-D- | | |
| arabino-hexopyranoside] | | |
| Enterobactin | 670.1454 | 0.1320 |
| Oxazolomycin: 16S-Methyl | 670.3736 | 0.1326 |
| Pacidamycin D | 712.3141 | 0.1405 |
| Betanidin: 5-O-[β-D-Glucopyranosyl- | 713.1946 | 0.1022 |
| (1?2)-β-D-glucopyranoside] | | |
| Cycloartane-3,24,25-triol; (3β,24S): 25- | 713.6372 | 0.0648 |
| Me ether, 3-hexadecanoyl | | |
| Hexadellin B: N20-Me | 727.9336 | 0.1286 |
| 3,3′,4,4′,5,5′,9-Heptahydroxy-7,9′- | 745.2838 | 0.2778 |
| epoxylignan; (7S,8R,8′R)-form, 3,3′,5,5′- | | |
| Tetra-Me ether, 4,4′-di-O-β-D- | | |
| glucopyranoside | | |
| Gabonine | 745.4202 | 0.3427 |
| Antibiotic X 14952B | 780.4883 | 0.3587 |
| 1,3,24-Trihydroxy-24- | 845.4963 | 0.2354 |
| (hydroxymethyl)cycloartan-28-oic acid; | | |
| 31-O-β-D-Glucopyranoside, 28-O-β-D- | | |
| glucopyranosyl ester | | |
| Dammar-24-ene-3,6,12,20-tetrol; | 869.5190 | 0.5987 |
| (3β,6a,12β,20S)-form: 6-O-[2-Butenoyl- | | |
| (?6)-β-D-glucopyranoside], 20-O-β-D- | | |
| glucopyranoside | | |
| Leucocerebrosides; !Leucocerebroside B | 870.7322 | 0.0993 |
| Pradimicin L | 871.2725 | 0.1356 |
| 6-O-β-D-Galactofuranosyl-D-galactose, | 871.3600 | 0.1067 |
| 9CI; a-Pyranose-form: 1,2,3,4- | | |
| Tetrabenzyl,2′,3′,5′,6′-tetra-Ac | | |
| Avermectin B1a: 5-Ketone | 871.4796 | 0.2524 |
| Callatostatins; Callatostatin5 | 882.3806 | 0.4727 |
| Antibiotic SPA 6952A | 894.6034 | 0.3009 |
| 3,14,16-Trihydroxycard-20(22)-enolide; | 901.4383 | 0.1113 |
| (3β,5β,14β,16β)-form: 16-Ac, 3-O-[β-D- | | |
| glucopyranosyl-(1?6)-β-D-glucopyranosyl- | | |
| (1?4)-2,6-dideoxy-3-O- | | |
| Dotriacontanoicacid: Triacontanyl ester | 901.9593 | 0.1933 |

### C. Chemical Features and Biological Activities of Turmeric Extracts and Curcuminoid Standards

The three standardized turmeric extracts were fingerprinted using DART TOF-MS (FIG. 1). The distribution by mass (m/z [M+H]⁺) of the curcuminoids and turmerones in the different extracts are shown in FIG. 1 with their relative abundances. Both Extract 1 and 2 possessed over 120 chemical entities each in the m/z [M+H]⁺ range of 100to 1000 amu. The unidentified species include some MS-generated fragments and isotopes of parent compounds. Extracts 1 and 3 were chosen for further biological analysis because they represent extracts with the greatest difference in the ratios of curcuminoids to turmerones (see also Table 6). Extract 1 is enriched in the major curcuminoids, Cur, DMC, BDMC and THC in an approximate DART TOF-MS defined ratio of 20:4:1:0.01. This extract contains 72% curcuminoids and 28% turmerones based on DART TOF-MS composition. In contrast, Extract 2 lacks detectable THC, and possesses about 22% of the major curcuminoids and 78% turmerones. Extract 3, a neat ethanolic extract of Extract 2, is highly enriched in turmerones (>97%; See FIG. 1), and contains very low levels (<2%) of the major curcuminoids. Table 6 summarizes the key curcuminoids and turmerones present in these extracts. The turmerones, xanthorrhizol, ar-turmerone, and zingiberene were particularly abundant in Extracts 2 and 3. Extract 1, enriched in curcuminoids, also has significant amounts (1-10% composition) of the three turmerones, xanthorrhizol, ar-turmerone, and zingiberene.

TABLE 6
Chemical composition of Turmeric extracts and the Curcumin standard. The
chemical class, measured molecular mass (DART TOF-MS) and normalized relative
abundances of the curcuminoids and turmerone chemistries present in the turmeric Extract 1,
Extract 2, and Extract 3 as well as a Curcumin standard. Note that the Curcumin standard is a
mixture of curcuminoids and contains traces of turmerones.

| | | | Normalized Abundance (%) | | | |
|---|---|---|---|---|---|---|
| | Chemical | Measured | Curcumin | | | |
| Chemical Name | Class | Mass | Std | Extract 1 | Extract 2 | Extract 3 |
| Cucumin | curcuminoid | 369.1 | 81.2 | 66.8 | 17.3 | 0.6 |
| Demethoxycurcumin | curcuminoid | 339.1 | 12.5 | 11.9 | 3.5 | NP |
| Bisdemethycurcumin | curcuminoid | 309.1 | 1.9 | 3.3 | 1.1 | NP |
| Tetrahydrocurcumin | curcuminoid | 373.2 | NP | 0.5 | NP | NP |
| Ar-Turmerone | turmerone | 217.2 | 2.0 | 10.1 | 40.1 | 49.3 |
| Xanthorrhizol | turmerone | 219.2 | 2.3 | 6.7 | 34.6 | 38.7 |
| Zingiberene | turmerone | 205.2 | NP | 0.7 | 3.5 | 11.4 |

NP = Not present.

### D. Identification of Bioactive Compounds in Turmeric Extracts

Tables 7 and 8 show the known compounds in turmeric that are likely contributors of Aβ aggregation and APP secretion from SweAPP N2a cells. Tables 7 and 8 list the compound names, molecular masses, LogP, CLogP−(N+O), and tPSA values, as well as the percent relative abundances, and weights per 100 mg dose of extract. The parameters LogP, CLogP−(N+O), and tPSA are common parameters to monitor for determining the ability of a chemical to cross the BBB (H. Pajouhesh and G. R. Lenz, 2005. Medicinal chemical properties of successful central nervous system drugs, NeuroRx. 2:541-553). In particular, a chemical is likely to cross the BBB if the value for LogP is between 1.5 and 4.0, CLogP−(N+O) (the number of Nitrogens [N] and Oxygens [O] present in a compound) is less than zero, and tPSA is less than or equal to 80. A “/” between two compound names indicates that one of the two compounds is present. For example, in Table 7, “decadienal/santolina” indicates that the compound is decadienal or santolina epoxide.

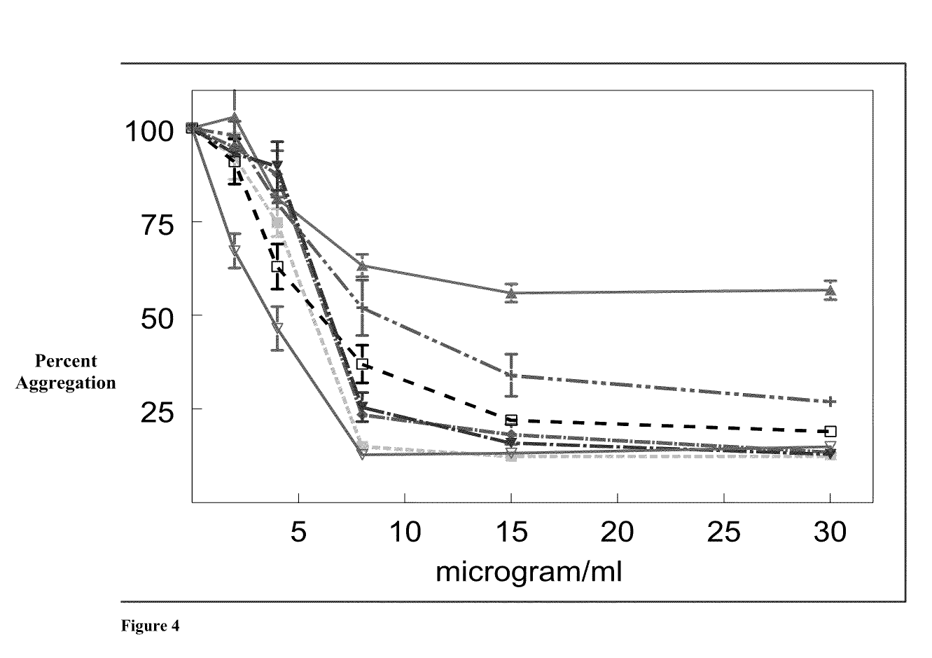

Compounds such as curcuminoids and turmerones are typically identified as the components of turmeric that contribute to anti-aggregation of Aβ activity as well as other biological activity such as the reduction of inflammation and cancer therapies (I. Chattopadhyay, K. Biswas, U. Bandyopadhyay and R. Banerjee, 2004. Turmeric and curcumin: Biological actions and medicinal applications, Curr. Sci. 87:44-52; S. Bengmark, 2006. Curcumin, an atoxic antioxidant and natural NFkappaB, cyclooxygenase-2, lipooxygenase, and inducible nitric oxide synthase inhibitor: a shield against acute and chronic diseases, JPEN J. Parenter. Enteral Nutr. 30:45-51; H. Boon and J. Wong, 2004. Botanical medicine and cancer: a review of the safety and efficacy, Exp. Opin. Pharmacother. 5:2485-2501). The curcuminoids identified as active inhibitors of Aβ aggregation here include bisdemethoxycurcumin and curcumin. Based on in vitro data (FIG. 4), demethoxycurcumin and tetrahydrocurcumin are not likely contributing to the inhibition of Aβ aggregation as effectively as curcumin and bisdemethoxycurcumin. Echinaxanthol is commonly found in Echinacea purpurea (C. Hall, 2008. Dictionary of Natural Products on DVD, (Chapman & Hall: Dictionary of Natural Products on DVD—Version 16:2, CRC Press, Boca Raton, Fla., Dictionary of Natural Products.) which is a botanical known to possess activity against depression and other central nervous system targets (V. A. Kurkin, A. V. Dubishchev, V. N. Ezhkov, I. N. Titova and E. V. Avdeeva, 2006. Antidepressant activity of some phytopharmaceuticals and phenylpropanoids, Pharmaceutical chemistry journal. 40:614-619). Eugenol, an essential oil component of cloves and cinnamon, has also recently been identified as a potential therapeutic for Alzheimer's disease because it reduces neurotoxicity associated with Aβ insult in vitro (Y. Irie, 2006. Effects of Eugenol on the Central Nervous System: Its Possible Application to Treatment of Alzheimer's Disease, Depression, and Parkinson's Disease, Current Bioactive Compounds. 2:57-66). Eugenol also increases the expression of brain-derived neurotropic factor, which is essential for antidepressant function (Y. Irie, 2006. Effects of Eugenol on the Central Nervous System: Its Possible Application to Treatment of Alzheimer's Disease, Depression, and Parkinson's Disease, Current Bioactive Compounds. 2:57-66). The other compounds identified as active inhibitors of Aβ aggregation have not previously been reported to have this specific activity.

TABLE 7
Chemicals in Turmeric extracts identified as the active inhibitors of Aβ
aggregation. Molecular properties for crossing blood-brain-barrier; LogP = 1.5-4.0, CLogP -
(N + O) > 0, tPSA ≦ 80.

| | | | | | Relative | | |
|---|---|---|---|---|---|---|---|
| | Mol. | | CLogP | | Abund. | Wt (μg) | % Weight |
| Compound Name | Mass | LogP | (N + O) | tPSA | (%) | per 100 mg | of Sample |
| decadienal/ | 151.120 | 2.66 | 2.27 | 17.07 | 0.76-1.05 | 135-322 | 0.135-0.322 |
| santolina epoxide | | | | | | | |
| eugenol | 164.084 | 2.57 | 1.40 | 29.46 | 0.14-0.28 | 62-127 | 0.062-0.127 |
| methoxycoumarin | 176.120 | 3.26 | 1.69 | 17.07 | 3.42-4.63 | 767-1500 | 0.767-1.500 |
| Bamosamine | 177.100 | −2.25 | −6.91 | 89.79 | 0.39-0.71 | 70-217 | 0.07-0.217 |
| Elijopyrone D | 192.115 | 2.30 | 1.21 | 26.30 | 0.61-3.27 | 270-580 | 0.270-0.580 |
| Vitamin H (biotin) | 244.088 | −0.12 | −6.33 | 78.43 | 1.99-5.65 | 353-2484 | 0.353-2484 |
| Echinaxanthol | 254.188 | 1.80 | −1.83 | 57.53 | 0.20-1.00 | 88-305 | 0.088-0.305 |
| Bisdemethoxy- | 308.105 | 2.81 | −1.45 | 74.60 | 1.50-4.93 | 294-1513 | 0.294-1.513 |
| curcumin | | | | | | | |
| Daphniyunnine E | 341.199 | 0.47 | −3.30 | 57.61 | 0.30-0.44 | 132-134 | 0.132-0.134 |
| Epierythro- | 350.100 | −1.64 | −7.36 | 133.52 | 0.60-0.75 | 184-331 | 0.184-0.331 |
| stominol | | | | | | | |
| Curcumin | 368.126 | 2.56 | −3.75 | 93.06 | 5.04-100 | 918-43923 | 0.918-43.923 |

The active inhibitors of Aβ aggregation identified in Extract 1 include decadienal/santolina epoxide, eugenol, methoxycoumarin, Bamosamine, Elijopyrone D, Echinaxanthol, Bisdemethoxy-curcumin, Daphniyunnine E, Epierythro-stominol, and Curcumin. The active inhibitors of Aβ aggregation identified in Extract 2 include decadienal/santolina epoxide, eugenol, vitamin H, Echinaxanthol, Bisdemethoxy-curcumin, and Curcumin. The active inhibitors of Aβ aggregation identified in Extract 3 include eugenol, methoxycoumarin, bamosamine, Elijopyrone D, vitamin H, bisdemethoxycurcumin, and curcumin.

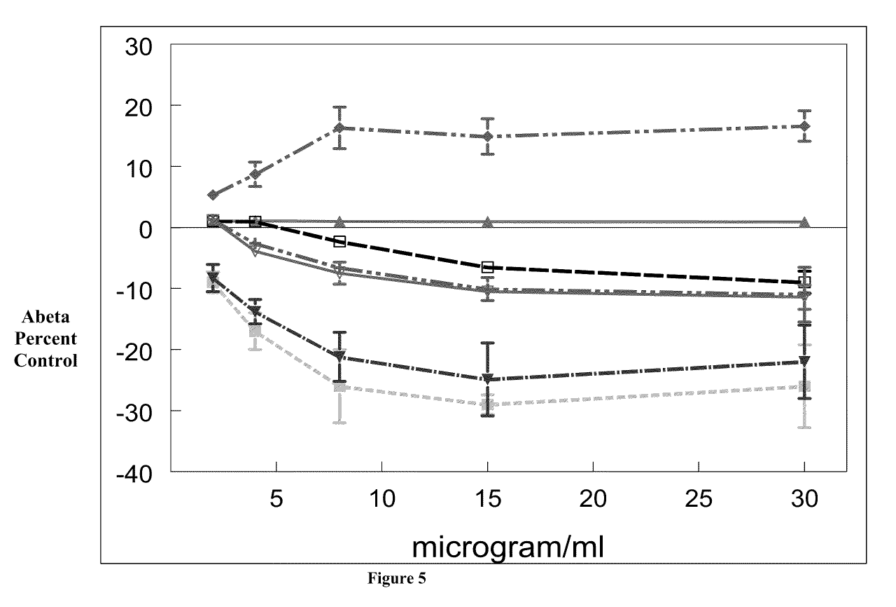

Table 8 presents the compounds in Extracts 1, 2, and/or 3 that contribute to the inhibition of APP secretion from SweAPP N2a cells. Coumarin derivates have been shown to inhibit the β-secretase enzyme (L. Piazzi, A. Cavalli, F. Colizzi, F. Belluti, M. Bartolini, F. Mancini, M. Recanatini, V. Andrisano and A. Rampa, 2008. Multi-target-directed coumarin derivatives: hAChE and BACE1 inhibitors as potential anti-Alzheimer compounds, Bioorg. Med. Chem. Lett. 18:423-426). The turmeric extracts here contain methoxycoumarin and ethoxycoumarin, as well as scopoletin and other flavonoids that have been identified as possessing secretase inhibitory activity. From in vitro data presented here (FIG. 5), three of the curcuminoid standards inhibited APP secretion (curcumin, demethoxycurcumin, bisdemethoxycurcumin) while tetrahydrocurcumin enhanced APP secretion from SweAPP N2a cells. Piperine is a compound traditionally isolated from peppers that has been shown to increase curcumin bioavailability (uptake into cells) as well as modulate the permeability of cell membranes (G. Shoba, D. Joy, T. Joseph, M. Majeed, R. Rajendran and P. S. Srinivas, 1998. Influence of piperine on the pharmacokinetics of curcumin in animals and human volunteers, Planta Med. 64:353-356; A. Khajuria, N. Thusu and U. Zutshi, 2002. Piperine modulates permeability characteristics of intestine by inducing alterations in membrane dynamics: influence of brush border membrane fluidity, ultrastructure and enzyme kinetics, Phytomedicine. 9:224-231). Both of these intrinsic properties of piperine could be contributing to the inhibition of APP secretion by increasing the concentration of the active components of the extract in the cell membrane. Although many alkaloids have been suggested to be useful for the treatment of Alzheimer's disease and other neurodegenerative disorders, the alkaloids in Table 8, have not previously been reported to have specific activity inhibiting APP secretion from cells (Z. U. Babar, A. Athar and M. H. Meshkatalsadat, 2006. New bioactive steroidal alkaloids from Buxu hyrcana, Steroids. 71:1045-1051; C. Garino, N. Pietrancosta, Y. Laras, V. Moret, A. Rolland, G. Quelever and J. L. Kraus, 2006. BACE-1 inhibitory activities of new substituted phenyl-piperazine coupled to various heterocycles: chromene, coumarin and quinoline, Bioorg. Med. Chem. Lett. 16:1995-1999).

TABLE 8
Chemicals in Turmeric extract identified as the active inhibitors of APP
secretion. Molecular properties for crossing blood-brain-barrier; LogP = 1.5-4.0, CLogP -
(N + O) > 0, tPSA ≦ 80.

| | | | | | Relative | | |
|---|---|---|---|---|---|---|---|
| Compound | Mol. | | CLogP - | | Abund. | Wt (μg) | % Weight of |
| Name | Mass | LogP | (N + O) | tPSA | (%) | per 100 mg | Sample |
| lysine | 146.106 | −1.15 | −7.42 | 89.34 | 0.31-2.25 | 108-400 | 0.108-0.400 |
| methoxycoumarin | 176.048 | 1.02 | −1.12 | 35.53 | 3.41-4.63 | 767-1500 | 0.767-1.500 |
| Bamosamine | 177.059 | 2.96 | 2.64 | 12.36 | 0.39-0.71 | 70-217 | 0.070-0.217 |
| ethoxycoumarin | 190.065 | 1.36 | −0.59 | 35.53 | 0.14-0.49 | 61-150 | 0.061-0.150 |
| α-phenylindol | 193.096 | 3.31 | 3.23 | 12.03 | 0.12-0.50 | 52-125 | 0.052-0.125 |
| 3,4- | 194.067 | 1.33 | −3.08 | 55.76 | 0.40-1.95 | 178-599 | 0.178-0.599 |
| dihydroscopoletin | | | | | | | |
| vasicinone | 202.067 | 0.53 | −4.48 | 52.90 | 0.35-11.47 | 156-2037 | 0.156-2.037 |
| 11-Epileontidane | 220.197 | 1.28 | 0.16 | 6.48 | 0.24-10.14 | 105-1802 | 0.105-1.802 |
| Echinaxanthol | 244.077 | 1.83 | −1.26 | 63.60 | 1.99-5.65 | 353-2484 | 0.353-2.484 |
| Methoxyflavanone | 254.102 | 3.38 | 0.12 | 35.53 | 0.20-0.99 | 88-305 | 0.088-0.305 |
| Aconitic acid, | 258.101 | 0.81 | −3.76 | 78.90 | 0.33-0.48 | 144-146 | 0.144-0.146 |
| triethyl ester | | | | | | | |
| 5,7-dimethoxy- | 284.114 | 2.55 | −0.54 | 44.76 | 0.48-0.62 | 190-212 | 0.190-0.212 |
| flavanone | | | | | | | |
| piperine | 285.142 | 2.78 | −0.69 | 38.77 | 0.28-1.07 | 87-468 | 0.087-0.468 |
| Bisdemethoxy- | 308.109 | 2.81 | −1.45 | 74.60 | 1.50-4.93 | 294-1513 | 0.294-1.513 |
| curcumin | | | | | | | |
| Ephemeranthone | 310.125 | 2.10 | −1.14 | 63.60 | 0.81-2.17 | 357-667 | 0.357-0.667 |
| neohesperidose | 326.119 | −3.26 | −13.19 | 169.30 | 0.94-1.11 | 289-491 | 0.289-0.491 |
| Demethoxy- | 338.117 | 2.69 | −2.60 | 83.83 | 3.02-17.80 | 536-5773 | 0.536-5.773 |
| curcumin | | | | | | | |
| Zopfinol | 340.142 | 3.74 | −1.09 | 80.92 | 1.24-1.86 | 274-571 | 0.274-0.571 |
| Daphniyunnine E | 341.154 | 2.62 | −2.49 | 59.00 | 0.30-0.44 | 132-134 | 0.132-0.134 |
| dehydroagastanol | 342.176 | 3.60 | 1.36 | 66.76 | 0.28-0.33 | 101-123 | 0.101-0.123 |
| Curcumin | 368.128 | 2.56 | −3.75 | 93.06 | 5.04-100 | 918-43923 | 0.918-43.923 |
| (+)-Fargesin | 370.144 | 2.49 | −3.43 | 55.38 | 1.28-1.77 | 300-561 | 0.300-0.561 |

The active inhibitors of APP secretion identified in Extract 1 include lysine, Bamosamine, ethoxycoumarin, alpha-phenylindol, 3,4-dihydroscopoletin, vasicinone, 11-Epileontidane, Echinaxanthol, Methoxyflavanone, Aconitic acid, triethyl ester, 5,7-dimethoxy-flavanone, piperine, Bisdemethoxy-curcumin, Ephemeranthone, neohesperidose, Demethoxycurcumin, Zopfinol, Daphniyunnine E, dehydroagastanol, Curcumin and (+)-Fargesin. The active inhibitors of APP secretion identified in Extract 2 Echinaxanthol, Bisdemethoxycurcumin, Ephemeranthone, Demethoxycurcumin, Zopfinol, Curcumin and (+)-Fargesin. The active inhibitors of APP secretion identified in Extract 3 include lysine, Bamosamine, alpha-phenylindol, 3,4-dihydroscopoletin, 11-Epileotidane, Echinaxanthol, Methoxyflavanone, Aconitic acid, triethyl ester, 5,7-dimethoxy-flavanone, Bisdemethoxy-curcumin, Ephemeranthone, neohesperidose, Demethoxycurcumin, Zopfinol, dehydroagastanol, Curcumin and (+)-Fargesin.

D. Interaction Matrices of Curcuminoids with Turmeric Extracts

Experiments were conducted to determine if synergistic, antagonistic, or additive effects were present between the individual compounds and the turmeric Extracts 1, 2, and 3. It was found that the effects of the individual curcuminoids with Extract 1 are additive in all cases as summarized in Table 9. The greatest reductions in experimental IC₅₀ values for Aβ aggregation were observed when Extract 1 was added to Extract 3, the extract rich in turmerones (>450 fold decrease in IC₅₀) and DMC (>110-fold decrease in IC₅₀; Table 9). Extract 2 and Cur, THC and BDMC showed only ca. 2-8-fold improvement in activity of Extract 1. Since the effects were only slightly additive, even at curcuminoid concentrations as high as 30 μg mL⁻¹, none of the individual curcuminoids are significantly more effective in blocking Aβ aggregation in vitro than Extract 1.

TABLE 9
Interaction matrices between Extract 1 and Extracts 2, 3 and
curcuminoid standards. The individual (extract or standard alone),
calculated theoretical, and experimental (Extract 1 plus interaction
extract or standard) IC₅₀ values (μg mL⁻¹) are provided.

| | IC₅₀ (μg mL⁻¹) | | | |
|---|---|---|---|---|
| Extract | Individual | Theoretical | Experimental | Effect |
| Extract 1 | 4.6 | 4.6 | 4.6 | — |
| Extract 2 | 40.0 | 4.6 | 5.4 | Additive |
| Extract 3 | 1682.0 | 5.1 | 3.5 | Additive |
| Cur | 41.4 | 4.6 | 4.9 | Additive |
| DMC | 575.2 | 4.6 | 6.7 | Additive |
| BDMC | 8.7 | 4.6 | 3.7 | Additive |
| THC | 35.5 | 4.6 | 4.9 | Additive |

### E. Alzheimer Pathologies in Brain of Tg2576 Mice

1. Oral Administration of Extract 1 Reduces Cerebral Amyloidosis in Tg2576 Mice

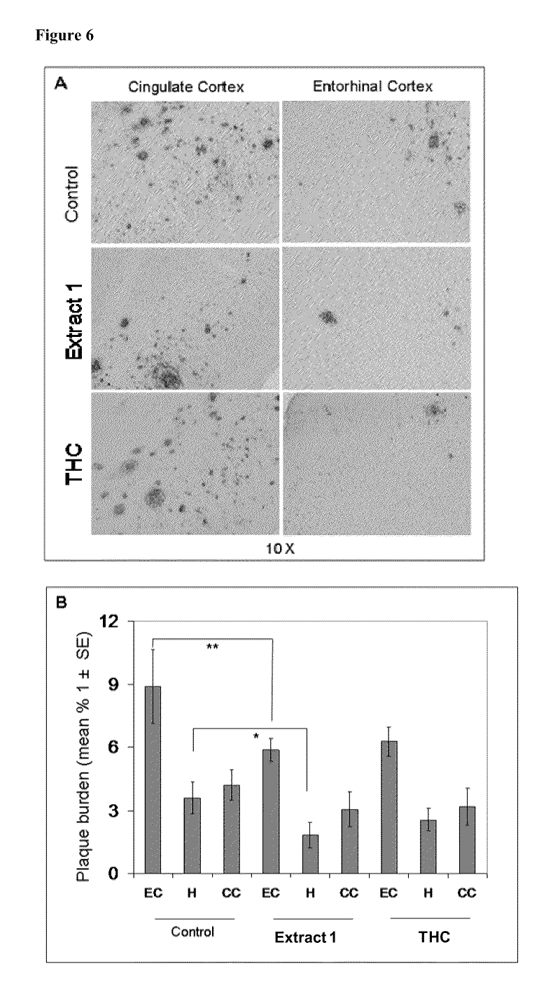

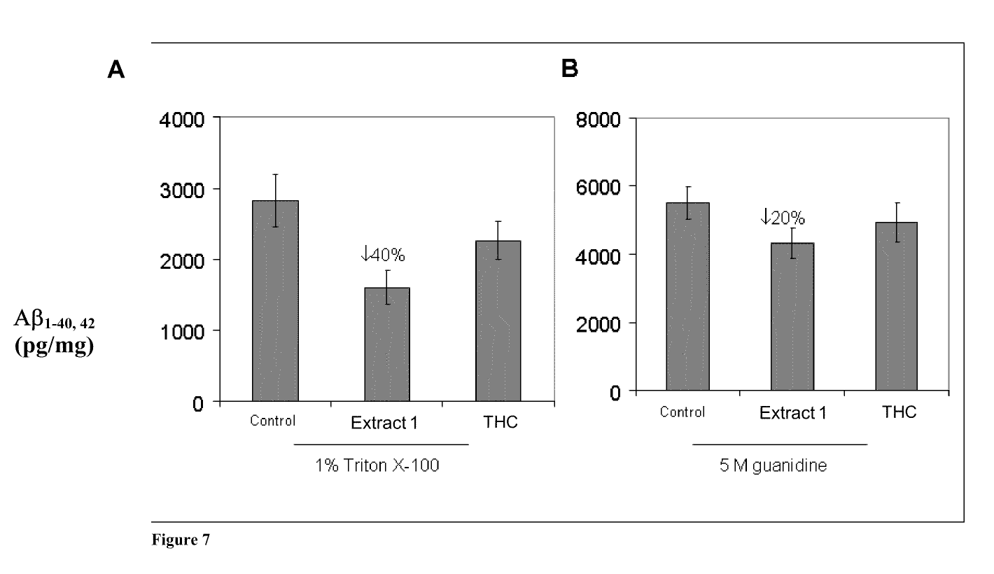

To determine whether oral administration of turmeric Extract 1 and THC could have similar anti-amyloidogenic effects in vivo as identified in vitro (above; see FIG. 4), Tg2576 mice were orally treated with 0.07% (w/w) Extract 1 supplemented or 0.07% (w/w) supplemented THC diet at 8 months of age for 6 months. As shown in FIG. 6, we found that Extract 1 treatment reduced Aβ deposition in these mice to a greater degree than THC. Image analysis of micrographs from Aβ antibody (4G8) stained sections reveals that plaque burdens were significantly reduced throughout the entorhinal cortex and hippocampus (P<0.01, P<0.05; FIG. 6B) with Extract 1 treatment compared to THC and untreated controls. To verify the findings from these coronal sections, we analyzed brain homogenates for Aβ levels by ELISA. Again, Extract 1 oral treatment markedly decreased both soluble and insoluble forms of Aβ_{1-40, 42} (FIG. 7A and B), while THC showed had much lower activity against plaque accumulation. Taken together, the above data confirm an oral route of administration of Extract 1 provides effective attenuation of amyloid pathology.

2. Oral Administration of Extract 1 Reduces Tau Hyper-Phosphorylation in Tg2576 Mice

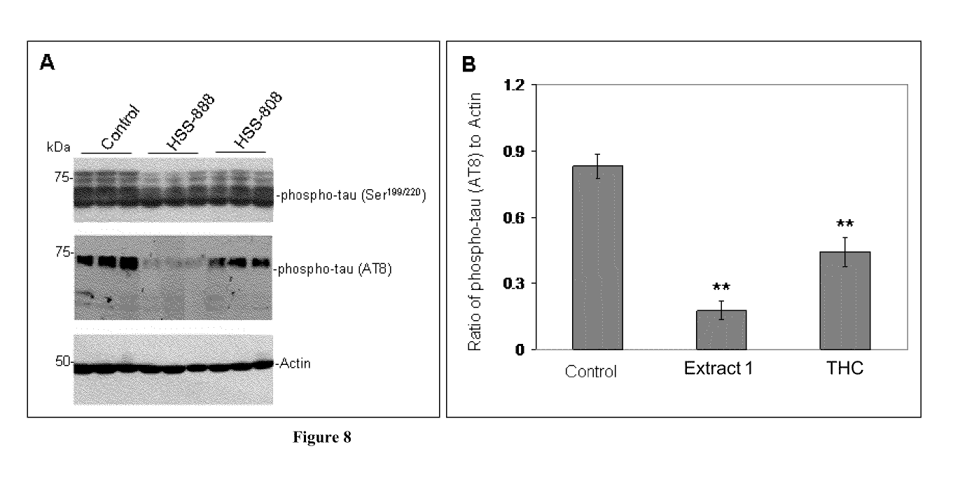

To investigate the possibility that Extract 1 may also affect tau physiology, we analyzed anterior quarter brain homogenates from the treated mice by Western blot. FIG. 8 represents soluble fractions of phosphorylated tau detected in the homogenates of the treatment groups and their control mice by both Ser^{199/220} and AT8 antibodies. The Tg2576 mice orally treated with Extract 1 and THC show decreased phosphorylated tau protein, with Extract 1 being most effective (P<0.01, FIG. 8A, B) and reducing hyper-phosphorylation by 82% compared to control. The THC treated mice showed a ca. 40% reduction in tau phosphylation over untreated control animals. Previous studies have suggested that soluble hyper-phosphorylated isoforms are ultimately the neurotoxic species of tau (D. M. Dickey, D. R. Flora, P. M. Bryan, X. Xu, Y. Chen and L. R. Potter, 2007. Differential regulation of membrane guanylyl cyclases in congestive heart failure: natriuretic peptide receptor (NPR)-B, Not NPR-A, is the predominant natriuretic peptide receptor in the failing heart, Endocrinology. 148:3518-3522; K. S. Kosik and H. Shimura, 2005. Phosphorylated tau and the neurodegenerative foldopathies, Biochim. Biophys. Acta. 1739:298-310). Accordingly, both Extract 1 and THC may afford protection from the effects of these toxic tau isoforms.

3. Oral Administration of Extract 1 Enhances Th2 Cellular Immunity in Tg2576 Mice

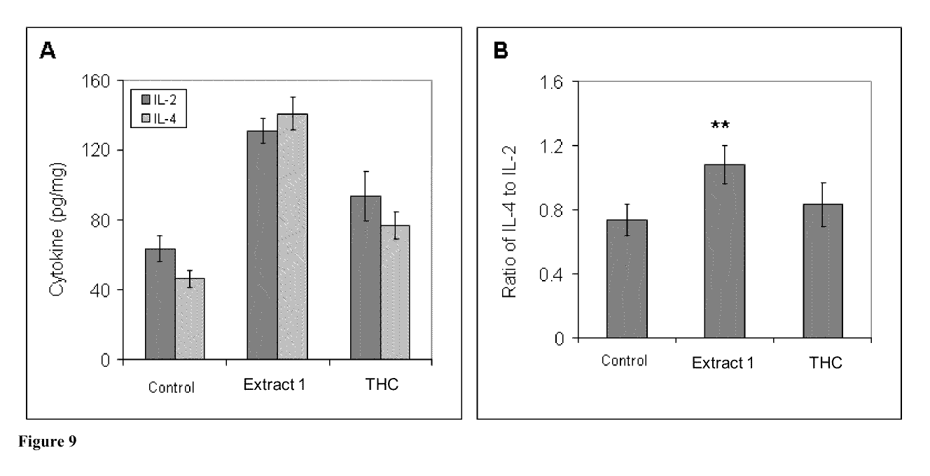

As previous studies have established the ability of curcumin to both suppress an inflammatory immune response and promote the shift from Th1 to Th2 immunity, (X. Zhang, Y. Xu, J. Zhang, J. Wu and Y. Shi, 2005. Structural and dynamic characterization of the acid-unfolded state of hUBF HMG box 1 provides clues for the early events in protein folding, Biochemistry. 44:8117-8125; S. S. Kang, T. Kwon, D. Y. Kwon and S. I. Do, 1999. Akt protein kinase enhances human telomerase activity through phosphorylation of telomerase reverse transcriptase subunit, J. Biol. Chem. 274:13085-13090) we investigated the ability of Extract 1 and THC to mediate these effects in Tg2576 mice. Following sacrifice of both treatment and control groups, primary cultures of splenocytes were established from these mice and stimulated for 24 h with anti-CD3 antibody. As illustrated in FIG. 9(A & B), the IL-4 to IL-2 cytokine profile of Extract 1-treated mice was significantly increased (P<0.001) compared to untreated control animals as well as THC-treated animals. Altogether, these data suggest that Extract 1 may be an effective immunomodulator and consequently capable of reducing inflammation while mediating clearance of Aβ.

We claim:

1. A turmeric extract comprising at least one compound selected from the group consisting of 25 to 500 μg bamosamine, 25 to 750 μg echinaxanthol, 100 to 3,000 μg bisdemethoxycurcumin, 50 to 500 μg daphniyunnine E and 500 to 75,000 μg curcumin per 100 mg of extract.

2. The turmeric extract of claim 1, further comprising at least one compound selected from the group consisting of 50 to 500 μg decadienal/santolina epoxide, 10 to 500 μg eugenol, 0200 to 3,000 μg methoxycoumarin, 100 to 2,000 μg elijopyrone D, 100 to 5,000 μg vitamin H (biotin), and 50 to 500 μg epierythrostominol per 100 mg of extract.

3. The turmeric extract of claim 1, further comprising at least one compound selected from the group consisting 50 to 1,000 μg lysine, 100 to 3,000 μg methoxycoumarin, 10 to 500 μg ethoxycoumarin, 10 to 500 μg α-phenylindol, 50 to 1,000 μg 3,4-dihydroscopoletin, 50 to 5,000 μg vasicinone, 50 to 5,000 μg 11-epileontidane, 10 to 500 μg methoxyflavanone, 50 to 500 μg aconitic acid triethyl ester, 50 to 500 μg 5,7-dimethoxyflavanone, 10 to 1,000 μg piperine, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

4. The turmeric extract of claim 1, comprising 25 to 500 μg bamosamine, 25 to 750 μg echinaxanthol, 100 to 3,000 μg bisdemethoxycurcumin, 50 to 500 μg daphniyunnine E, 500 to 75,000 μg curcumin, 50 to 500 μg decadienal/santolina epoxide, 10 to 500 μg eugenol, 0200 to 3,000 μg methoxycoumarin, 100 to 2,000 μg elijopyrone D, and 50 to 500 μg epierythrostominol per 100 mg of extract.

5. The turmeric extract of claim 1, comprising 25 to 500 μg bamosamine, 25 to 750 μg echinaxanthol, 100 to 3,000 μg bisdemethoxycurcumin, 50 to 500 μg daphniyunnine E, 500 to 75,000 μg curcumin, 50 to 1,000 μg lysine, 100 to 3,000 μg methoxycoumarin, 10 to 500 μg ethoxycoumarin, 10 to 500 μg α-phenylindol, 50 to 1,000 μg 3,4-dihydroscopoletin, 50 to 5,000 μg vasicinone, 50 to 5,000 μg 11-epileontidane, 10 to 500 μg methoxyflavanone, 50 to 500 μg aconitic acid triethyl ester, 50 to 500 μg 5,7-dimethoxyflavanone, 10 to 1,000 μg piperine, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

6. The turmeric extract of claim 1, comprising 25 to 500 μg bamosamine, 25 to 750 μg echinaxanthol, 100 to 3,000 μg bisdemethoxycurcumin, 50 to 500 μg daphniyunnine E, 500 to 75,000 μg curcumin, 50 to 500 μg decadienal/santolina epoxide, 10 to 500 μg eugenol, 0200 to 3,000 μg methoxycoumarin, 100 to 2,000 μg elijopyrone D, 50 to 500 μg epierythrostominol, 50 to 1,000 μg lysine, 10 to 500 μg ethoxycoumarin, 10 to 500 μg α-phenylindol, 50 to 1,000 μg 3,4-dihydroscopoletin, 50 to 5,000 μg vasicinone, 50 to 5,000 μg 11-epileontidane, 10 to 500 μg methoxyflavanone, 50 to 500 μg aconitic acid triethyl ester, 50 to 500 μg 5,7-dimethoxyflavanone, 10 to 1,000 μg piperine, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin.

7. The extract of claim 1, comprising 25 to 750 μg echinaxanthol, 100 to 3,000 μg bisdemethoxycurcumin, 500 to 75,000 μg curcumin per 100 mg of extract, 50 to 500 μg decadienal/santolina epoxide, 10 to 500 μg eugenol, and 100 to 5,000 μg vitamin H (biotin) per 100 mg of extract.

8. The extract of claim 1, comprising 25 to 750 μg echinaxanthol, 100 to 3,000 μg bisdemethoxycurcumin, 500 to 75,000 μg curcumin per 100 mg of extract, 100 to 1,000 μg ephemeranthone, 1000 to 10,000 μg demethoxycurcumin, 100 to 1,000 μg zopfinol, and 100 to 1,000 μg (+)-fargesin per 100 mg of extract.

9. The extract of claim 1, comprising 25 to 750 μg echinaxanthol, 100 to 3,000 μg bisdemethoxycurcumin, 500 to 75,000 μg curcumin per 100 mg of extract, 50 to 500 μg decadienal/santolina epoxide, 10 to 500 μg eugenol, 100 to 5,000 μg vitamin H (biotin), 100 to 1,000 μg ephemeranthone, 1000 to 10,000 μg demethoxycurcumin, 100 to 1,000 μg zopfinol, and 100 to 1,000 μg (+)-fargesin per 100 mg of extract.

10. The extract of claim 1, comprising 25 to 500 μg bamosamine, 100 to 3,000 μg bisdemethoxycurcumin, 500 to 75,000 μg curcumin, 10 to 500 μg eugenol, 0200 to 3,000 μg methoxycoumarin, 100 to 2,000 μg elijopyrone D, and 100 to 5,000 μg vitamin H (biotin) per 100 mg of extract.

11. The extract of claim 1, comprising 25 to 500 μg bamosamine, 100 to 3,000 μg bisdemethoxycurcumin, 500 to 75,000 μg curcumin, 50 to 1,000 μg lysine, 100 to 3,000 μg methoxycoumarin, 10 to 500 μg α-phenylindol, 50 to 1,000 μg 3,4-dihydroscopoletin, 50 to 5,000 μg 11-epileontidane, 10 to 500 μg methoxyflavanone, 50 to 500 μg aconitic acid triethyl ester, 50 to 500 μg 5,7-dimethoxyflavanone, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

12. The extract of claim 1, comprising 25 to 500 μg bamosamine, 100 to 3,000 μg bisdemethoxycurcumin, 500 to 75,000 μg curcumin, 10 to 500 μg eugenol, 0200 to 3,000 μg methoxycoumarin, 100 to 2,000 μg elijopyrone D, 100 to 5,000 μg vitamin H (biotin), 50 to 1,000 μg lysine, 100 to 3,000 μg methoxycoumarin, 10 to 500 μg α-phenylindol, 50 to 1,000 μg 3,4-dihydroscopoletin, 50 to 5,000 μg 11-epileontidane, 10 to 500 μg methoxyflavanone, 50 to 500 μg aconitic acid triethyl ester, 50 to 500 μg 5,7-dimethoxyflavanone, 100 to 1,000 μg ephemeranthone, 100 to 1,000 μg neohesperidose, 1000 to 10,000 μg demethoxycurcumin, 100 to 1,000 μg zopfinol, 10 to 500 μg dehydroagastanol, and 100 to 1,000 μg (+)-fargesin per 100 mg of extract.

13. The extract of claim 1, wherein the extract blocks β-amyloid aggregation in vitro.

14. The extract of claim 1, wherein the extract blocks β-amyloid secretion in vitro.

15. The extract of claim 1, wherein the extract prevents β-amyloid accumulation in vivo in the brain of a mammal.

16. The extract of claim 1, wherein the extract blocks Tau hyper-phosphorylation in vivo in the brain of a mammal.

17. The extract of claim 1, wherein the extract reduces the pro-inflammatory response in vivo in a mammal.

18. A pharmaceutical composition comprising the extract of claim 1 and a pharmaceutically acceptable carrier.

19. A method of treating or preventing a neurodegenerative disorder in a subject in need thereof comprising administering to the subject a therapeutically effecting amount of the turmeric extract of claim 1.

20. The method of claim 19, wherein the neurodegenerative disorder is Alzheimer's disease.

21. The method of claim 19, wherein the neurodegenerative disorder is dementia.

22. The method of claim 19, wherein the method prevents β-amyloid accumulation in the brain of the subject.

23. The method of claim 22, wherein plaque burden is reduced in the brain of the subject.

24. The method of claim 23, wherein the plaque burden is reduced in the hippocampus, the enthorhinal cortex, or both.

25. The method of claim 19, wherein Tau hyper-phosphorylation is blocked in the brain of the subject.

26. The method of claim 19, wherein a pro-inflammatory response is reduced in the subject.

27. The method of claim 26, wherein the levels of cytokines IL-2 and IL-4 are enhanced.

28. The method of claim 27, wherein the ratio of IL-4 to IL-2 is increased.

29. A method of preparing a turmeric extract comprising:
extracting turmeric with supercritical carbon dioxide in a supercritical extraction vessel, wherein the extraction vessel has a pressure from 300 to 800 bar and temperature of 50 to 100° C.

30. A method of preparing a turmeric extract comprising extracting turmeric with a mixture of water and ethanol.

31. A turmeric extract prepared by a process comprising:
extracting turmeric with supercritical carbon dioxide in a supercritical extraction vessel, wherein the extraction vessel has a pressure from 300 to 800 bar and temperature of 50 to 100° C.

32. A turmeric extract prepared by a process comprising:
extracting turmeric with a mixture of water and ethanol.
